(19) United States
(12) Patent Application Publication
(10) Pub. No.: US 2007/0021439 A1
(43) Pub. Date: Jan. 25, 2007

(54) Methods of reducing risk of infection from pathogens with soluble amide and ester pyrazinoylguanidine sodium channel blockers

(75) Inventor: Michael R. Johnson, Chapel Hill, NC (US)

Correspondence Address: HUTCHISON LAW GROUP PLLC, PO BOX 31686, RALEIGH, NC, 27612, US

(73) Assignee: Parion Sciences, Inc.

(21) Appl. No.: 11/188,673

(22) Filed: Jul. 25, 2005

(51) Int. Cl. A61K 31/4965; A61K 31/497

(52) U.S. Cl. 514/255.05; 514/255.06

(57) ABSTRACT

Prophylactic treatment methods are provided for protection of individuals and/or populations against infection from airborne pathogens. In particular, prophylactic treatment methods are provided comprising administering a sodium channel blocker or pharmaceutically acceptable salts thereof to one or more members of a population at risk of exposure to or already exposed to one or more airborne pathogens, either from natural sources or from intentional release of pathogens into the environment.

### BACKGROUND

1. Field of the Invention

The present invention relates to the use of sodium channel blockers for prophylactic, post-exposure prophylactic, preventive or therapeutic treatment against diseases or conditions caused by pathogens, particularly pathogens which may be used in bioterrorism.

2. Description of the Related Art

In recent years, a variety of research programs and biodefense measures have been put into place to deal with concerns about the use of biological agents in acts of terrorism. These measures are intended to address concerns regarding bioterrorism or the use of microorganisms or biological toxins to kill people, spread fear, and disrupt society. For example, the National Institute of Allergy and Infectious Diseases (NIAID) has developed a Strategic Plan for Biodefense Research which outlines plans for addressing research needs in the broad area of bioterrorism and emerging and reemerging infectious diseases. According to the plan, the deliberate exposure of the civilian population of the United States to Bacillus anthracis spores revealed a gap in the nation's overall preparedness against bioterrorism: Moreover, the report details that these attacks uncovered an unmet need for tests to rapidly diagnose, vaccines and immunotherapies to prevent, and drugs and biologics to cure disease caused by agents of bioterrorism.

Much of the focus of the various research efforts has been directed to studying the biology of the pathogens identified as potentially dangerous as bioterrorism agents, studying the host response against such agents, developing vaccines against infectious diseases, evaluating the therapeutics currently available and under investigation against such agents, and developing diagnostics to identify signs and symptoms of threatening agents. Such efforts are laudable but, given the large number of pathogens which have been identified as potentially available for bioterrorism, these efforts have not yet been able to provide satisfactory responses for all possible bioterrorism threats. Additionally, many of the pathogens identified as potentially dangerous as agents of bioterrorism do not provide adequate economic incentives for the development of therapeutic or preventive measures by industry. Moreover, even if preventive measures such as vaccines were available for each pathogen which may be used in bioterrorism, the cost of administering all such vaccines to the general population is prohibitive.

Until convenient and effective treatments are available against every bioterrorism threat, there exists a strong need for preventative, prophylactic or therapeutic treatments which can prevent or reduce the risk of infection from pathogenic agents.

### BRIEF SUMMARY

The present invention provides such methods of prophylactic treatment. In one aspect, a prophylactic treatment method is provided comprising administering a prophylactically effective amount of a sodium channel blocker or a pharmaceutically acceptable salt thereof to an individual in need of prophylactic treatment against infection from one or more airborne pathogens.

In another aspect, a prophylactic treatment method is provided for reducing the risk of infection from an airborne pathogen which can cause a disease in a human, said method comprising administering an effective amount of a sodium channel blocker or a pharmaceutically acceptable salt thereof to the lungs of the human who may be at risk of infection from the airborne pathogen but is asymptomatic for the disease, wherein the effective amount of a sodium channel blocker or a pharmaceutically acceptable salt is sufficient to reduce the risk of infection in the human.

In another aspect, a post-exposure prophylactic treatment or therapeutic treatment method is provided for treating infection from an airborne pathogen comprising administering an effective amount of a sodium channel blocker or a pharmaceutically acceptable salt thereof to the lungs of an individual in need of such treatment against infection from an airborne pathogen.

The sodium channel blockers which may be used in the methods exemplified include sodium channel blockers corresponding to compounds according to Formula I. Formula I is represented as a class of pyrazinoylguanidine compounds represented by formula (I): 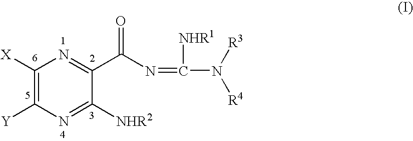
where X is hydrogen, halogen, trifluoromethyl, lower alkyl, unsubstituted or substituted phenyl, lower alkyl-thio, phenyl-lower alkyl-thio, lower alkyl-sulfonyl, or phenyl-lower alkyl-sulfonyl; Y is hydrogen, hydroxyl, mercapto, lower alkoxy, lower alkyl-thio, halogen, lower alkyl, unsubstituted or substituted mononuclear aryl, or —N(R²)₂; R¹ is hydrogen or lower alkyl; each R² is, independently, —R⁷, —(CH₂)ₘ—OR⁸, —(CH₂)ₘ—NR⁷R¹⁰, —(CH₂)ₙ(CHOR⁸)(CHOR⁸)ₙ—CH₂OR⁸, —(CH₂CH₂O)ₘ—R⁸, —(CH₂CH₂O)ₘ—CH₂CH₂NR⁷R¹⁰, —(CH₂)ₙ—C(═O)NR⁷R¹⁰, —(CH₂)ₙ-Z_{g}-R⁷, —(CH₂)ₘ—NR¹⁰—CH₂(CHOR⁸)(CHOR⁸)ₙ—CH₂OR⁸, —(CH₂)ₙ—CO₂R⁷, or 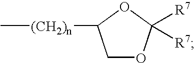
wherein when two —CH₂OR⁸ groups are located 1,2- or 1,3- with respect to each other the R⁸ groups may be joined to form a cyclic mono- or di-substituted 1,3-dioxane or 1,3-dioxolane;
R³ and R⁴ are each, independently, hydrogen, a group represented by formula (A), lower alkyl, hydroxy lower alkyl, phenyl, phenyl-lower alkyl, (halophenyl)-lower alkyl, lower-(alkylphenylalkyl), lower (alkoxyphenyl)-lower alkyl, naphthyl-lower alkyl, or pyridyl-lower alkyl, with the proviso that at least one of R³ and R⁴ is a group represented by formula (A): 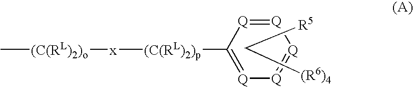
wherein

each R^{L} is, independently, —R⁷, —(CH₂)ₙ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose, 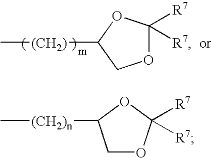
wherein when two —CH₂OR⁸ groups are located 1,2- or 1,3- with respect to each other the R⁸ groups may be joined to form a cyclic mono- or di-substituted 1,3-dioxane or 1,3-dioxolane;
   each o is, independently, an integer from 0 to 10;
   each p is an integer from 0 to 10;
   with the proviso that the sum of o and p in each contiguous chain is from 1 to 10;
   each x is, independently, O, NR¹⁰, C(═O), CHOH, C(═N—R¹⁰), CHNR⁷R¹⁰, or represents a single bond;
   each R⁵ is independently, —(CH₂)ₙ—CO₂R¹³, Het-(CH₂)ₘ—CO₂R¹³, —(CH₂)ₙ-Z_{g}-CO₂R¹³, Het-(CH₂)ₘ-Z_{g}-CO₂R¹³, —(CH₂)ₙ—NR¹⁰—(CH₂)ₘ(CHOR⁸)ₙ—CO₂R¹³, Het-(CH₂)ₘ—NR¹⁰—(CH₂)ₘ(CHOR⁸)ₙ—CO₂R¹³, —(CH₂)ₙ—(CHOR⁸)ₘ—CO₂R¹³, Het-(CH₂)ₘ—(CHOR⁸)ₘ—CO₂R¹³, —(CH₂)ₙ—(CHOR⁸)ₘ-Z_{g}-CO₂R¹³, Het-(CH₂)ₙ—(CHOR⁸)ₘ-Z_{g}-CO₂R¹³, —(CH₂)ₙ-Z_{g}-(CH₂)ₘ—CO₂R¹³, —(CH₂)ₙ-Z_{g}-(CH₂)ₘ—CO₂R¹³, —(CH₂)ₙ-Z_{g}(CHOR⁸)ₘ-Z_{g}-CO₂R¹³, Het-(CH₂)ₙ-Z_{g}-(CHOR⁸)ₘ-Z_{g}-CO₂R¹³, —(CH₂)ₙ—CONH—C(═NR¹³)—NR¹³R¹³, Het-(CH₂)ₙ—CO—NH—C(═NR¹³)—NR¹³R¹³, —(CH₂)ₙ-Z_{g}-CONH—C(═NR¹³)—NR¹³R¹³, Het-(CH₂)ₙ-Z_{g}-CONH—C(═NR¹³)—NR¹³R¹³, —(CH₂)ₙ—NR¹⁰—(CH₂)ₘ(CHOR⁸)ₙ—CONH—C(═NR¹³)—NR¹³R¹³, Het-(CH₂)ₙ—NR¹⁰—(CH₂)ₘ(CHOR⁸)ₙ—CONH—C(═NR¹³)—NR¹³R¹³, —(CH₂)ₙ—(CHOR⁸)ₘ—CONH—C(═NR¹³)—NR¹³R¹³, Het-(CH₂)ₙ—(CHOR⁸)ₘ—CONH—C(═NR¹³)—NR¹³R¹³, —(CH₂)ₙ—(CHOR⁸)ₘ-Z_{g}-CONH—C(═NR¹³)—NR¹³R¹³, Het-(CH₂)ₙ—(CHOR⁸)ₘ-Z_{g}-CONH—C(═NR¹³)—NR¹³R¹³, —(CH₂)ₙ-Z_{g}-(CH₂)ₘ—CONH—C(═NR¹³)—NR¹³R¹³, Het-(CH₂)ₙ-Z_{g}-(CH₂)ₘCONH—C(═NR¹³)—NR¹³R¹³, —(CH₂)ₙ-Z_{g}-(CHOR⁸)ₘ-Z_{g}-CONH—C(═NR¹³)—NR¹³R¹³, Het-(CH₂)ₙ-Z_{g}-(CHOR⁸)ₘ-Z_{g}-CONH—C(═NR¹³)—NR¹³R¹³, —(CH₂)ₙ—CONR⁷—CONR¹³R¹³, Het-(CH₂)ₙ—CONR⁷—CONR¹³R¹³, —(CH₂)ₙ-Z_{g}-CONR⁷—CONR¹³R¹³, —(CH₂)ₙ-Z_{g}-CONR⁷—CONR¹³R¹³, —(CH₂)ₙ—NR¹⁰—(CH₂)ₘ(CHOR⁸)ₙ—CONR⁷—CONR¹³R¹³, Het-(CH₂)ₙ—NR¹⁰—(CH₂)ₘ(CHOR⁸)ₙ—CONR⁷—CONR¹³R¹³, —(CH₂)ₙ—(CHOR⁸)ₘ—CONR⁷—CONR¹³R¹³, Het-(CH₂)ₙ—(CHOR⁸)ₘ—CONR⁷—CONR¹³R¹³, —(CH₂)ₙ—(CHOR⁸)ₘ-Z_{g}-CONR⁷—CONR¹³R¹³, Het-(CH₂)ₙ—(CHOR⁸)ₘ-Z_{g}-CNR⁷—CONR¹³R¹³, —(CH₂)ₙ-Z_{g}-(CH₂)ₘCONR⁷—CONR¹³R¹³, Het-(CH₂)ₙ-Z_{g}-(CH₂)ₘCONR⁷—CONR¹³R¹³, —(CH₂)ₙ-Z_{g}(CHOR⁸)ₘ-Z_{g}-CONR⁷—CONR¹³R¹³, Het-(CH₂)ₙ-Z_{g}(CHOR⁸)ₘ-Z_{g}-CONR⁷—CONR¹³R¹³, —(CH₂)ₙ—CONR⁷SO₂NR¹³R¹³, Het-(CH₂)ₘ—CONR⁷SO₂NR¹³R¹³, —(CH₂)ₙ-Z_{g}-CONR⁷SO₂NR¹³R¹³, Het-(CH₂)ₘ-Z_{g}-CONR⁷SO₂NR¹³R¹³, —(CH₂)ₙ—NR¹⁰—(CH₂)ₘ(CHOR⁸)ₙ—CONR⁷SO₂NR¹³R¹³, Het-(CH₂)ₘ—NR¹⁰—(CH₂)ₘ(CHOR⁸)ₙ—CONR⁷SO₂NR¹³R¹³ (CH₂)ₙ—(CHOR⁸)ₘ—CONR⁷SO₂NR¹³R¹³, Het-(CH₂)ₘ—(CHOR⁸)ₘ—CONR⁷SO₂NR¹³R¹³, —(CH₂)ₙ—(CHOR⁸)ₘ-Z_{g}-CONR⁷SO₂NR¹³R¹³, Het-(CH₂)ₙ—(CHOR⁸)ₘ-Z_{g}-CONR⁷SO₂NR¹³R¹³, —(CH₂)ₙ-Z_{g}-(CH₂)ₘCONR⁷SO₂NR¹³R¹³, Het-(CH₂)ₙ-Z_{g}-(CH₂)ₙ—CONR⁷SO₂NR¹³R¹³, —(CH₂)ₙ-Z_{g}-(CHOR⁸)ₘ-Z_{g}-CONR⁷SO₂NR¹³R¹³, Het-(CH₂)ₙ-Z_{g}-(CHOR⁸)ₘ-Z_{g}-CONR⁷SO₂NR¹³R¹³, —(CH₂)ₙ—SO₂NR¹³R¹³, Het-(CH₂)ₘ—SO₂NR¹³R¹³, —(CH₂)ₙ-Z_{g}-SO₂NR¹³R¹³, Het-(CH₂)ₘ-Z_{g}-SO₂NR¹³R¹³, —(CH₂)ₙ—NR¹⁰—(CH₂)ₘ(CHOR⁸)ₙ—SO₂NR¹³R¹³, Het-(CH₂)ₘ—NR¹³, —(CH₂)ₘ(CHOR⁸)ₙ—SO₂NR¹³R¹³, —(CH₂)ₙ—(CHOR⁸)ₘ—SO₂NR¹³R¹³, Het-(CH₂)ₘ—(CHOR⁸)ₘ—SO₂NR¹³R¹³, —(CH₂)ₙ—(CHOR⁸)ₘ-Z_{g}-SO₂NR¹³R¹³, Het-(CH₂)ₙ—(CHOR⁸)ₘ-Z_{g}-SO₂NR¹³R¹³, —(CH₂)ₙ-Z_{g}-(CH₂)ₘ—SO₂NR¹³R¹³, Het-(CH₂)ₙ-Z_{g}-(CH₂)ₘ—SO₂NR¹³R¹³, —(CH₂)ₙ-Z_{g}-(CHOR⁸)ₘ-Z_{g}-SO₂NR¹³R¹³, Het-(CH₂)ₙ-Z_{g}-(CHOR⁸)ₘ-Z_{g}-SO₂NR¹³R¹³, —(CH₂)ₙ—CONR¹³R¹³, Het-(CH₂)ₘ—CONR¹³R¹³, —(CH₂)ₙ-Z_{g}-CONR¹³R¹³, Het-(CH₂)ₘ-Z_{g}-CONR¹³R¹³, —(CH₂)ₙ—NR¹⁰—(CH₂)ₘ(CHOR⁸)ₙ—CONR¹³R¹³, Het-(CH₂)ₘ—NR¹⁰—(CH₂)ₘ(CHOR⁸)ₙ—CONR¹³R¹³, —(CH₂)ₙ—(CHOR⁸)ₘ—CONR¹³R¹³, Het-(CH₂)ₘ—(CHOR⁸)ₘ—CONR¹³R¹³, —(CH₂)ₙ—(CHOR⁸)ₘ-Z_{g}-CONR¹³R¹³, Het-(CH₂)ₙ—(CHOR⁸)ₘ-Z_{g}-CONR¹³R¹³, —(CH₂)ₙ-Z_{g}-(CH₂)ₘCONR¹³R¹³, Het-(CH₂)ₙ-Z_{g}-(CH₂)ₘCONR¹³R¹³, —(CH₂)ₙ-Z_{g}-(CHOR⁸)ₘ-Z_{g}-CONR¹³R¹³, Het-(CH₂)ₙ-Z_{g}-(CHOR⁸)ₘ-Z_{g}-CONR¹³R¹³, —(CH₂)ₙ—CONR⁷COR¹³, Het-(CH₂)ₘ—CONR⁷COR¹³, —(CH₂)ₙ-Z_{g}-CONR⁷COR¹³, Het-(CH₂)ₘ-Z_{g}-CONR⁷COR¹³, —(CH₂)ₙ—NR¹⁰—(CH₂)ₘ(CHOR⁸)ₙ—CONR⁷COR¹³, Het-(CH₂)ₘ—NR¹⁰—(CH₂)ₘ(CHOR⁸)ₙ—CONR⁷COR¹³, —(CH₂)ₙ—(CHOR⁸)ₘ—CONR⁷COR¹³, Het-(CH₂)ₘ—(CHOR⁸)ₘ—CONR⁷COR¹³, —(CH₂)ₙ—(CHOR⁸)ₘ-Z_{g}-CONR⁷COR¹³, Het-(CH₂)ₙ—(CHOR⁸)ₘ-Z_{g}-CONR⁷COR¹³, —(CH₂)ₙ-Z_{g}-(CH₂)ₘCONR⁷COR¹³, —(CH₂)ₙ-Z_{g}-(CH₂)ₘCONR⁷COR¹³, Het-(CH₂)ₙ-Z_{g}-(CHOR⁸)ₘ-Z_{g}-CONR⁷COR¹³, —(CH₂)ₙ—CONR⁷CO₂R¹³, —(CH₂)ₙ-Z_{g}-CONR⁷CO₂R¹³, Het-(CH₂)ₘ-Z_{g}-CONR⁷CO₂R¹³, —(CH₂)ₙ—NR¹⁰—(CH₂)ₘ(CHOR⁸)ₙ—CONR⁷CO₂R¹³, Het-(CH₂)ₘ—NR¹⁰—(CH₂)ₘ(CHOR⁸)ₙ—CONR⁷CO₂R¹³, —(CH₂)ₙ—(CHOR⁸)ₘ—CONR⁷CO₂R¹³, Het-(CH₂)ₘ—(CHOR⁸)ₘ—CONR⁷CO₂R¹³, —(CH₂)ₙ—(CHOR⁸)ₘ-Z_{g}-CONR⁷CO₂R¹³, Het-(CH₂)ₙ—(CHOR⁸)ₘ-Z_{g}-CONR⁷CO₂R¹³, —(CH₂)ₙ-Z_{g}(CH₂)ₘCONR⁷CO₂R¹³, Het-(CH₂)ₙ-Z_{g}-(CH₂)ₘCONR⁷CO₂R¹³, —(CH₂)ₙ-Z_{g}-(CHOR⁸)ₘ-Z_{g}-CONR⁷CO₂R¹³, Het-(CH₂)ₙ-Z_{g}-(CHOR⁸)ₘ-Z_{g}-CONR⁷CO₂R¹³, —(CH₂)ₙ—NH—C(═NR¹³)—NR¹³R¹³, Het-(CH₂)ₘ—NH—C(═NR¹³)—NR¹³R¹³, —(CH₂)ₙ-Z_{g}-NH—C(═NR¹³)—NR¹³R¹³, Het-(CH₂)ₘ-Z_{g}-NH—C(═NR¹³)—NR¹³R¹³, —(CH₂)ₙ—NR¹⁰—(CH₂)ₘ(CHOR⁸)ₙ—NH—C(═NR¹³)—NR¹³R¹³, Het-(CH₂)ₘ—NR¹⁰—(CH₂)ₘ(CHOR⁸)ₙ—NH—C(═NR¹³)—NR¹³R¹³, —(CH₂)ₙ—(CHOR⁸)ₘ—NH—C(═NR¹³)—NR¹³R¹³, Het-(CH₂)ₘ—(CHOR⁸)ₘ—NH—C(═NR¹³)—NR¹³R¹³, —(CH₂)ₙ—(CHOR⁸)ₘ-Z_{g}-NH—C(═NR¹³)—NR¹³R¹³, Het-(CH₂)ₙ—(CHOR⁸)ₘ-Z_{g}-NH—C(═NR¹³)—NR¹³R¹³, —(CH₂)ₙ-Z_{g}-(CH₂)ₘNH—C(═NR¹³)—NR¹³R¹³, Het-(CH₂)ₙ-Z_{g}-(CH₂)ₘNH—C(═NR¹³)—NR¹³R¹³, —(CH₂)ₙ-Z_{g}-(CHOR⁸)ₘ-Z_{g}-NH—C(═NR¹³)—NR¹³R¹³, Het-(CH₂)ₙ-Z_{g}-(CHOR⁸)ₘ-Z_{g}-NH—C(═NR¹³)—NR¹³R¹³, —(CH₂)ₙ—C(═NR¹³)—NR¹³R¹³, Het-(CH₂)ₘ—C(═NH)—NR¹³R¹³, —(CH₂)ₙ-Z_{g}-C(═NH)—NR¹³R¹³, Het-(CH₂)ₘ-Z_{g}-C(═NH)—NR¹³R¹³, —(CH₂)ₙ—NR¹⁰—(CH₂)ₘ(CHOR⁸)ₙ—C(═NR¹³)—NR¹³R¹³, Het-(CH₂)ₘ—NR¹⁰—(CH₂)ₘ(CHOR⁸)ₙ—C(═NR¹³)—NR¹³R¹³, —(CH₂)ₙ—(CHOR⁸)ₘ—C(═NR¹³)—NR¹³R¹³, Het-(CH₂)ₘ—(CHOR⁸)ₘ—C(═NR¹³)—NR¹³R¹³, —(CH₂)ₙ—(CHOR⁸)ₘ-Z_{g}-C(═NR¹³)—NR¹³R¹³, Het-(CH₂)ₙ—(CHOR⁸)ₙ-Z_{g}-C(═NR¹³)—NR¹³R¹³, —(CH₂)ₙ-Z_{g}-(CH₂)ₘ—C(═NHC(═NR¹³)—NR¹³R¹³, Het-(CH₂)ₙ-Z_{g}-(CH₂)ₘ—C(═N R¹³)—NR¹³R¹³, —(CH₂)ₙ-Z_{g}-(CHOR⁸)ₘ-Z_{g}-C(═NR¹³)—NR¹³R¹³, Het-(CH₂)ₙ-Z_{g}-(CHOR⁸)ₘ-Z_{g}-C(═NR¹³)—NR¹³R¹³;

wherein when two —CH₂OR⁸ groups are located 1,2- or 1,3- with respect to each other the R⁸ groups may be joined to form a cyclic mono- or di-substituted 1,3-dioxane or 1,3-dioxolane;

each R⁶ is, independently, —R⁵, —R⁷, —OR⁸, —N(R⁷)₂, —(CH₂)ₘ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose,

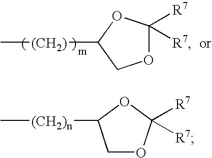

wherein when two R⁶ are —OR¹¹ and are located adjacent to each other on a phenyl ring, the alkyl moieties of the two R⁶ may be bonded together to form a methylenedioxy group, and

wherein when two —CH₂OR⁸ groups are located 1,2- or 1,3- with respect to each other the R⁸ groups may be joined to form a cyclic mono- or di-substituted 1,3-dioxane or 1,3-dioxolane;

each R⁷ is, independently, hydrogen, lower alkyl, phenyl, substituted phenyl or —CH₂(CHOR)⁸ₘ—R¹⁰;

each R⁸ is, independently, hydrogen, lower alkyl, —C(═O)—R¹¹, glucuronide, 2-tetrahydropyranyl, or

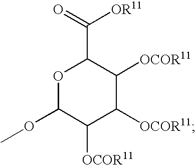

each R⁹ is, independently, —CO₂R⁷, —CON(R⁷)₂, —SO₂CH₃, or —C(═O)R⁷;

each R¹⁰ is, independently, —H, —SO₂CH₃, —CO₂R⁷—C(═O)NR⁷R⁹, —C(═O)R⁷, or —(CH₂)ₘ—(CHOH)ₙ—CH₂OH;

each Z is, independently, CHOH, C(═O), —(CH₂)ₙ—, CHNR⁷R¹⁰, C═NR¹⁰, or NR¹⁰;

each R¹¹ is, independently, lower alkyl;
each R¹² is independently, —SO₂CH₃, —CO₂R⁷, —C(═O)NR⁷R⁹, —C(═O)R⁷, or —CH₂—(CHOH), —CH₂OH;

each R¹³ is, independently, hydrogen, R⁷, R¹⁰, —(CH₂)ₘ—NR⁷R¹⁰, 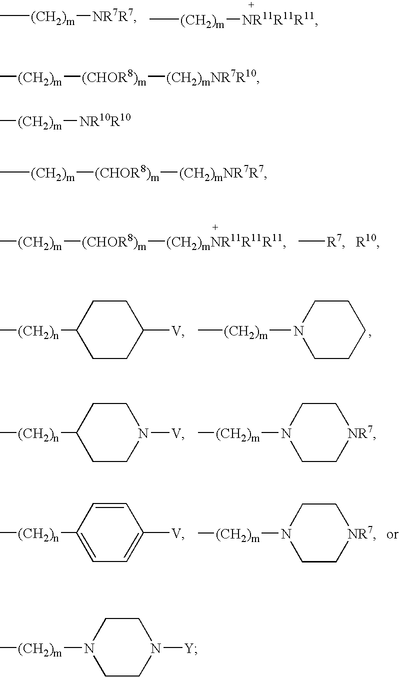
with the proviso that at least one R¹³ must be a group other than hydrogen, R⁷, or
R¹⁰;
   with the further proviso that NR¹³R¹³ can be joined on itself to form a ring comprising one of the following: 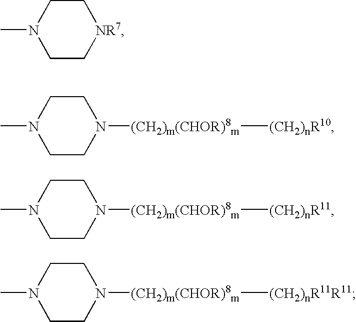
each Het is independently, —NR⁷, —NR¹⁰, —S—, —SO—, or —SO₂—; —O—, —SO₂NH—, —NHSO₂—, —NR⁷CO—, —CONR⁷—;
each g is, independently, an integer from 1 to 6;
each m is, independently, an integer from 1 to 7;
each n is, independently, an integer from 0 to 7;
each Q is, independently, C—R⁵, C—R⁶, or a nitrogen atom, wherein at
most three Q in a ring are nitrogen atoms;
each V is, independently, —(CH₂)ₘ—NR⁷R¹⁰, —(CH₂)ₘ—NR⁷R⁷, 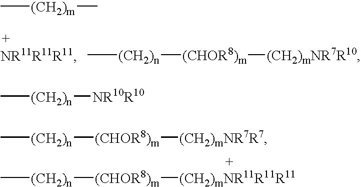
   with the proviso that when V is attached directly to a nitrogen atom, then V can also be, independently, R⁷, R¹⁰, or (R¹¹)₂;
wherein for any of the above compounds when two —CH₂OR⁸ groups are located 1,2- or 1,3- with respect to each other the R⁸ groups may be joined to form a cyclic mono- or di-substituted 1,3-dioxane or 1,3-dioxolane;
wherein any of the above compounds can be a pharmaceutically acceptable salt thereof, and wherein the above compounds are inclusive of all enantiomers, diastereomers, and racemic mixtures thereof.

In a preferred embodiment, each —(CH₂)ₙ-Z_{g}-C(═NH)_NR¹³R¹³ falls within the scope of the structures described above and is, independently,

—(CH₂)ₙ—CHNH₂(C═N)—N¹³R¹³.

In another preferred embodiment, each, Het-(CH₂)ₘ—NH—C(═NH)—NR¹³R¹³ falls within the scope of the structures described above and is, independently,

—(CH₂)ₙ—NH—C(═NH)NHR¹³.

In another preferred embodiment, each —(CH₂)ₙ-Z_{g}-(CHOR⁸)ₘ-Z_{g}-CONR¹³R¹³ falls within the scope of the structures described above and is, independently,

—(CH₂)ₙ—CONHCH₂(CHOH)ₘ—CONHR¹³.

In another preferred embodiment, each Het-(CH₂)ₙ-Z_{g}-(CHOR⁸)ₘ-Z_{g}-CONR¹³R¹³ falls within the scope of the structures described above and is, independently,

—NH—C(═O)—CH₂—(CHOH)ₙCH₂CONR¹³R¹³.

In another a preferred embodiment, each Het-(CH₂)ₘ-Z_{g}-C(═NH)—NR¹³R¹³ falls within the scope of the structures described above and is, independently,

—O—(CH₂)ₘ—NH—C(═NH)—N(R¹³)₂.

In another a preferred embodiment, each Het-(CH₂)ₘ-Z_{g}-CONR¹³R¹³ falls within the scope of the structures described above and is, independently,

—O—(CH₂)ₘ, —CHNH₂—CO₂NR¹³R¹³.

In another preferred embodiment, each R⁵ falls within the scope of the structures described above and is, independently,

—O—CH₂CHOHCH₂CONR¹³R¹³

—OCH₂CHOHCH₂CO₂R¹³ OCH₂CH₂CONR¹³R¹³

—OCH₂CH₂NHCOR¹³

—CH₂CH₂CONR¹³R¹³

—OCH₂CH₂CONR¹³R¹³O—(CH₂)ₘ—CO₂R¹³

—(CH₂)ₘ—CO₂R¹³

—OCH₂CH₂CO₂R¹³

—OCH₂CO₂R¹³

—O—(CH₂)ₘ—NH—C(═NH)—NR¹³)₂,

—(CH₂)ₙ—NH—C(═NH)—N(R¹)₂,

—NHCH₂(CHOH)₂CONR¹³R¹³

—OCH₂CO₂R¹³

—NHSO₂(CH₂)₂CONR¹³R¹³

—(CH₂)ₘ—NH—C(═O)—OR¹³

—O—(CH₂)ₘ—NH—C(═O)—OR¹³,

—(CH₂)ₙ—NH—C(═O)—R¹³,

—O—(CH₂)ₘ—NH—C(═O)—R¹³,

—O—CH₂C(═O)NR¹³R¹³

—CH₂NCO₂R¹³

—NHCO₂R¹³

—OCH₂CH₂CH₂CH₂CONR¹³R¹³

—SO₂CH₂CH₂CONR¹³R¹³

—OCH₂CH₂CHOHCH₂CONR¹³R¹³

—OCH₂CH₂NHCO₂R¹³

—NH—C(═NH₂)—NR¹³R¹³,

—OCH₂—(α-CHOH)₂—CONR¹³R¹³

—OCH₂CHOHCH₂CONHR¹³

—(CH₂)ₘ—CHOH—CH₂—NHCO₂R¹³

—O—(CH₂)ₘ—CHOH—CH₂—CO₂R¹³

—(CH₂)ₘ—NHC(O)OR¹³

—O—(CH₂)ₘ—NHC(O)OR¹³

—OCH₂CH₂CH₂CONHR¹³

—OCH₂CH₂NHCH₂(CHOH)₂CH₂CONHR¹³

—OCH₂CH₂CONH(CH₂[(CHOH)₂CH₂NH₂)]₂,

—(CH₂)₄—NHCO₂R¹³

—(CH₂)₄—CONR¹³R¹³,

—(CH₂)₄—CO₂R¹³

—OCH₂CH₂CONHSOCH₂CH₂N(CH₃)₂

—O—(CH₂)ₘ—C(═NH)N(R¹³)₂,

—(CH₂)ₘ—C(═NH)—N(R¹³)₂,

—(CH₂)₃—NHCO₂R¹³, —(CH₂)₃CONHCO₂R¹³

—O—(CH₂)ₘ—NH—NH—C(═NH)—N(R¹³)₂,

—(CH₂)ₙ—NH—NH—C(═NH)—N(R¹³)₂, or

—O—CH₂—CHOH—CH₂—NH—C(═NH)—N(R¹³)₂ or a pharmaceutically acceptable salt thereof, and inclusive of all enantiomers, diastereomers, and racemic mixtures thereof.

### DETAILED DESCRIPTION OF THE PREFERRED EMBODIMENTS

The prophylactic or therapeutic treatment methods of the present invention may be used in situations where a segment of the population has been, or is believed to have been, exposed to one or more airborne pathogens. The prophylactic or therapeutic treatment methods may additionally be used in situations of ongoing risk of exposure to or infection from airborne pathogens. Such situations may arise due to naturally occurring pathogens or may arise due to a bioterrorism event wherein a segment of the population is intentionally exposed to one or more pathogens. The individuals or portion of the population believed to be at risk from infection can be treated according to the methods disclosed herein. Such treatment preferably will commence at the earliest possible time, either prior to exposure if imminent exposure to a pathogen is anticipated or possible or after the actual or suspected exposure. Typically, the prophylactic treatment methods will be used on humans asymptomatic for the disease for which the human is believed to be at risk. The term “asymptomatic” as used herein means not exhibiting medically recognized symptoms of the disease, not yet suffering from infection or disease from exposure to the airborne pathogens, or not yet testing positive for a disease. The treatment methods may involve post-exposure prophylactic or therapeutic treatment, as needed.

Many of the pathogenic agents identified by NIAID have been or are capable of being aerosolized such that they may enter the body through the mouth or nose, moving into the bodily airways and lungs. These areas of the body have mucosal surfaces which naturally serve, in part, to defend against foreign agents entering the body. The mucosal surfaces at the interface between the environment and the body have evolved a number of “innate defense”, i.e., protective mechanisms. A principal form of such innate defense is to cleanse these surfaces with liquid. Typically, the quantity of the liquid layer on a mucosal surface reflects the balance between epithelial liquid secretion, often reflecting anion (Cl⁻ and/or HCO₃⁻) secretion coupled with water and a cation counter-ion, and epithelial liquid absorption, often reflecting Na⁺ absorption, coupled with water and counter anion (Cl⁻ and/or HCO₃⁻).

R. C. Boucher, in U.S. Pat. No. 6,264,975, describes methods of hydrating mucosal surfaces, particularly nasal airway surfaces, by administration of pyrazinoylguanidine sodium channel blockers. These compounds, typified by amiloride, benzamil and phenamil, are effective for hydration of the mucosal surfaces. U.S. Pat. No. 5,656,256, describes methods of hydrating mucous secretions in the lungs by administration of benzamil or phenamil, for example, to treat diseases such as cystic fibrosis and chronic bronchitis. U.S. Pat. No. 5,725,842 is directed to methods of removing retained mucus secretions from the lungs by administration of amiloride.

It has now been discovered that certain sodium channel blockers described and exemplified in U.S. patent application Ser. Nos. 10/920,391, filed Aug. 18, 2004, incorporated herein in its entirety by reference, may be used in prophylactic treatment methods to protect humans in whole or in part, against the risk of infection from pathogens which may or may not have been purposely introduced into the environment, typically into the air, of a populated area. Such treatment may be effectively used to protect those who may have been exposed where a vaccine is not available or has not been provided to the population exposed and/or in situations where treatments for the infection resulting from the pathogen to which a population has been subjected are insufficient or unavailable altogether.

Without being bound by any theory, it is believed that the sodium channel blockers disclosed herein surprisingly may be used on substantially normal or healthy lung tissue to prevent or reduce the uptake of airborne pathogens and/or to clear the lungs of all or at least a portion of such pathogens. Preferably, the sodium channel blockers will prevent or reduce the viral or bacterial uptake of airborne pathogens. The ability of sodium channel blockers to hydrate mucosal surfaces is believed to function to first hydrate lung mucous secretions, including mucous containing the airborne pathogens to which the human has been subjected, and then facilitate the removal of the lung mucous secretions from the body. By functioning to remove the lung mucous secretions from the body, the sodium channel blocker thus prevents or, at least, reduces the risk of infection from the pathogen(s) inhaled or brought into the body through a bodily airway.

The present invention is concerned primarily with the prophylactic, post exposure, rescue and therapeutic treatment of human subjects, but may also be employed for the treatment of other mammalian subjects, such as dogs and cats, for veterinary purposes, and to the extent the mammals are at risk of infection or disease from airborne pathogens.

The term “airway” as used herein refers to all airways in the respiratory system such as those accessible from the mouth or nose, including below the larynx and in the lungs, as well as air passages in the head, including the sinuses, in the region above the larynx.

The terms “pathogen” and “pathogenic agent” are interchangeable and, as used herein, mean any agent that can cause disease or a toxic substance produced by a pathogen that causes disease. Typically, the pathogenic agent will be a living organism that can cause disease. By way of example, a pathogen may be any microorganism such as bacterium, protozoan or virus that can cause disease.

The term “airborne pathogen” means any pathogen which is capable of being transmitted through the air and includes pathogens which travel through air by way of a carrier material and pathogens either artificially aerosolized or naturally occurring in the air.

The term “prophylactic” as used herein means the prevention of infection, the delay of infection, the inhibition of infection and/or the reduction of the risk of infection from pathogens and includes pre- and post-exposure to pathogens. The prophylactic effect may, inter alia, involve a reduction in the ability of pathogens to enter the body, or may involve the removal of all or a portion of pathogens which reach airways and airway surfaces in the body from the body prior to the pathogens initiating or causing infection or disease. The airways from which pathogens may be removed, in whole or part, include all bodily airways and airway surfaces with mucosal surfaces, including airway surfaces in the lungs.

The term “therapeutic” as used herein means to alleviate disease or infection from pathogens.

The compounds useful in this invention include sodium channel blockers such as those represented by Formula I. The sodium channel blockers may be prepared by the procedures described in U.S. patent application Ser. No. 10/920,391, filed Aug. 18, 2004, incorporated by reference herein in its entirety, in combination with procedures known to those skilled in the art.

Formula I may be represented as shown above. In the compounds represented by formula (I), X may be hydrogen, halogen, trifluoromethyl, lower alkyl, lower cycloalkyl, unsubstituted or substituted phenyl, lower alkyl-thio, phenyl-lower alkyl-thio, lower alkyl-sulfonyl, or phenyl-lower alkyl-sulfonyl. Halogen is preferred.

Examples of halogen include fluorine, chlorine, bromine, and iodine. Chlorine and bromine are the preferred halogens. Chlorine is particularly preferred. This description is applicable to the term “halogen” as used throughout the present disclosure.

As used herein, the term “lower alkyl” means an alkyl group having less than 8 carbon atoms. This range includes all specific values of carbon atoms and subranges there between, such as 1, 2, 3, 4, 5, 6, and 7 carbon atoms. The term “alkyl” embraces all types of such groups, e.g., linear, branched, and cyclic alkyl groups. This description is applicable to the term “lower alkyl” as used throughout the present disclosure. Examples of suitable lower alkyl groups include methyl, ethyl, propyl, cyclopropyl, butyl, isobutyl, etc.

Substituents for the phenyl group include halogens. Particularly preferred halogen substituents are chlorine and bromine.

Y may be hydrogen, hydroxyl, mercapto, lower alkoxy, lower alkyl-thio, halogen, lower alkyl, lower cycloalkyl, mononuclear aryl, or —N(R²)₂. The alkyl moiety of the lower alkoxy groups is the same as described above. Examples of mononuclear aryl include phenyl groups. The phenyl group may be unsubstituted or substituted as described above. The preferred identity of Y is —N(R²)₂. Particularly preferred are such compounds where each R² is hydrogen.

R¹ may be hydrogen or lower alkyl. Hydrogen is preferred for R¹.

Each R² may be, independently, —R⁷, —(CH₂)ₘ—OR⁸, —(CH₂)ₘ—NR⁷R¹⁰, —(CH₂)ₙ(CHOR⁸)(CHOR⁸)ₙ—CH₂OR⁸, —(CH₂CH₂O)ₘ—R⁸, —(CH₂CH₂O)ₘ—CH₂CH₂NR⁷R¹⁰, —(CH₂)ₙ—C(O)NR⁷R¹⁰, —(CH₂)ₙ-Z_{g}-R⁷, —(CH₂)ₘ—NR¹⁰—CH₂(CHOR⁸)(CHOR⁸)ₙ—CH₂OR⁸, —(CH₂)ₙ—CO₂R⁷, or

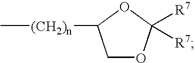

Hydrogen and lower alkyl, particularly C₁-C₃ alkyl are preferred for R². Hydrogen is particularly preferred.

R³ and R⁴ may be, independently, hydrogen, a group represented by formula (A), lower alkyl, hydroxy lower alkyl, phenyl, phenyl-lower alkyl, (halophenyl)-lower alkyl, lower-(alkylphenylalkyl), lower (alkoxyphenyl)-lower alkyl, naphthyl-lower alkyl, or pyridyl-lower alkyl, provided that at least one of R³ and R⁴ is a group represented by formula (A).

Preferred compounds are those where one of R³ and R⁴ is hydrogen and the other is represented by formula (A).

In formula (A), the moiety —(C(R^{L})₂)ₒ-x-(C(R^{L})₂)ₚ— defines an alkylene group bonded to the aromatic ring. The variables o and p may each be an integer from 0 to 10, subject to the proviso that the sum of o and p in the chain is from 1 to 10. Thus, o and p may each be 0, 1, 2, 3, 4, 5, 6, 7, 8, 9, or 10. Preferably, the sum of o and p is from 2 to 6. In a particularly preferred embodiment, the sum of o and p is 4.

The linking group in the alkylene chain, x, may be, independently, O, NR¹⁰, C(═O), CHOH, C(═N—R¹⁰), CHNR⁷R¹⁰, or represents a single bond.

Therefore, when x represents a single bond, the alkylene chain bonded to the ring is represented by the formula —(C(R^{L})₂)ₒ₊ₚ—, in which the sum o+p is from 1 to 10.

Each R^{L} may be, independently, —R⁷, —(CH₂)ₙ—OR⁸, —O—(CH₂)ₘ—OR⁸,

—(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸,

—O—(CH₂)ₘ(CHOR⁸)(CHOR⁸)ₙ—CH₂OR⁸, (CH₂CH₂O)ₘ—R⁸,

—O—(CH₂CH₂O)ₘ—R⁸, —(CH₂CH₂O)ₘ—CH₂CH₂NR⁷R¹⁰,

—O—(CH₂CH₂O)ₘ—CH₂CH₂NR⁷R¹⁰, —(CH₂)ₙ—C(═O)NR⁷R¹,

—O—(CH₂)ₘ—C(═O)NR⁷R¹⁰, —(CH₂)ₙ-(Z)_{g}-R⁷, —O—(CH₂)ₘ-(Z)_{g}-R⁷,

—(CH₂)ₙ—NR¹⁰—CH₂(CHOR⁸)(CHOR⁸), —CH₂OR⁸,

—O—(CH₂)ₘ—NR¹⁰—CH₂(CHOR⁸)(CHOR⁸)ₙ—CH₂OR⁸,

—(CH₂)ₙ—CO₂R⁷, —O—(CH₂)ₘ—CO₂R⁷—OSO₃H, —O-glucuronide, —O-glucose,

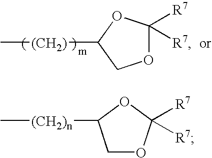

The preferred R^{L} groups include —H, —OH, —N(R⁷)₂, especially where each R⁷ is hydrogen.

In the alkylene chain in formula (A), it is preferred that when one R^{L} group bonded to a carbon atoms is other than hydrogen, then the other R^{L} bonded to that carbon atom is hydrogen, i.e., the formula —CHR^{L}—. It is also preferred that at most two R^{L} groups in an alkylene chain are other than hydrogen, where in the other R^{L} groups in the chain are hydrogens. Even more preferably, only one R^{L} group in an alkylene chain is other than hydrogen, where in the other R^{L} groups in the chain are hydrogens. In these embodiments, it is preferable that x represents a single bond.

In another particular embodiment of the invention, all of the R^{L} groups in the alkylene chain are hydrogen. In these embodiments, the alkylene chain is represented by the formula
—(CH₂)ₒ-x-(CH₂)ₚ—.

There is one R⁵ present on the ring in formula (A). Each R⁵ may be, independently,
—(CH₂)ₙ—CO₂R¹³, Het-(CH₂)ₘ—CO₂R¹³, —(CH₂)ₙ-Z_{g}-CO₂R¹³, Het-(CH₂)ₘ-Z_{g}-CO₂R¹³, —(CH₂)ₙ—NR¹⁰—(CH₂)ₘ(CHOR⁸)ₙ—CO₂R¹³, Het-(CH₂)ₘ—NR¹⁰—(CH₂)ₘ(CHOR⁸)ₙ—CO₂R¹³, —(CH₂)ₙ—(CHOR⁸)ₘ—CO₂R¹³, Het-(CH₂)ₘ—(CHOR⁸)ₘ—CO₂R¹³, —(CH₂)ₙ—(CHOR⁸)ₘ-Z_{g}-CO₂R¹³, Het-(CH₂)ₙ—(CHOR⁸)ₘ-Z_{g}-CO₂R¹³, —(CH₂)ₙ-Z_{g}-(CH₂)ₘ—CO₂R¹³, —(CH₂)ₙ-Z_{g}(CH₂)ₘ—CO₂R¹³, —(CH₂)ₙ-Z_{g}(CHOR⁸)ₘ-Z_{g}-CO₂R¹³, Het-(CH₂)ₙ-Z_{g}-(CHOR⁸)ₘ-Z_{g}-CO₂R¹³, —(CH₂)ₙ—CONH—C(═NR¹³)—NR¹³R¹³, Het-(CH₂)ₙ—CO—NH—C(═NR¹³)—NR¹³R¹³, —(CH₂)ₙ-Z_{g}-CONH—C(═NR¹³)—NR¹³R¹³, Het-(CH₂)ₙ-Z_{g}-CONH—C(═NR¹³)—NR¹³R¹³, —(CH₂)ₙ—NR¹⁰—(CH₂)ₘ(CHOR⁸)ₙ—CONH—C(═NR¹³)—NR¹³R¹³, Het-(CH₂)ₙ—NR¹⁰—(CH₂)ₘ(CHOR⁸)ₙ—CONH—C(═NR¹³)—NR¹³R¹³, —(CH₂)ₙ—(CHOR⁸)ₘ—CONH—C(═NR¹³)—NR¹³R¹³, Het-(CH₂)ₙ—(CHOR⁸)ₘ—CONH—C(═NR¹³)—NR¹³R¹³, —(CH₂)ₙ—(CHOR⁸)ₘ-Z_{g}-CONH—C(═NR¹³)—NR¹³R¹³, Het-(CH₂)ₙ—(CHOR⁸)ₘ-Z_{g}-CONH—C(═NR¹³)—NR¹³R¹³, —(CH₂)ₙ-Z_{g}-(CH₂)ₘCONH—C(═NR¹³)—NR¹³R¹³, Het-(CH₂)ₙ-Z_{g}-(CH₂)ₘCONH—C(═NR¹³)—NR¹³R¹³, —(CH₂)ₙ-Z_{g}-(CHOR⁸)ₘ-Z_{g}-CONH—C(═NR¹³)—NR¹³R¹³, Het-(CH₂)ₙ-Z_{g}-(CHOR⁸)ₘ-Z_{g}-CONH—C(═NR¹³)—NR¹³R¹³, —(CH₂)ₙ—CONR⁷—CONR¹³R¹³, Het-(CH₂)ₙ—CONR⁷—CONR¹³R¹³, —(CH₂)ₙ-Z_{g}-CONR⁷—CONR¹³R¹³, —(CH₂)ₙ-Z_{g}-CONR⁷—CONR¹³R¹³, —(CH₂)ₙ—NR¹⁰—(CH₂)ₘ(CHOR⁸)ₙ—CONR⁷—CONR¹³R¹³, Het-(CH₂)ₙ—NR¹⁰—(CH₂)ₘ(CHOR⁸)ₙ—CONR⁷—CONR¹³R¹³, —(CH₂)ₙ—(CHOR⁸)ₘ—CONR⁷—CONR¹³R¹³, Het-(CH₂)ₙ—(CHOR⁸)ₘ—CONR⁷—CONR¹³R¹³, —(CH₂)ₙ—(CHOR⁸)ₘ-Z_{g}-CONR⁷—CONR¹³R¹³, Het-(CH₂)ₙ—(CHOR⁸)ₘ-Z_{g}-CNR⁷—CONR¹³R¹³, —(CH₂)ₙ-Z_{g}-(CH₂)ₘCONR⁷—CONR¹³R¹³, Het-(CH₂)ₙ-Z_{g}-(CH₂)ₘCONR⁷—CONR¹³R¹³, —(CH₂)ₙ-Z_{g}(CHOR⁸)ₘ-Z_{g}-CONR⁷—CONR¹³R¹³—Het-(CH₂)ₙ-Z_{g}(CHOR⁸)ₘ-Z_{g}-CONR⁷—CONR¹³R¹³, —(CH₂)ₙ—CONR⁷SO₂NR¹³R¹³, Het-(CH₂)ₘ—CONR⁷SO₂NR¹³R¹³, —(CH₂)ₙ-Z_{g}-CONR⁷SO₂NR¹³R¹³, Het-(CH₂)ₘ-Z_{g}-CONR⁷SO₂NR¹³R¹³, —(CH₂)ₙ—NR¹⁰—(CH₂)ₘ(CHOR⁸)ₙ—CONR⁷SO₂NR¹³R¹³, Het-(CH₂)ₘ—NR¹⁰—(CH₂)ₘ(CHOR⁸)ₙ—CONR⁷SO₂NR¹³R¹³, —(CH₂)ₙ—(CHOR⁸)ₘ—CONR⁷SO₂NR¹³R¹³, Het-(CH₂)ₘ—(CHOR⁸)ₘ—CONR⁷SO₂NR¹³R¹³, —(CH₂)ₙ—(CHOR⁸)ₘ-Z_{g}-CONR⁷SO₂NR¹³R¹³, Het-(CH₂)ₙ—(CHOR⁸)ₘ-Z_{g}-CONR⁷SO₂NR¹³R¹³, —(CH₂)ₙ-Z_{g}-(CH₂)ₘCONR⁷SO₂NR¹³R¹³, Het-(CH₂)ₙ-Z_{g}-(CH₂)ₘCONR⁷SO₂NR¹³R¹³, —(CH₂)ₙ-Z_{g}-(CHOR⁸)ₘ-Z_{g}-CONR⁷SO₂NR¹³R¹³, Het-(CH₂)ₙ-Z_{g}-(CHOR⁸)ₘ-Z_{g}-CONR⁷SO₂NR¹³R¹³, —(CH₂)ₙ—SO₂NR¹³R¹³, Het-(CH₂)ₘ—SO₂NR¹³R¹³, —(CH₂)ₙ-Z_{g}-SO₂NR¹³R¹³, Het-(CH₂)ₘ-Z_{g}-SO₂NR¹³R¹³, —(CH₂)ₙ—NR¹⁰—(CH₂)ₘ(CHOR⁸)ₙ—SO₂NR¹³R¹³, Het-(CH₂)ₘ—NR¹⁰—(CH₂)ₘ(CHOR⁸)ₙ—SO₂NR¹³R¹³, —(CH₂)ₙ—(CHOR⁸)ₘ—SO₂NR¹³R¹³, Het-(CH₂)ₘ—(CHOR⁸)ₘ—SO₂NR¹³R¹³, —(CH₂)ₙ—(CHOR⁸)ₘ-Z_{g}-SO₂NR¹³R¹³, Het-(CH₂)ₙ—(CHOR⁸)ₘ-Z_{g}-SO₂NR¹³R¹³, —(CH₂)ₙ-Z_{g}-(CH₂)ₘSO₂NR¹³R¹³, Het-(CH₂)ₙ-Z_{g}-(CH₂)ₘSO₂NR¹³R¹³, —(CH₂)ₙ-Z_{g}-(CHOR⁸)ₘ-Z_{g}-SO₂NR¹³R¹³, Het-(CH₂)ₙ-Z_{g}-(CHOR⁸)ₘ-Z_{g}-SO₂NR¹³R¹³, —(CH₂)ₙ—CONR¹³R¹³, Het-(CH₂)ₘ—CONR¹³R¹³, —(CH₂)ₙ-Z_{g}-CONR¹³R¹³, Het-(CH₂)ₘ-Z_{g}-CONR¹³R¹³, —(CH₂)ₙ—NR¹⁰—(CH₂)ₘ(CHOR⁸)ₙ—CONR¹³R¹³, Het-(CH₂)ₘ—NR¹⁰—(CH₂)ₘ(CHOR⁸)ₙ—CONR¹³R¹³, —(CH₂)ₙ—(CHOR⁸)ₘ—CONR¹³R¹³, Het-(CH₂)ₘ—(CHOR⁸)ₘ—CONR¹³R¹³, —(CH₂)ₙ—(CHOR⁸)ₘ-Z_{g}-CONR¹³R¹³, Het-(CH₂)ₙ—(CHOR⁸)ₘ-Z_{g}-CONR¹³R¹³, —(CH₂)ₙ-Z_{g}-(CH₂)ₘCONR¹³R¹³, Het-(CH₂)ₙ-Z_{g}-(CH₂)ₘCONR¹³R¹³, —(CH₂)ₙ-Z_{g}-(CHOR⁸)ₘ-Z_{g}-CONR¹³R¹³, Het-(CH₂)ₙ-Z_{g}-(CHOR⁸)ₘ-Z_{g}-CONR¹³R¹³, —(CH₂)ₙ—CONR⁷COR¹³, Het-(CH₂)ₘ—CONR⁷COR¹³, —(CH₂)ₙ-Z_{g}-CONR⁷COR¹³, Het-(CH₂)ₘ-Z_{g}-CONR⁷COR¹³, —(CH₂)ₙ—NR¹⁰—(CH₂)ₘ(CHOR⁸)ₙ—CONR⁷COR¹³, Het-(CH₂)ₘ—NR¹⁰—(CH₂)ₘ(CHOR⁸)ₙ—CONR⁷COR¹³, —(CH₂)ₙ—(CHOR⁸)ₘ—CONR⁷COR¹³, Het-(CH₂)ₘ—(CHOR⁸)ₘ—CONR⁷COR¹³, —(CH₂)ₙ—(CHOR⁸)ₘ-Z_{g}-CONR⁷COR¹³ Het-(CH₂)ₙ—(CHOR⁸)ₘ-Z_{g}-CONR⁷COR¹³, —(CH₂)ₙ-Z_{g}-(CH₂)ₘCONR⁷COR¹³, —(CH₂)ₙ-Z_{g}-(CH₂)ₘCONR⁷COR¹³, Het-(CH₂)ₙ-Z_{g}-(CHOR⁸)ₘ-Z_{g}-CONR⁷COR¹³, —(CH₂)ₙ—CONR⁷CO₂R¹³, —(CH₂)ₙ-Z_{g}-CONR⁷CO₂R¹³, Het-(CH₂)ₘ-Z_{g}-CONR⁷COR¹³, —(CH₂)ₙ—NR¹⁰—(CH₂)ₘ(CHOR⁸)ₙ—CONR⁷CO₂R¹³, Het-(CH₂)ₘ—NR¹⁰—(CH₂)ₘ(CHOR⁸)ₙ—CONR⁷CO₂R¹³, —(CH₂)ₙ—(CHOR⁸)ₘ—CONR⁷CO₂R¹³, Het-(CH₂)ₘ—(CHOR⁸)ₘ—CONR⁷CO₂R¹³, —(CH₂)ₙ—(CHOR⁸)ₘ-Z_{g}-CONR⁷CO₂R¹³, Het-(CH₂)ₙ—(CHOR⁸)ₘ-Z_{g}-CONR⁷CO₂R¹³, —(CH₂)ₙ-Z_{g}-(CH₂)ₘCONR⁷CO₂R¹³, Het-(CH₂)ₙ-Z_{g}-(CH₂)ₘCONR⁷CO₂R¹³, —(CH₂)ₙ-Z_{g}-(CHOR⁸)ₘ-Z_{g}-CONR⁷CO₂R¹³, Het-(CH₂)ₙ-Z_{g}-(CHOR⁸)ₘ-Z_{g}-CONR⁷CO₂R¹³, —(CH₂)ₙ—NH—C(═NR¹³)—NR¹³R¹³, Het-(CH₂)ₘ—NH—C(═NR¹³)—NR¹³R¹³—(CH₂)ₙ-Z_{g}-NH—C(═NR¹³)—NR¹³R¹³, Het-(CH₂)ₘ-Z_{g}-NH—C(═NR¹³)—NR¹³R¹³, —(CH₂)ₙ—NR¹⁰—(CH₂)ₘ(CHOR⁸)ₙ—NH—C(═NR¹³)—NR¹³R¹³, Het-(CH₂)ₘ—NR¹⁰—(CH₂)ₘ(CHOR⁸)ₘ—NH—C(═NR¹³)—NR¹³R¹³, —(CH₂)ₙ—(CHOR⁸)ₘ—NH—C(═NR¹³)—NR¹³R¹³, Het-(CH₂)ₘ—(CHOR⁸)ₘ—NH—C(═NR¹³)—NR¹³R¹³, —(CH₂)ₙ—(CHOR⁸)ₘ-Z_{g}-NH_C(═NR¹³)—NR¹³R¹³, Het-(CH₂)ₙ—(CHOR⁸)ₘ-Z_{g}-NH—C(═NR¹³)—NR¹³R¹³, —(CH₂)ₙ-Z_{g}-(CH₂)ₘ—NH—C(═NR¹³)—NR¹³R¹³, Het-(CH₂)ₙ-Z_{g}-(CH₂)ₘ—NH—C(═NR¹³)—NR¹³R¹³, —(CH₂)ₙ-Z_{g}-(CHOR⁸)ₘ-Z_{g}-NH—C(═NR¹³)—NR¹³R¹³, Het-(CH₂)ₙ-Z_{g}-(CHOR⁸)ₘ-Z_{g}-NH—C(═NR¹³)—NR¹³R¹³, —(CH₂)ₙ—C(═NR¹³)—NR¹³R¹³, Het-(CH₂)ₘ—C(═NH)—NR¹³R¹³, —(CH₂)ₙ-Z_{g}-C(═NH)—NR¹³R¹³, Het-(CH₂)ₘ-Z_{g}-C(═NH)—NR¹³R¹³, —(CH₂)ₙ—NR¹⁰—(CH₂)ₘ(CHOR⁸)ₙ—C(═NR¹³)—NR¹³R¹³, Het-(CH₂)ₘ—NR¹⁰—(CH₂)ₘ(CHOR⁸), —C(═NR¹³)—NR¹³R¹³, —(CH₂)ₙ—(CHOR⁸)ₘ—C(═NR¹³)—NR¹³R¹³, Het-(CH₂)ₘ—(CHOR⁸)ₘ—C(═NR¹³)—NR¹³R¹³, —(CH₂)ₙ—(CHOR⁸)ₘ-Z_{g}-C(═NR¹³)—NR¹³R¹³, Het-(CH₂)ₙ—(CHOR⁸)ₘ-Z_{g}-C(═NR¹³)—NR¹³R¹³, —(CH₂)ₙ-Z_{g}-(CH₂)ₘ—C(═NHC(═NR¹³)—NR¹³R¹³, Het-(CH₂)ₙ-Z_{g}-(CH₂)ₘ—C(═NR¹³)—NR¹³R¹³, —(CH₂)ₙ-Z_{g}-(CHOR⁸)ₙ-Z_{g}-C(═NR¹³)—NR¹³R¹³, Het-(CH₂)ₙ-Z_{g}-(CHOR⁸)ₘ-Z_{g}-C(═NR¹³)—NR¹³R¹³;
   with the proviso wherein when two —CH₂OR⁸ groups are located 1,2- or 1,3- with respect to each other the R⁸ groups may be joined to form a cyclic mono- or di-substituted 1,3-dioxane or 1,3-dioxolane.

In a preferred embodiment, each —(CH₂)ₙ-Z_{g}-C(═NH)—NR¹³R¹³ falls within the scope of the structures described above and is, independently,

—(CH₂)ₙ—CHNH(C═N)—NR¹³R¹³.

In another preferred embodiment, each, Het-(CH₂)ₘ—NH—C(═NH)—NR¹³R¹³ falls within the scope of the structures described above and is, independently,

—(CH₂)ₙ—NH—C(═NH)NHR¹³

In another preferred embodiment, each —(CH₂)ₙ-Z_{g}-(CHOR⁸)ₘ-Z_{g}-CONR¹³R¹³ falls within the scope of the structures described above and is, independently,

—(CH₂)ₙ—CONHCH₂(CHOH)ₘ—CONHR¹³

In another preferred embodiment, each Het-(CH₂)ₙ-Z_{g}-(CHOR⁸)ₘ-Z_{g}-CONR¹³R¹³ falls within the scope of the structures described above and is, independently,

—NH—C(═O)—CH₂—(CHOH)ₙCH₂CONR¹³R¹³.

In another a preferred embodiment, each Het-(CH₂)ₘ-Z_{g}-C(═NH)—NR¹³R¹³ falls within the scope of the structures described above and is, independently,

—O—(CH₂)ₘ—NH—C(═NH)—N(R¹³)₂.

In another a preferred embodiment, each Het-(CH₂)ₘ-Z_{g}-CONR¹³R¹³ falls within the scope of the structures described above and is, independently,

—O—(CH₂)ₘ—CHNH₂—CO₂NR¹³R¹³.

In another preferred embodiment, each R⁵ falls within the scope of the structures described above and is, independently,

—O—CH₂CHOHCH₂CONR¹³R¹³

—OCH₂CHOHCH₂CO₂R¹³ OCH₂CH₂CONR¹³R¹³

—OCH₂CH₂NHCOR¹³

—CH₂CH₂CONR¹³R¹³

—OCH₂CH₂CONR¹³R¹³O—(CH₂)ₘ—CO₂R¹³

—(CH₂)ₘ—CO₂R¹³

—OCH₂CH₂CO₂R¹³

—OCH₂CO₂R¹³

—O—(CH₂)ₘ—NH—C(═NH)—NR¹³)₂,

—(CH₂)ₙ—NH—C(═NH)—N(R¹³)₂,

—NHCH₂(CHOH)₂—CONR¹³R¹³

—OCH₂CO₂R¹³

—NHSO₂(CH₂)₂CONR¹³R¹³

—(CH₂)ₘ—NH—C(═O)—OR¹³

—O—(CH₂)ₘ—NH—C(═O)—OR¹³,

—(CH₂)ₙ—NH—C(═O)—R¹³,

—O—(CH₂)ₘ—NH—C(═O)—R¹³,

—O—CH₂C(═O)NR¹³R¹³,

—CH₂NCO₂R¹³

—NHCO₂R¹³

—OCH₂CH₂CH₂CH₂CONR¹³R¹³

—SO₂CH₂CH₂CONR¹³R¹³

—OCH₂CH₂CHOHCH₂CONR¹³R¹³

—OCH₂CH₂NHCO₂R¹³

—NH—C(═NH₂)—NR¹³R¹³

—OCH₂-(α-CHOH)₂—CONR¹³R¹³

—OCH₂CHOHCH₂CONR¹³

—(CH₂)ₘ—CHOH—CH₂—NHCO₂R¹³

—O—(CH₂)ₘ—CHOH—CH₂—CO₂R¹³

—(CH₂)ₘ—NHC(O)OR¹³

—O—(CH₂)ₘ—NHC(O)OR¹³

—OCH₂CH₂CH₂CONHR¹³

—OCH₂CH₂NHCH₂(CHOH)₂CH₂CONHR¹³

—OCH₂CH₂CONH(CH₂[(CHOH)₂CH₂NH₂)]₂,

—(CH₂)₄—NHCO₂R¹³,

—(CH₂)₄—CONR¹³R¹³,

—(CH₂)₄—CO₂R¹³

—OCH₂CH₂CONHSOCH₂CH₂N(CH₃)₂

—O(CH₂)ₘ—C(═NH)N(R¹³)₂,

—(CH₂)ₘ—C(═NH)—N(R¹³)₂,

—(CH₂)₃—NHCO₂R¹³, —(CH₂)₃CONHCO₂R¹³

—O—(CH₂)ₘ—NH—NH—C(═NH)—N(R¹³)₂,

—(CH₂)ₙ—NH—NH—C(═NH)—N(R¹³)₂, or

—O—CH₂—CHOH—CH₂—NH—C(═NH)—N(R¹³)₂.

There are four R⁶ groups present on the ring in formula (A). Each R⁶ may be each, independently, —R⁷, —OR¹¹, —N(R⁷)₂, —(CH₂)ₘ—OR⁸,

—O—(CH₂)ₘ—OR⁸, —(CH₂)ₙ—NR⁷R¹⁰, —O—(CH₂)ₘ—NR⁷R¹⁰,

—(CH₂)ₙ(CHOR⁸)(CHOR⁸)ₙ—CH₂OR⁸, —O—(CH₂)ₘ(CHOR⁸)(CHOR⁸)ₙ—CH₂OR⁸,

—(CH₂CH₂O)ₘ—R⁸, —O—(CH₂CH₂O)ₘ—R⁸, —(CH₂CH₂O)ₘ—CH₂CH₂NR⁷R¹⁰,

—O—(CH₂CH₂O)ₘ—CH₂CH₂NR⁷R¹⁰, —(CH₂)ₙ—C(═O)NR⁷R¹⁰,

—O—(CH₂)ₘ—C(═O)NR⁷R¹⁰, —(CH₂)ₙ-(Z)_{g}-R⁷, —O—(CH₂)ₘ-(Z)_{g}-R⁷,

—(CH₂)ₙ—NR¹⁰—CH₂(CHOR⁸)(CHOR⁸)ₙ—CH₂OR⁸,

—O—(CH₂)ₘ—NR¹⁰—CH₂(CHOR⁸)(CHOR⁸)ₙ—CH₂OR⁸,

—(CH₂)ₙ—CO₂R⁷, —O—(CH₂)ₘ—CO₂R⁷, —OSO₃H, —O-glucuronide, —O-glucose, or

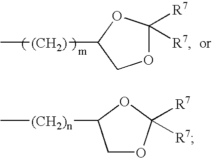

In addition, one of more of the R⁶ groups can be one of the R⁵ groups which fall within the broad definition of R⁶ set forth above.

When two R⁶ are —OR¹¹ and are located adjacent to each other on a phenyl ring, the alkyl moieties of the two R⁶ groups may be bonded together to form a methylenedioxy group, i.e., a group of the formula —O—CH₂—O—.

As discussed above, R⁶ may be hydrogen. Therefore, 1, 2, 3, or 4 R⁶ groups may be other than hydrogen. Preferably at most 3 of the R⁶ groups are other than hydrogen.

Each g is, independently, an integer from 1 to 6. Therefore, each g may be 1, 2, 3, 4, 5, or 6.

Each m is an integer from 1 to 7. Therefore, each m may be 1, 2, 3, 4, 5, 6, or 7.

Each n is an integer from 0 to 7. Therefore, each n maybe 0, 1, 2, 3, 4, 5, 6, or 7.

Each Q in formula (A) is C—R⁵, C—R⁶, or a nitrogen atom, where at most three Q in a ring are nitrogen atoms. Thus, there may be 1, 2, or 3 nitrogen atoms in a ring. Preferably, at most two Q are nitrogen atoms. More preferably, at most one Q is a nitrogen atom. In one particular embodiment, the nitrogen atom is at the 3-position of the ring. In another embodiment, each Q is either C—R⁵ or C—R⁶, i.e., there are no nitrogen atoms in the ring.

More specific examples of suitable groups represented by formula (A) are shown in formulas (B)-(E) below:

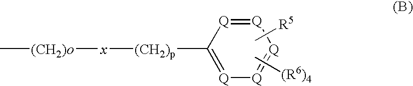

where o, x, p, R⁵, and R⁶, are as defined above;

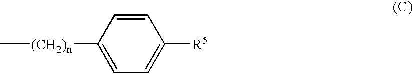

where n is an integer from 1 to 10 and R⁵ is as defined above;

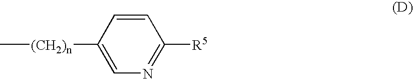

where n is an integer from 1 from 10 and R⁵ is as defined above;

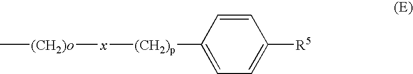

where o, x, p, and R⁵ are as defined above.

In a preferred embodiment, Y is —NH₂.

In another preferred embodiment, R² is hydrogen.

In another preferred embodiment, R¹ is hydrogen.

In another preferred embodiment, X is chlorine.

In another preferred embodiment, R³ is hydrogen.

In another preferred embodiment, R^{L} is hydrogen.

In another preferred embodiment, o is 4.

In another preferred embodiment, p is 0.

In another preferred embodiment, the sum of o and p is 4.

In another preferred embodiment, x represents a single bond.

In another preferred embodiment, R⁶ is hydrogen.

In another preferred embodiment, at most one Q is a nitrogen atom.

In another preferred embodiment, no Q is a nitrogen atom.

In a preferred embodiment:

X is halogen;

Y is —N(R⁷)₂;

R¹ is hydrogen or C₁-C₃ alkyl;

R² is R⁷—OR⁷, CH₂O⁷, or —CO₂R⁷;

R³ is a group represented by formula (A); and

R⁴ is hydrogen, a group represented by formula (A), or lower alkyl.

In another preferred embodiment:

X is chloro or bromo;

Y is —N(R⁷)₂;

R² is hydrogen or C₁-C₃ alkyl;

at most three R⁶ are other than hydrogen as described above;

at most three R^{L} are other than hydrogen as described above; and

at most 2 Q are nitrogen atoms.

In another preferred embodiment: Y is —NH₂.

In another preferred embodiment:

R⁴ is hydrogen;

at most one R^{L} is other than hydrogen as described above;

at most two R⁶ are other than hydrogen as described above; and

at most 1 Q is a nitrogen atom.

In another preferred embodiment, the compound of formula (I) is represented by the formula:

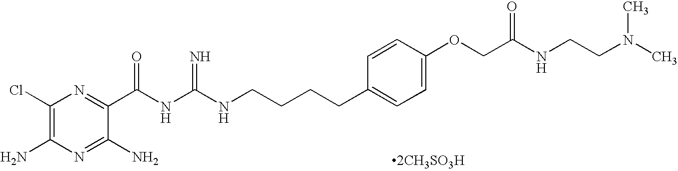

In another preferred embodiment, the compound of formula (I) is represented by the formula:

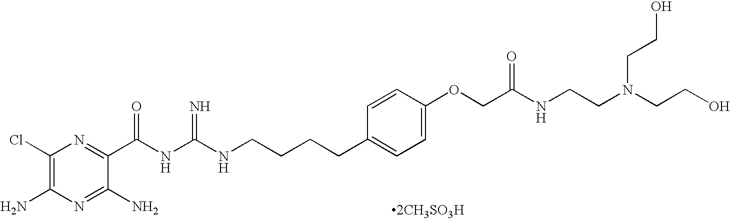

In another preferred embodiment, the compound of formula (I) is represented by the formula:

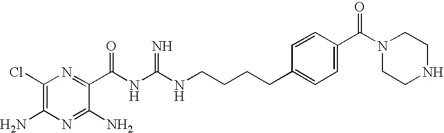

In another preferred embodiment, the compound of formula (D) is represented by the formula:

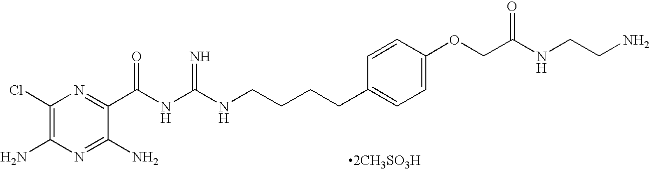

In another preferred embodiment, the compound of formula (I) is represented by the formula:

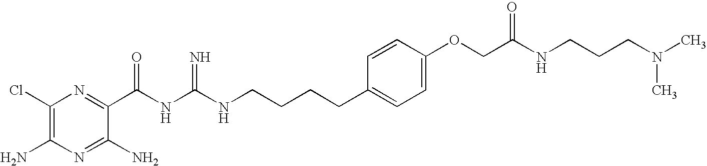

In another preferred embodiment, the compound of formula (I) is represented by the formula:

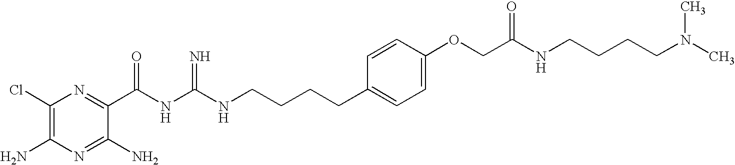

In another preferred embodiment, the compound of formula (I) is represented by the formula:

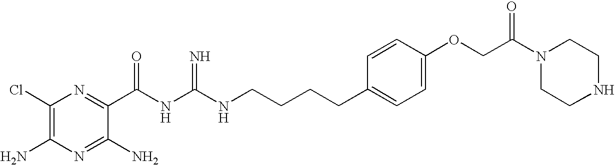

In another preferred embodiment, the compound of formula (I) is represented by the formula:

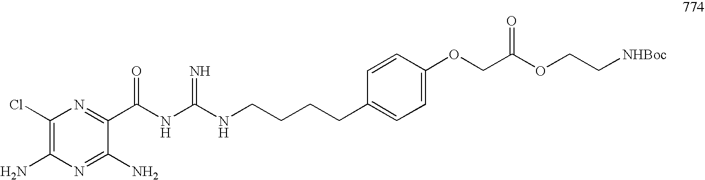

In another preferred embodiment, the compound of formula (I) is represented by the formula:

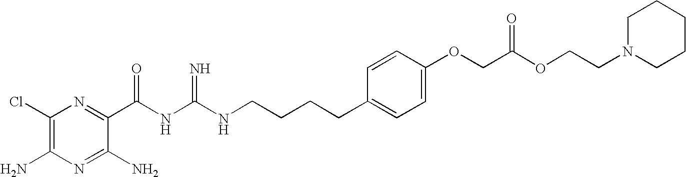

In another preferred embodiment, the compound of formula (I) is represented by the formula:

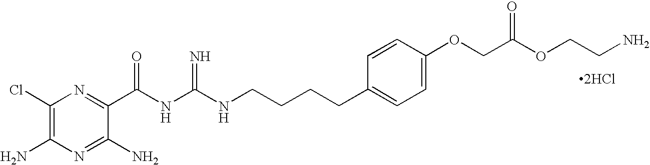

In another preferred embodiment, the compound of formula (I) is represented by the formula:

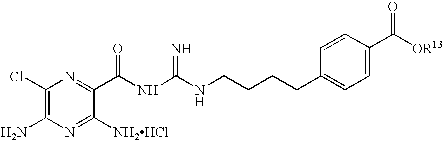

In another preferred embodiment, the compound of formula (I) is represented by the formula:

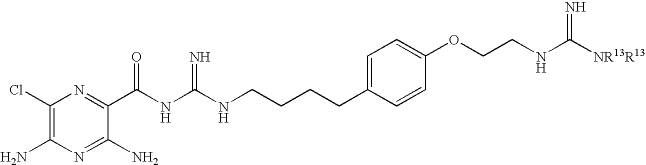

In another preferred embodiment, the compound of formula (I) is represented by the formula:

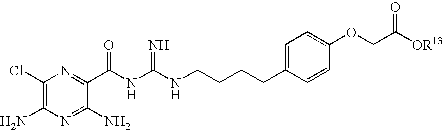

In another preferred embodiment, the compound of formula (I) is represented by the formula:

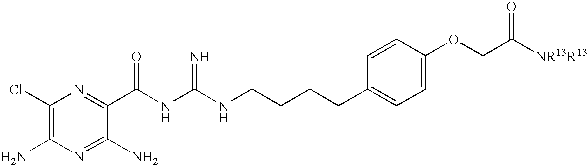

In another preferred embodiment, the compound of formula (I) is represented by the formula:

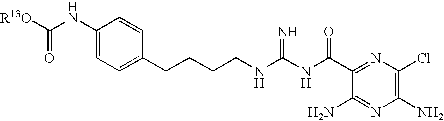

In another preferred embodiment, the compound of formula (I) is represented by the formula:

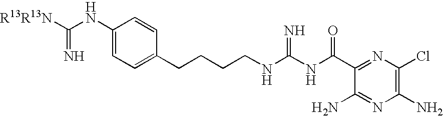

In another preferred embodiment, the compound of formula (I) is represented by the formula:

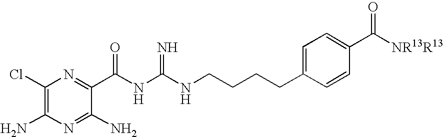

In another preferred embodiment, the compound of formula (I) is represented by the formula:

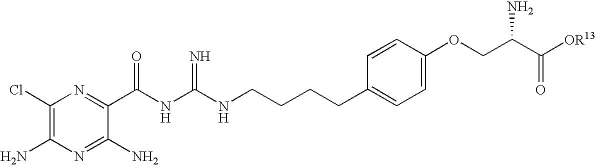

In another preferred embodiment, the compound of formula (I) is represented by the formula:

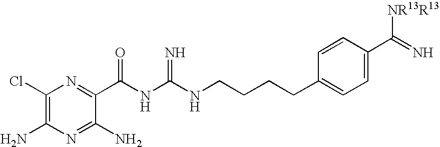

In another preferred embodiment, the compound of formula (I) is represented by the formula:

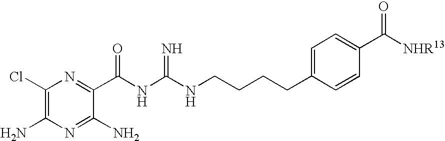

In another preferred embodiment, the compound of formula (I) is represented by the formula:

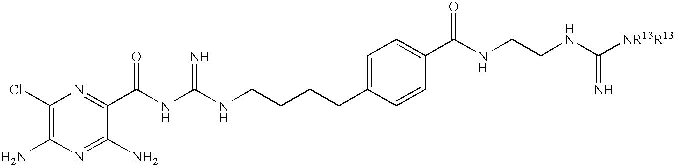

In another preferred embodiment, the compound of formula (I) is represented by the formula:

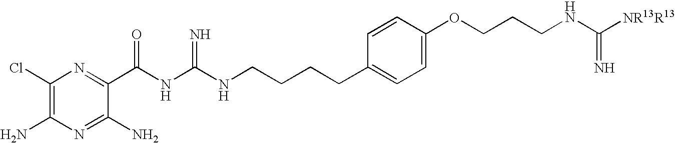

In another preferred embodiment, the compound of formula (I) is represented by the formula:

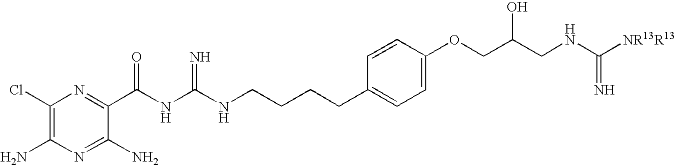

In another preferred embodiment, the compound of formula (I) is represented by the formula:

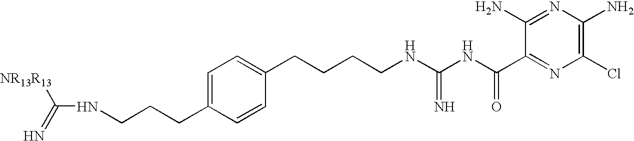

In another preferred embodiment, the compound of formula (I) is represented by the formula:

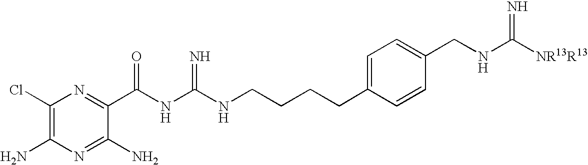

In another preferred embodiment, the compound of formula (I) is represented by the formula:

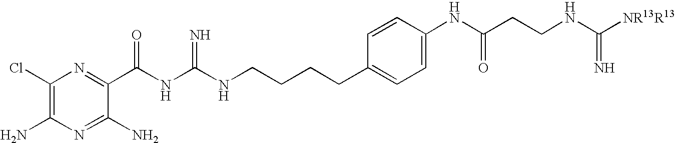

In another preferred embodiment, the compound of formula (I) is represented by the formula:

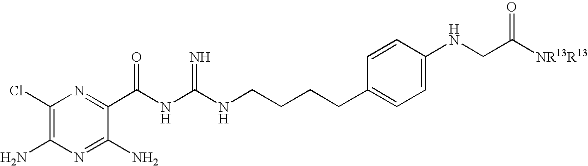

In another preferred embodiment, the compound of formula (I) is represented by the formula:

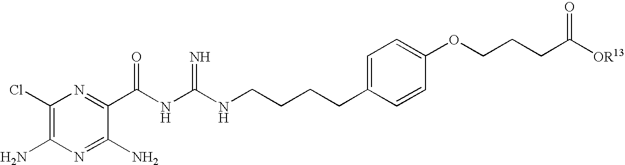

In another preferred embodiment, the compound of formula (I) is represented by the formula:

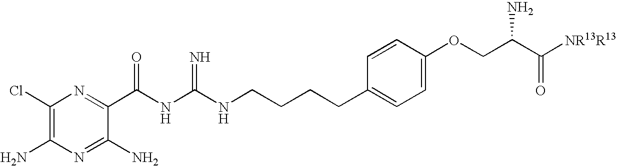

In another preferred embodiment, the compound of formula (I) is represented by the formula:

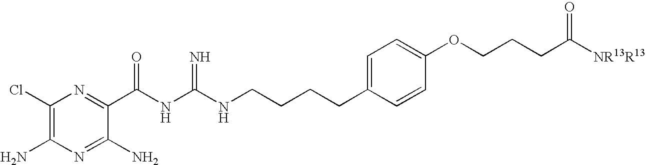

In another preferred embodiment, the compound of formula (I) is represented by the formula:

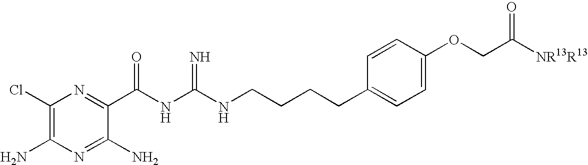

In another preferred embodiment, the compound of formula (I) is represented by the formula:

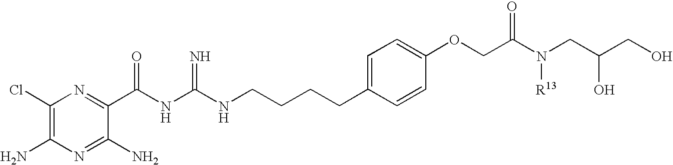

In another preferred embodiment, the compound of formula (I) is represented by the formula:

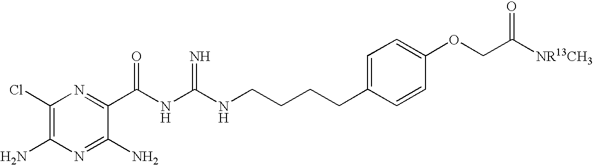

In another preferred embodiment, the compound of formula (I) is represented by the formula:

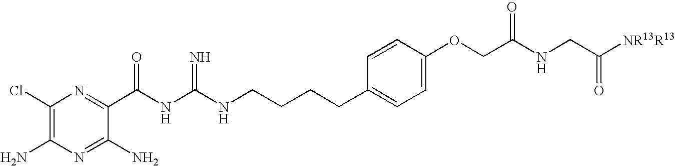

In another preferred embodiment, the compound of formula (I) is represented by the formula:

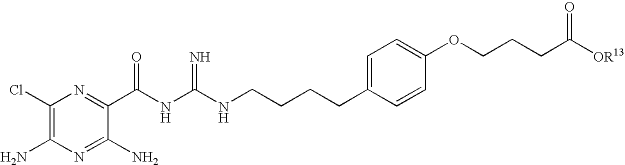

In another preferred embodiment, the compound of formula (I) is represented by the formula:

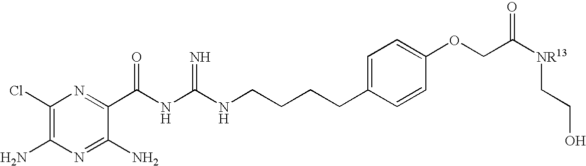

In another preferred embodiment, the compound of formula (I) is represented by the formula:

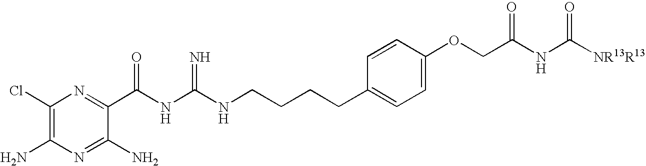

In another preferred embodiment, the compound of formula (I) is represented by the formula:

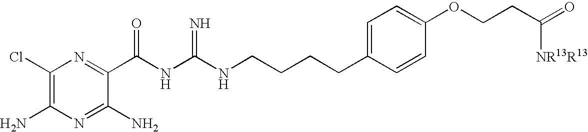

In another preferred embodiment, the compound of formula (D) is represented by the formula:

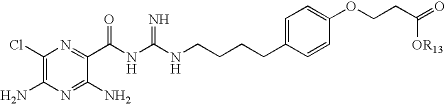

In another preferred embodiment, the compound of formula (I) is represented by the formula:

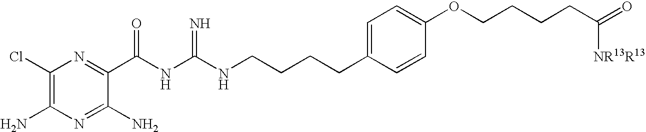

In another preferred embodiment, the compound of formula (I) is represented by the formula:

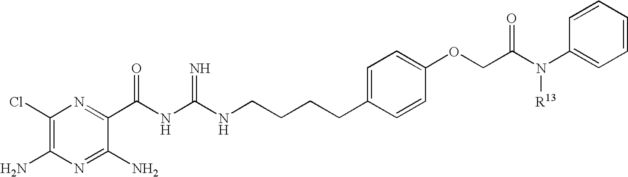

In another preferred embodiment, the compound of formula (I) is represented by the formula:

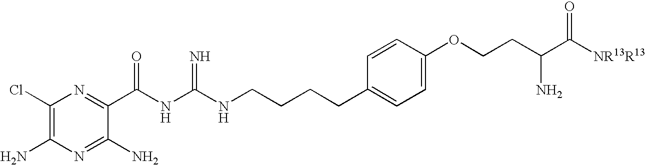

In another preferred embodiment, the compound of formula (I) is represented by the formula:

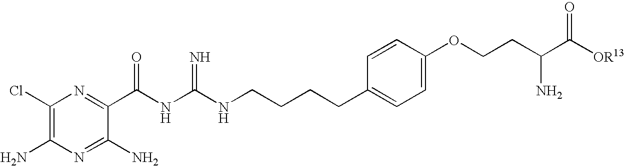

In another preferred embodiment, the compound of formula (I) is represented by the formula:

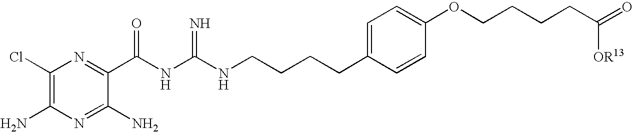

In another preferred embodiment, the compound of formula (I) is represented by the formula:

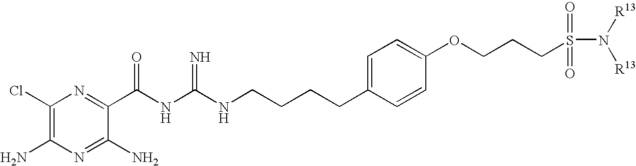

In another preferred embodiment, the compound of formula (I) is represented by the formula:

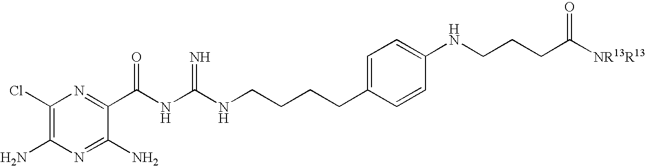

In another preferred embodiment, the compound of formula (I) is represented by the formula:

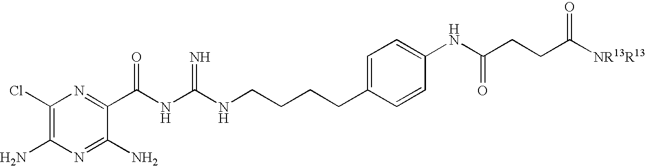

In another preferred embodiment, the compound of formula (I) is represented by the formula:

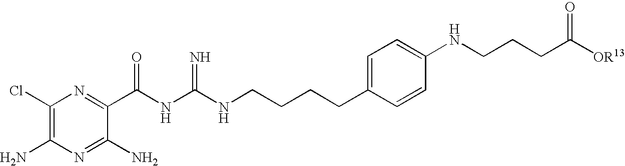

In another preferred embodiment, the compound of formula (D) is represented by the formula:

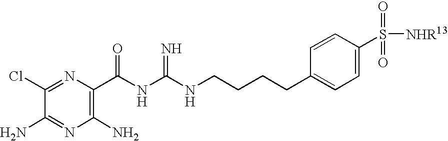

In another preferred embodiment, the compound of formula (I) is represented by the formula:

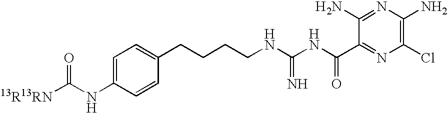

In another preferred embodiment, the compound of formula (I) is represented by the formula:

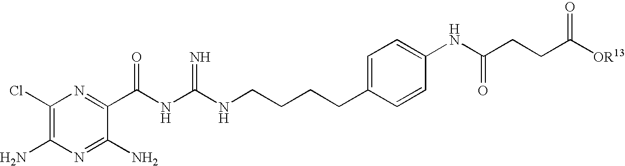

In another preferred embodiment, the compound of formula (I) is represented by the formula:

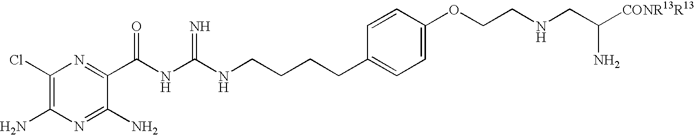

In another preferred embodiment, the compound of formula (I) is represented by the formula:

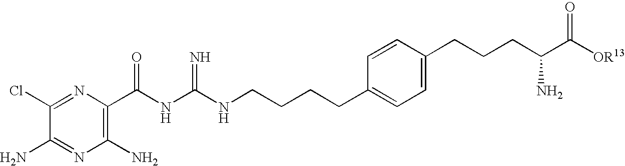

In another preferred embodiment, the compound of formula (I) is represented by the formula:

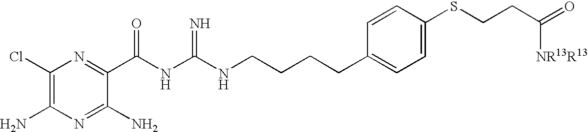

In another preferred embodiment, the compound of formula (I) is represented by the formula:

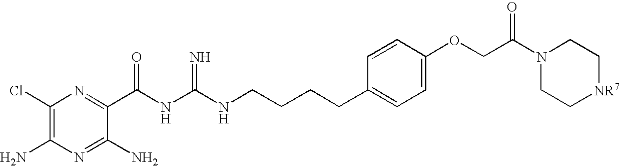

In another preferred embodiment, the compound of formula (I) is represented by the formula:

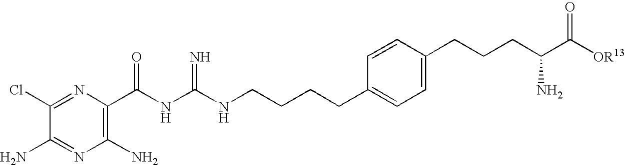

In another preferred embodiment, the compound of formula (I) is represented by the formula:

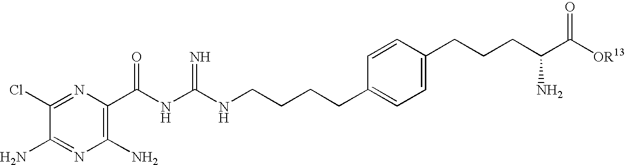

In another preferred embodiment, the compound of formula (I) is represented by the formula:

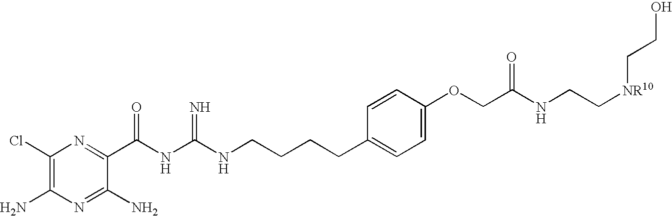

In another preferred embodiment, the compound of formula (I) is represented by the formula:

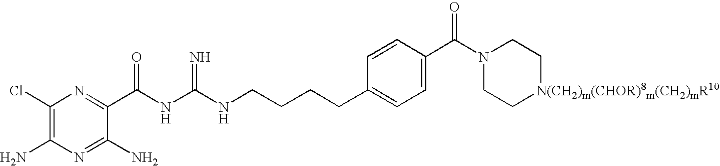

In another preferred embodiment, the compound of formula (I) is represented by the formula:

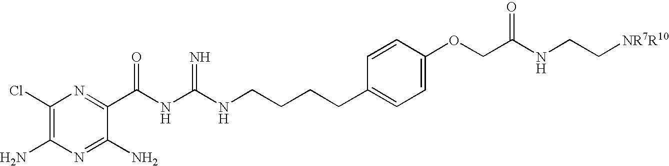

In another preferred embodiment, the compound of formula (I) is represented by the formula:

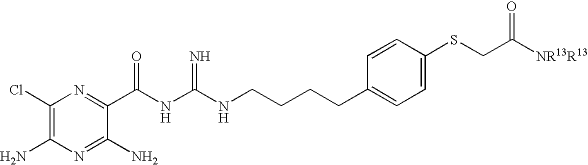

In another preferred embodiment, the compound of formula (I) is represented by the formula:

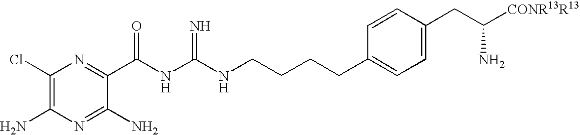

In another preferred embodiment, the compound of formula (I) is represented by the formula:

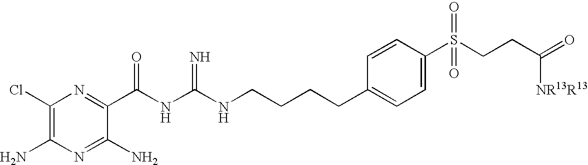

In another preferred embodiment, the compound of formula (I) is represented by the formula:

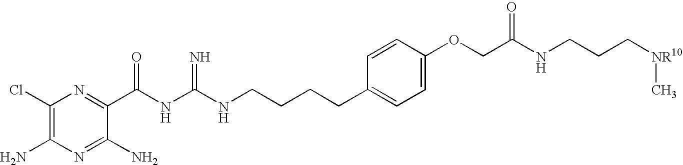

In another preferred embodiment, the compound of formula (D) is represented by the formula:

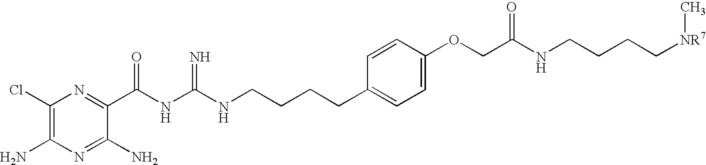

In another preferred embodiment, the compound of formula (I) is represented by the formula:

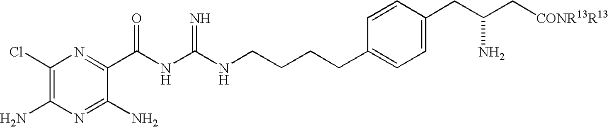

In another preferred embodiment, the compound of formula (I) is represented by the formula:

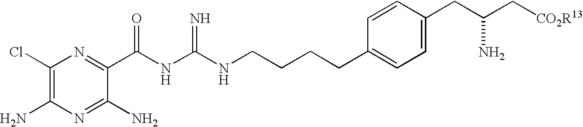

In another preferred embodiment, the compound of formula (I) is represented by the formula:

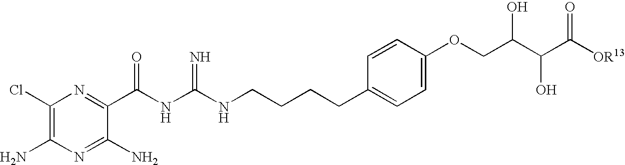

In another preferred embodiment, the compound of formula (I) is represented by the formula:

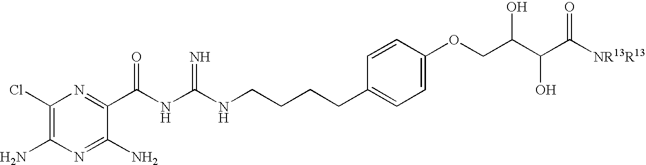

In another preferred embodiment, the compound of formula (I) is represented by the formula:

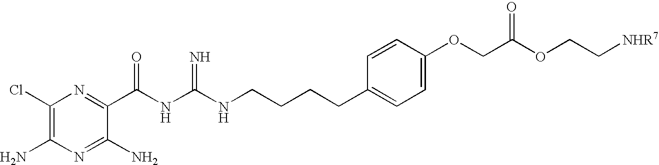

In another preferred embodiment, the compound of formula (I) is represented by the formula:

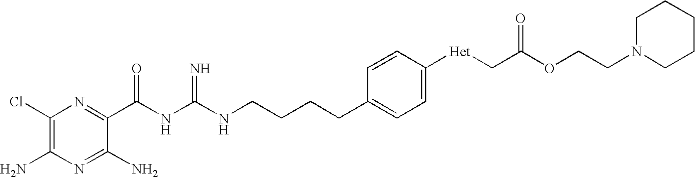

In another preferred embodiment, the compound of formula (I) is represented by the formula:

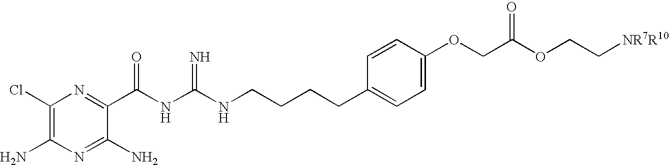

In another preferred embodiment, the compound of formula (I) is represented by the formula:

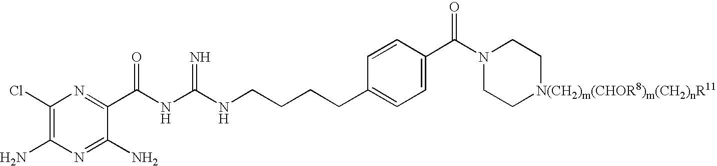

In another preferred embodiment, the compound of formula (D) is represented by the formula:

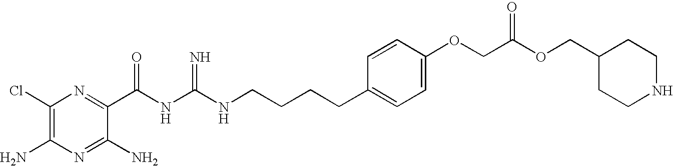

The compounds of formula (I) may be prepared and used as the free base. Alternatively, the compounds may be prepared and used as a pharmaceutically acceptable salt. Pharmaceutically acceptable salts are salts that retain or enhance the desired biological activity of the parent compound and do not impart undesired toxicological effects. Examples of such salts are (a) acid addition salts formed with inorganic acids, for example, hydrochloric acid, hydrobromic acid, sulfuric acid, phosphoric acid, nitric acid and the like; (b) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, malonic acid, sulfosalicylic acid, glycolic acid, 2-hydroxy-3-naphthoate, pamoate, salicylic acid, stearic acid, phthalic acid, mandelic acid, lactic acid and the like; and (c) salts formed from elemental anions for example, chlorine, bromine, and iodine.

It is to be noted that all enantiomers, diastereomers, and racemic mixtures of compounds within the scope of formula (I) are embraced by the present invention. All mixtures of such enantiomers and diastereomers are within the scope of the present invention.

The active compounds disclosed herein may be administered to the lungs of a patient by any suitable means but are preferably administered by administering an aerosol suspension of respirable particles comprised of the active compound, which the subject inhales. The compounds may be inhaled through the mouth or the nose. The active compound can be aerosolized in a variety of forms, such as, but not limited to, dry powder inhalants, metered dose inhalants or liquid/liquid suspensions. The quantity of sodium channel blocker included may be an amount sufficient to achieve the desired effect and as described in U.S. application Ser. No. 10/920,391, incorporated herein by reference.

Solid or liquid particulate sodium channel blocker prepared for practicing the present invention should include particles of respirable size: that is, particles of a size sufficiently small to pass through the mouth and larynx upon inhalation and into the bronchi and alveoli of the lungs. In general, particles ranging from about 1 to 5 microns in size (more particularly, less than about 4.7 microns in size) are respirable. Particles of non-respirable size which are included in the aerosol tend to be deposited in the throat and swallowed, and the quantity of non-respirable particles in the aerosol is preferably minimized. For nasal administration, a particle size in the range of 10-500 μm is preferred to ensure retention in the nasal cavity. Nasal administration may be useful where the pathogen typically enters through the nose. However, it is preferred to administer at least a portion of the sodium channel blocker in a dosage form which reaches the lungs to ensure effective prophylactic treatment in cases where the pathogen is expected to reach the lungs.

The dosage of active compound will vary depending on the prophylactic effect desired and the state of the subject, but generally may be an amount sufficient to achieve dissolved concentrations of active compound on the airway surfaces of the subject as described in the attached applications. Depending upon the solubility of the particular formulation of active compound administered, the daily dose may be divided among one or several unit dose administrations. The dosage may be provided as a prepackaged unit by any suitable means (e.g., encapsulating in a gelatin capsule).

Pharmaceutical formulations suitable for airway administration include formulations of solutions, emulsions, suspensions and extracts. See generally, J. Naim, Solutions, Emulsions, Suspensions and Extracts, in Remington: The Science and practice of Pharmacy, chap. 86 (19^{th} ed. 1995). Pharmaceutical formulations suitable for nasal administration may be prepared as described in U.S. Pat. No. 4,389,393 to Schor; U.S. Pat. No. 5,707,644 to Illum, U.S. Pat. No. 4,294,829 to Suzuki, and U.S. Pat. No. 4,835,142 to Suzuki.

In the manufacture of a formulation according to the invention, active agents or the physiologically acceptable salts or free bases thereof are typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a capsule, which may contain from 0.5% to 99% by weight of the active compound. One or more active compounds may be incorporated in the formulations of the invention, which formulations may be prepared by any of the well-known techniques of pharmacy consisting essentially of admixing the components.

Aerosols or mists of liquid particles comprising the active compound may be produced by any suitable means, such as, for nasal administration, by a simple nasal spray with the active compound in an aqueous pharmaceutically acceptable carrier such as sterile saline solution or sterile water. Other means include producing aerosols with a pressure-driven aerosol nebulizer or an ultrasonic nebulizer. See, e.g., U.S. Pat. No. 4,501,729. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation. Suitable formulations for use in nebulizers may consist of the active ingredient in a liquid carrier. The carrier is typically water (and most preferably sterile, pyrogen-free water) or a dilute aqueous alcoholic solution, preferably made isotonic with body fluids by the addition of, for example, sodium chloride.

Aerosols or mists of solid particles comprising the active compound may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. Such aerosol generators are known in the art. By way of example, see U.S. Pat. No. 5,725,842.

One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insulator consists either solely of the active ingredient or of a powder blend comprising the active ingredient, a suitable powder diluent, such as lactose, and an optional surfactant.

A second type of illustrative aerosol generator comprises a metered dose inhaler. Metered dose inhalers are pressurized aerosol dispensers, typically containing a suspension or solution formulation of the active ingredient in a liquefied propellant. During use these devices discharge the formulation through a valve adapted to deliver a metered volume, typically from 10 to 150 μl to produce a fine particle spray containing the active ingredient. Any propellant may be used in carrying out the present invention, including both chlorofluorocarbon-containing propellants and non-chlorofluorocarbon-containing propellants. Suitable propellants include certain chlorofluorocarbon compounds, for example, dichlorodifluoromethane, trichlorofluoromethane, dichlorotetrafluoroethane and mixtures thereof.

The formulation may additionally contain one or more co-solvents, for example, ethanol, surfactants, such as oleic acid or sorbitan trioleate, antioxidants, preservatives such as methyl hydroxybenzoate, volatile oils, buffering agents and suitable flavoring agents.

Compositions containing respirable dry particles of sodium channel blockers as described in the attached applications may be prepared as detailed in those applications. The active compound may be formulated alone (i.e., the solid particulate composition may consist essentially of the active compound) or in combination with a dispersant, diluent or carrier, such as sugars (i.e., lactose, sucrose, trehalose, mannitol) or other acceptable excipients for lung or airway delivery, which may be blended with the active compound in any suitable ratio (e.g., a 1 to 1 ratio by weight). The dry powder solid particulate compound may be obtained by methods known in the art, such as spray-drying, milling, freeze-drying, and the like.

The aerosol or mist, whether formed from solid or liquid particles, may be produced by the aerosol generator at a rate of from about 10 to about 150 liters per minute, more preferably from about 30 to about 150 liters per minute, and most preferably about 60 liters per minute. Aerosols containing greater amounts of medicament may be administered more rapidly.

Other medicaments may be administered with the active compounds disclosed if such medicament is compatible with the active compound and other ingredients in the formulation and can be administered as described herein.

The pathogens which may be protected against by the prophylactic post exposure, rescue and therapeutic treatment methods of the invention include any pathogens which may enter the body through the mouth, nose or nasal airways, thus proceeding into the lungs. Typically, the pathogens will be airborne pathogens, either naturally occurring or by aerosolization. The pathogens may be naturally occurring or may have been introduced into the environment intentionally after aerosolization or other method of introducing the pathogens into the environment. Many pathogens which are not naturally transmitted in the air have been or may be aerosolized for use in bioterrorism.

The pathogens for which the treatment of the invention may be useful includes, but is not limited to, category A, B and C priority pathogens as set forth by the NIAID. These categories correspond generally to the lists compiled by the Centers for Disease Control and Prevention (CDC). As set up by the CDC, Category A agents are those that can be easily disseminated or transmitted person-to-person, cause high mortality, with potential for major public health impact. Category B agents are next in priority and include those that are moderately easy to disseminate and cause moderate morbidity and low mortality. Category C consists of emerging pathogens that could be engineered for mass dissemination in the future because of their availability, ease of production and dissemination and potential for high morbidity and mortality.

Category A:
Bacillus anthracis (anthrax),
Clostridium botulinum (botulism),
Yersinia pestis (plague),
Variola major (smallpox) and other pox viruses,
Francisella tularensis (tularemia),
Viral hemorrhagic fevers
Arenaviruses,
   LCM (lymphocytic choriomeningitis), Junin virus,
Machupo virus, Guanarite virus,
   Lassa Fever,
Bunyaviruses,
   Hantavirus,
   Rift Valley Fever,
Flaviviruses,
   Dengue,
Filoviruses,
   Ebola
   Marburg;

Category B:
Burkholderia pseudomallei (melioidosis),
Coxiella burnetii (Q fever),
Brucella species (brucellosis),
Burkholderia mallei (glanders),
Ricin toxin from Ricinus communis,
Epsilon toxin of Clostridium perfringens,
Staphylococcal enterotoxin B,
Typhus fever (Rickettsia prowazekii),
Food and water-borne pathogens
   bacteria:
      Diarrheagenic Escherichia coli,
      Pathogenic vibrios,
      Shigella species,
      Salmonella species,
      Listeria monocytogenes,
      campylobacter jejuni,
      Yersinia enterocolitica;
   Viruses
      Caliciviruses,
      Hepatitis A;
   Protozoa
      Cryptosporidium parvum,
      Cyclospora cayatenensis,
      Giardia lamblia,
      Entamoeba histolytica,
      Toxoplasma,
      Microsporidia, and
   Additional viral encephalitides
      West Nile virus,
      LaCrosse,
      California encephalitis,
      Venezuelan equine encephalitis,
      Eastern equine encephalitis,
      Western equine encephalitis,
      Japanese encephalitis virus and
      Kyasanur forest virus, and

Category C: emerging infectious disease threats such as Nipah virus and additional hantaviruses, tickborne hemorrhagic fever viruses such as Crimean Congo hemorrhagic fever virus, tickborne encephalitis viruses, yellow fever, multi-drug resistant tuberculosis, influenza, other rickettsias and rabies.

Additional pathogens which may be protected against or the infection risk therefrom reduced include influenza viruses, rhinoviruses, adenoviruses and respiratory syncytial viruses, and the like. A further pathogen which may be protected against is the coronavirus which is believed to cause severe acute respiratory syndrome (SARS).

A number of the above-listed pathogens are known to be particularly harmful when introduced into the body through the air. For example, Bacillus anthracis, the agent which causes anthrax, has three major clinical forms, cutaneous, inhalational, and gastrointestinal. All three forms may lead to death but early antibiotic treatment of cutaneous and gastrointestinal anthrax usually cures those forms of anthrax. Inhalational anthrax, on the other hand, is a potentially fatal disease even with antibiotic treatment. Initial symptoms may resemble a common cold. After several days, the symptoms may progress to severe breathing problems and shock. For naturally occurring or accidental infections, even with appropriate antibiotics and all other available supportive care, the historical fatality rate is believed to be about 75 percent, according to the NIAID. Inhalational anthrax develops after spores are deposited in alveolar spaces and subsequently ingested by pulmonary alveolar macrophages. Surviving spores are then transported to the mediastinal lymph nodes, where they may germinate up to 60 days or longer. After germination, replicating bacteria release toxins that result in disease. This process is interrupted by administration of a prophylactically effective amount of a sodium channel blocker, as the spores may be wholly or partially eliminated from the body by removal of lung mucous secretions hydrated through the action of the sodium channel blocker.

Another pathogen of primary concern as one of the most dangerous potential biological weapons because it is easily transmitted from person to person, no effective therapy exists and few people carry full immunity to the virus, is the small pox virus, Variola major. Smallpox spreads directly from person to person, primarily by aerosolized saliva droplets expelled from an infected person. Initial symptoms include high fever, fatigue, headache and backache followed in two or three days by a characteristic rash.

The present invention provides a method of prophylactically treating one or more individuals exposed or potentially exposed to smallpox virus or other pox virus comprising the administration of a prophylactically effective amount of a sodium channel blocker. The administration of an effective amount of a sodium channel blocker will function to allow the Variola major virus or other pox virus present in the aerosolized saliva droplets to which the individual was exposed to be wholly or partially removed from the body by removal of hydrated lung mucous secretions hydrated through the action of the sodium channel blocker.

The bacterium Yersinia pestis causes plague and is widely available throughout the world. NIAID has reported that infection by inhalation of even small numbers of virulent aerosolized Y. pestis bacilli can lead to pneumonic plague, which has a mortality rate of almost 100% if left untreated. Pneumonic plague has initial symptoms of fever and cough which resemble other respiratory illnesses. Antibiotics are effective against plague but success with antibiotics depends on how quickly drug therapy is started, the dose of inhaled bacteria and the level of supportive care for the patient; an effective vaccine is not widely available.

The present invention provides a method of prophylactically treating one or more individuals exposed or potentially exposed to aerosolized Y. pestis bacilli comprising the administration of a sodium channel blocker. The administration of an effective amount of a sodium channel blocker will function to allow the aerosolized Y. pestis bacilli to be wholly or partially removed from the body by removal of hydrated lung mucous secretions hydrated through the action of the sodium channel blocker.

Botulinum toxin is another substance believed to present a major bioterrorism threat as it is easily released into the environment. Antibiotics are not effective against botulinum toxin and no approved vaccine exists. Although the toxin may be transmitted through food, the botulinum toxin is absorbed across mucosal surfaces and, thus, the present invention provides a method of prophylactically treating one or more individuals exposed or potentially exposed to botulinum toxin comprising the administration of a sodium channel blocker.

The NIAID has identified the bacteria that causes tularemia as a potential bioterrorist agent because Francisella tularensis is capable of causing infection with as few as ten organisms and due to its ability to be aerosolized. Natural infection occurs after inhalation of airborne particles. Tularemia may be treated with antibiotics and an experimental vaccine exists but knowledge of optimal therapeutic approaches for tularemia is limited because very few investigators are working on this disease. The present invention provides a method of prophylactically treating one or more individuals exposed or potentially exposed to aerosolized Francisella tularensis comprising the administration of a sodium channel blocker. The administration of an effective amount of a sodium channel blocker will function to allow the aerosolized Francisella tularensis to be wholly or partially removed from the body by removal of hydrated lung mucous secretions hydrated through the action of the sodium channel blocker.

The Category B and C bacteria most widely believed to have the potential to infect by the aerosol route include gram negative bacteria such as Brucella species, Burkholderia pseudomallei, Burkholderia mallei, Coxiella burnetii, and select Rickettsia spp. Each of these agents is believed to be capable of causing infections following inhalation of small numbers of organisms. Brucella spp. may cause brucellosis. Four of the six Brucella spp., B. suis, B. melitensis, B. abortus and B. canis, are known to cause brucellosis in humans. Burkholderia pseudomallei may cause melioidosis in humans and other mammals and birds. Burkholderia mallei, is the organism that causes glanders, normally a disease of horses, mules and donkeys but infection following aerosol exposure has been reported, according to NIAID. Coxiella burnetii, may cause Q fever and is highly infectious. Infections have been reported through aerosolized bacteria and inhalation of only a few organisms can cause infections. R. prowazekii, R. rickettsi, R. conorrni and R. typhi have been found to have low-dose infectivity via the aerosol route.

The present invention provides a method of prophylactically treating one or more individuals exposed or potentially exposed to aerosolized gram negative bacteria such as Brucella species, Burkholderia pseudomallei, Burkholderia mallei, Coxiella burnetii, and select Rickettsia spp comprising the administration of a sodium channel blocker. The administration of an effective amount of a sodium channel blocker will function to allow the aerosolized gram negative bacteria to be wholly or partially removed from the body by removal of hydrated lung mucous secretions hydrated through the action of the sodium channel blocker.

A number of typically arthropod-borne viruses are believed to pose a significant threat as potential bioterrorist weapons due to their extreme infectivity following aerosolized exposure. These viruses include arboviruses which are important agents of viral encephalitides and hemorrhagic fevers. Such viruses may include alphaviruses such as Venezuelan equine encephalitis virus, eastern equine encephalitis virus and western equine encephalitis virus. Other such viruses may include flaviviruses such as West Nile virus, Japanese encephalitis virus, Kyasanur forest disease virus, tick-borne encephalitis virus complex and yellow fever virus. An additional group of viruses which may pose a threat include bunyaviruses such as California encephalitis virus, or La Crosse virus, Crimean-Congo hemorrhagic fever virus. According to the NIAID, vaccines or effective specific therapeutics are available for only a very few of these viruses. In humans, arbovirus infection is usually initially asymptomatic or causes nonspecific flu-like symptoms such as fever, aches and fatigue.

The present invention provides a method of prophylactically treating one or more individuals exposed or potentially exposed to aerosolized arboviruses comprising the administration of a sodium channel blocker. The administration of an effective amount of a sodium channel blocker will function to allow the arboviruses to be wholly or partially removed from the body by removal of hydrated lung mucous secretions hydrated through the action of the sodium channel blocker.

Certain category B toxins such as ricin toxin from Ricinus communis, epsilon toxin of Clostridium perfringens and Staphylococcal enterotoxin B, also are viewed as potential bioterrorism tools. Each of these toxins may be delivered to the environment or population by inhalational exposure to aerosols. Low dose inhalation of ricin toxin may cause nose and throat congestion and bronchial asthma while higher dose inhalational exposure caused severe pneumonia, acute inflammation and diffuse necrosis of the airways in nonhuman primates. Clostridium perfringens is an anaerobic bacterium that can infect humans and animals. Five types of bacteria exist that produce four major lethal toxins and seven minor toxins, including alpha toxin, associated with gas gangrene, beta toxin, responsible for necrotizing enteritis, and epsilon toxin, a neurotoxin that leads to hemorrhagic enteritis in goats and sheep. Inhalation of Staphylococcus aureus has resulted in extremely high fever, difficulty breathing, chest pain and headache.

The present invention provides a method of prophylactically treating one or more individuals exposed or potentially exposed to aerosolized toxins comprising the administration of a sodium channel blocker. The administration of an effective amount of a sodium channel blocker will function to allow the aerosolized toxins to be wholly or partially removed from the body by removal of hydrated lung mucous secretions hydrated through the action of the sodium channel blocker.

Mycobacterium tuberculosis bacteria causes tuberculosis and is spread by airborne droplets expelled from the lungs when a person with tuberculosis coughs, sneezes or speaks. The present invention provides a method of prophylactically treating one or more individuals exposed or potentially exposed to Mycobacterium tuberculosis bacteria comprising the administration of a sodium channel blocker. The administration of an effective amount of a sodium channel blocker will function to allow the Mycobacterium tuberculosis bacteria to be wholly or partially removed from the body by removal of hydrated lung mucous secretions hydrated through the action of the sodium channel blocker.

The methods of the present invention may also be used against more common pathogens such as influenza viruses, rhinoviruses, adenoviruses and respiratory syncytial viruses (RSV). The present invention provides a method of prophylactically or therapeutically treating one or more individuals exposed or potentially exposed to one of these viruses comprising the administration of a sodium channel blocker. The administration of an effective amount of a sodium channel blocker will function to allow the virus to be wholly or partially removed from the body by removal of hydrated lung mucous secretions hydrated through the action of the sodium channel blocker.

The methods of the present invention may further be used against the virus believed to be responsible for SARS, the coronavirus. Severe acute respiratory syndrome is a respiratory illness that is believed to spread by person-to-person contact, including when someone coughs or sneezes droplets containing the virus onto others or nearby surfaces. The CDC currently believes that it is possible that SARS can be spread more broadly through the air or by other ways that are not currently known. Typically, SARS begins with a fever greater than 100.4° F. Other symptoms include headache and body aches. After two to seven days, SARS patients may develop a dry cough and have trouble breathing.

To the extent SARS is caused by an airborne pathogen, the present invention provides a method of prophylactically treating one or more individuals exposed or potentially exposed to the SARS virus comprising the administration of a sodium channel blocker. The administration of an effective amount of a sodium channel blocker will function to allow the virus to be wholly or partially removed from the body by removal of hydrated lung mucous secretions hydrated through the action of the sodium channel blocker.

The compounds of formula (D may be synthesized according to procedures known in the art. A representative synthetic procedure is shown in the scheme below: 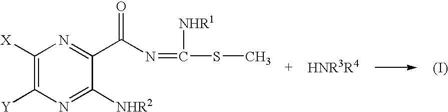
These procedures are described in, for example, E. J. Cragoe, “The Synthesis of Amiloride and Its Analogs” (Chapter 3) in Amiloride and Its Analogs, pp. 25-36, incorporated herein by reference. Other methods of preparing the compounds are described in, for example, U.S. Pat. No. 3,313,813, incorporated herein by reference. See in particular Methods A, B, C, and D described in U.S. Pat. No. 3,313,813. Other methods useful for the preparation of these compounds, especially for the preparation of the HNR³R⁴ fragment are described in, for example U.S. Pat. No. 6,903,105, U.S. Publication No. 2003/0199456 and U.S. Publication No. 2005/0090505, incorporated herein by reference in their entirety. Several assays may be used to characterize the compounds of the present invention. Representative assays are discussed below.
In Vitro Measure of Sodium Channel Blocking Activity and Reversibility

One assay used to assess mechanism of action and/or potency of the compounds of Formula I involves the determination of lumenal drug inhibition of airway epithelial sodium currents measured under short circuit current (I_{SC}) using airway epithelial monolayers mounted in Ussing chambers. Cells obtained from freshly excised human, dog or sheep airways are seeded onto porous 0.4 micron Snapwell™ Inserts (CoStar), cultured at air-liquid interface (ALI) conditions in hormonally defined media, and assayed for sodium transport activity (I_{SC} in μA/cm²) while bathed in Krebs Bicarbonate Ringer (KBR) in Ussing chambers. All test drug additions are to the lumenal bath with half-log dose addition protocols (from 1×10⁻¹¹ M to 3×10⁻⁵ M), and the cumulative change in I_{SC} (inhibition) recorded. All drugs are prepared in dimethyl sulfoxide as stock solutions at a concentration of 1×10⁻² M and stored at −20° C. Six preparations are typically run in parallel; one preparation per run incorporates a positive control. All data from the voltage clamps are collected via a computer interface and analyzed off-line.

Dose-effect relationships for all compounds are considered and analyzed by the Prism 3.0 program. EC₅₀ values, maximal effective concentrations are calculated and compared to positive controls.

In Vitro Durability of Sodium Channel Blockers: Surface Liquid Absorption, Transport, and Metabolic Profile

The airway bronchial epithelium is an absorptive epithelium (actively absorbs sodium and therefore water from the lumenal to serosal direction. Using a gravimetric (weighing) procedure, the lumenal surface liquid is weighed and changes recorded up to 36 h. An applied starting volume of buffer (modified Krebs-Henseleit Bicarbonate buffer solution) with and without equimolar concentrations of selected novel or commercially available sodium channel blockers are added to the starting buffer, and at selected time points the lumenal surface liquid mass is weighed and the mass recorded in mg. In addition, during the assay, samples are collected from both the surface liquid and serosal compartment, after which the wells re-weighted and weights recorded. The samples collected are analyzed using HPLC and or mass spectrometry, and the concentration of sodium channel blocker calculated, with any conjugate or metabolite noted.

Solubility of Compounds in Water or Sodium Chloride Solution

Compound solubility was measured in water, 0.12 or 0.9% sodium chloride solution at ambient temperature for up to 10 days. Using a UV/Visable Spectrophotometer and applying Beer's Law with the calculated extinction coefficient of amiloride (18.6 mM, absorbance values at 362 nm taken from D. Mazzo 1986) the free base concentration in solution was calculated at specified time points. All samples were stored for the duration of the experiment in a single/closure system consisting of glass vials with a stopper-top closure. The vials were maintained at ambient temperature, in the dark, and in the upright position. Compound stability was measured using reverse phase high performance liquid chromatography on the final filtered pulled sample (day 10).

Confocal Microscopy Assay of Amiloride Congener Uptake

Virtually all molecules studied fluoresce in the ultraviolet range. This property of these molecules may be used to directly measure cellular update using x-z confocal microscopy. Equimolar concentrations of experimental compounds and positive controls including amiloride and compounds that demonstrate rapid uptake into the cellular compartment (benzamil and phenamil) are placed on the apical surface of airway cultures on the stage of the confocal microscope. Serial x-z images are obtained with time and the magnitude of fluorescence accumulating in the cellular compartment is quantitated and plotted as a change in fluorescence versus time.

Pharmacological Effects and Mechanism of Action of the Drug in Animals

The effect of compounds for enhancing mucociliary clearance (MCC) can be measured using an in vivo model described by Sabater et al., Journal of Applied Physiology, 1999, pp. 2191-2196, incorporated herein by reference.

In Vivo Assay in Sheep—Methods

Animal Preparation: Adult ewes (ranging in weight from 25 to 35 kg) were restrained in an upright position in a specialized body harness adapted to a modified shopping cart. The animal's heads were immobilized and local anesthesia of the nasal passage was induced with 2% lidocaine. The animals were then nasally intubated with a 7.5 mm internal diameter endotracheal tube (ETT). The cuff of the ETT was placed just below the vocal cords and its position was verified with a flexible bronchoscope. After intubation the animals were allowed to equilibrate for approximately 20 minutes prior to initiating measurements of mucociliary clearance.

Administration of Radio-aerosol: Aerosols of ^{99m}Tc-Human serum albumin (3.1 mg/ml; containing approximately 20 mCi) were generated using a Raindrop Nebulizer which produces a droplet with a median aerodynamic diameter of 3.6 μm. The nebulizer was connected to a dosimetry system consisting of a solenoid valve and a source of compressed air (20 psi). The output of the nebulizer was directed into a plastic T connector; one end of which was connected to the endotracheal tube, the other was connected to a piston respirator. The system was activated for one second at the onset of the respirator's inspiratory cycle. The respirator was set at a tidal volume of 500 mL, an inspiratory to expiratory ratio of 1:1, and at a rate of 20 breaths per minute to maximize the central airway deposition. The sheep breathed the radio-labeled aerosol for 5 minutes. A gamma camera was used to measure the clearance of ^{99m}Tc-Human serum albumin from the airways. The camera was positioned above the animal's back with the sheep in a natural upright position supported in a cart so that the field of image was perpendicular to the animal's spinal cord. External radio-labeled markers were placed on the sheep to ensure proper alignment under the gamma camera. All images were stored in a computer integrated with the gamma camera. A region of interest was traced over the image corresponding to the right lung of the sheep and the counts were recorded. The counts were corrected for decay and expressed as percentage of radioactivity present in the initial baseline image. The left lung was excluded from the analysis because its outlines are superimposed over the stomach and counts can be swallowed and enter the stomach as radio-labeled mucus.

Treatment Protocol (Assessment of activity at t-zero): A baseline deposition image was obtained immediately after radio-aerosol administration. At time zero, after acquisition of the baseline image, vehicle control (distilled water), positive control (amiloride), or experimental compounds were aerosolized from a 4 ml volume using a Pari LC JetPlus nebulizer to free-breathing animals. The nebulizer was driven by compressed air with a flow of 8 liters per minute. The time to deliver the solution was 10 to 12 minutes. Animals were extubated immediately following delivery of the total dose in order to prevent false elevations in counts caused by aspiration of excess radio-tracer from the ETT. Serial images of the lung were obtained at 15-minute intervals during the first 2 hours after dosing and hourly for the next 6 hours after dosing for a total observation period of 8 hours. A washout period of at least 7 days separated dosing sessions with different experimental agents.

Treatment Protocol (Assessment of Activity at t-4 hours): The following variation of the standard protocol was used to assess the durability of response following a single exposure to vehicle control (distilled water), positive control compounds (amiloride or benzamil), or investigational agents. At time zero, vehicle control (distilled water), positive control (amiloride), or investigational compounds were aerosolized from a 4 ml volume using a Pari LC JetPlus nebulizer to free-breathing animals. The nebulizer was driven by compressed air with a flow of 8 liters per minute. The time to deliver the solution was 10 to 12 minutes. Animals were restrained in an upright position in a specialized body harness for 4 hours. At the end of the 4-hour period animals received a single dose of aerosolized ^{99m}Tc-Human serum albumin (3.1 mg/ml; containing approximately 20 mCi) from a Raindrop Nebulizer. Animals were extubated immediately following delivery of the total dose of radio-tracer. A baseline deposition image was obtained immediately after radio-aerosol administration. Serial images of the lung were obtained at 15-minute intervals during the first 2 hours after administration of the radio-tracer (representing hours 4 through 6 after drug administration) and hourly for the next 2 hours after dosing for a total observation period of 4 hours. A washout period of at least 7 days separated dosing sessions with different experimental agents.

Statistics: Data were analyzed using SYSTAT for Windows, version 5. Data were analyzed using a two-way repeated ANOVA (to assess overall effects), followed by a paried t-test to identify differences between specific pairs. Significance was accepted when P was less than or equal to 0.05. Slope values (calculated from data collected during the initial 45 minutes after dosing in the t-zero assessment) for mean MCC curves were calculated using linear least square regression to assess differences in the initial rates during the rapid clearance phase.

### EXAMPLES

Having generally described this invention, a further understanding can be obtained by reference to certain specific examples which are provided herein for purposes of illustration only and are not intended to be limiting unless otherwise specified.

Preparation of Sodium Channel Blockers

Materials and methods. All reagents and solvents were purchased from Aldrich Chemical Corp. and used without further purification. NMR spectra were obtained on either a Bruker WM 360 (¹H NMR at 360 MHz and ¹³C NMR at 90 MHz) or a Bruker AC 300 (¹H NMR at 300 MHz and ¹³C NMR at 75 MHz). Flash chromatography was performed on a Flash EluteJ system from Elution Solution (PO Box 5147, Charlottesville, Va. 22905) charged with a 90 g silica gel cartridge (40M FSO-0110-040155, 32-63 μm) at 20 psi (N₂). GC-analysis was performed on a Shimadzu GC-17 equipped with a Heliflex Capillary Column (Alltech); Phase: AT-1, Length: 10 meters, ID: 0.53 mm, Film: 0.25 micrometers. GC Parameters: Injector at 320° C., Detector at 320° C., ID gas flow: H₂ at 40 m/min., Air at 400 ml/min. Carrier gas: Split Ratio 16:1, N₂ flow at 15 m/min., N₂ velocity at 18 cm/sec. The temperature program is 70° C. for 0-3 min, 70-300° C. from 3-10 min, 300° C. from 10-15 min.

HPLC analysis was performed on a Gilson 322 Pump, detector UV/Vis-156 at 360 nm, equipped with a Microsorb MV C8 column, 100 A, 25 cm. Mobile phase: A=acetonitrile with 0.1% TFA, B=water with 0.1% TFA. Gradient program: 95:5 B:A for 1 min, then to 20:80 B:A over 7 min, then to 100% A over 1 min, followed by washout with 100% A for 11 min, flow rate: 1 ml/min.

Using these procedures, compounds according to Formula I may be prepared as shown in U.S. Ser. No. 10/920,391.

### Example 1

### PSA 24304

### Synthesis of 2-(4-{4-[N′-(3,5-diamino-6-chloropyrazine-2-carbonyl)guanidino]-butyl}phenoxy)-N-(3-dimethylaminopropyl)acetamide dimethanesulfonate

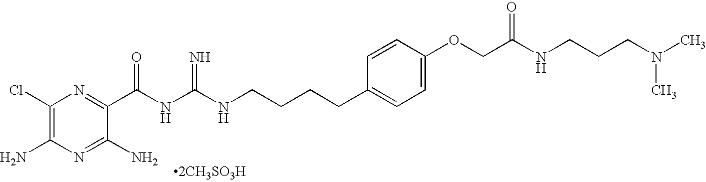 PSA 24304

### (4-{4-[(3-Dimethylaminopropylcarbamoyl)methoxy]phenyl}butyl)carbamic acid benzyl ester (3)

A solution of 1(0.50 g, 1.39 mmol) and CDI (0.25 g, 1.54 mmol) in THF (15 mL) was heated at 40° C. for 1 h. Then 2 (0.15 g, 1.47 mmol) was added into the reaction mixture at that temperature. The resulting solution was slowly cooled down to room temperature and further stirred at the temperature overnight. After that, the solvent was removed under reduced pressure and the residue was purified by Flash™ chromatography (BIOTAGE, Inc) (9:0.9:0.1 dichloromethane/methanol/concentrated ammonium hydroxide, v/v) to provide 3 (0.4 g, 61%) as a white solid. ¹H NMR (500 MHz, CD₃OD) δ 1.48 (m, 2H), 1.64 (m, 2H), 1.72 (m, 2H), 2.14 (s, 6H), 2.27 (m, 2H), 2.56 (m, 2H), 3.10 (m, 2H), 3.29 (m, 3H), 4.41 (s, 2H), 5.09 (s, 2H), 6.85 (d, 2H), 7.09 (d, 2H), 7.31 (m, 5H). m/z (ESI) 442.

### 2-[4-(4-Aminobutyl)phenoxy]-N-(3-dimethylaminopropyl)acetamide (4)

A suspension of 3 (377 mg, 0.85 mmol) and 10% palladium on carbon (0.30 g, 50% wet) in methanol (15 mL) was stirred at room temperature for 2 h under atmospheric pressure of hydrogen. The mixture was then filtered through a Celite pad and the solvent was evaporated to provide 4 (208 mg, 80%) as a white solid. The crude product was directly used in the next step without purification. ¹H NMR (300 MHz, CD₃OD) δ 1.61 (m, 6H), 2.11 (s, 6H), 2.32 (m, 2H), 2.65 (m, 4H), 3.28 (m, 2H), 4.45 (s, 2H), 6.90 (d, 2H), 7.19 (d, 2H). m/z (ESI) 308.

### 2-(4-{4-[N′-(3,5-Diamino-6-chloropyrazine-2-carbonyl)guanidino]butyl}phenoxy)-N-(3-dimethylaminopropyl)acetamide (6)

1-(3,5-Diamino-6-chloropyrazine-2-carbony)-2-methylisothiourea hydriodide (292 mg, 0.75 mmol) 5 was added to a solution of compound 4 (200 mg, 0.65 mmol), DIPEA (0.39 mL, 2.25 mmol), and ethanol (5 mL). The reaction mixture was stirred at 65° C. for 3 h. The solvent was removed under reduced pressure and the residue was purified by preparative TLC (80:18:2 dichloromethane/methanol/concentrated ammonium hydroxide, v/v) to provide 6 (190 mg, 56%) as a light yellow solid. ¹H NMR (300 MHz, CD₃OD) δ 1.65 (m, 6H), 2.15 (s, 6H), 2.32 (m, 2H), 2.64 (m, 2H), 3.27 (m, 8H), 4.49 (s, 2H), 6.89 (d, 2H), 7.15 (d, 2H). m/z (ESI) 520.

### 2-(4-{4-[N′-(3,5-Diamino-6-chloropyrazine-2-carbonyl)guanidino]butyl}phenoxy)-N-(3-dimethylaminopropyl)acetamide dimethanesulfonate (7)

Methanesulfonic acid (67.2 mg, 0.70 mmol) was added to the solution of 6 (182 mg, 0.35 mmol) in ethanol (4 mL). The resulting solution was stirred at room temperature for 0.5 h; then the solvent was completely evaporated, affording 212 mg (85%) of 7 as a light yellow solid. m.p. 187-190° C. ¹H NMR (300 MHz, CD₃OD) δ 1.65 (m, 4H), 1.90 (m, 2H), 2.67 (m, 8H), 2.85 (s, 6H), 3.15 (m, 2H), 3.40 (m, 4H), 4.49 (s, 2H), 6.89 (d, 2H), 7.15 (d, 2H). m/z (APCI) 520 [C₂₃H₃₄ClN₉O₃+H]⁺.

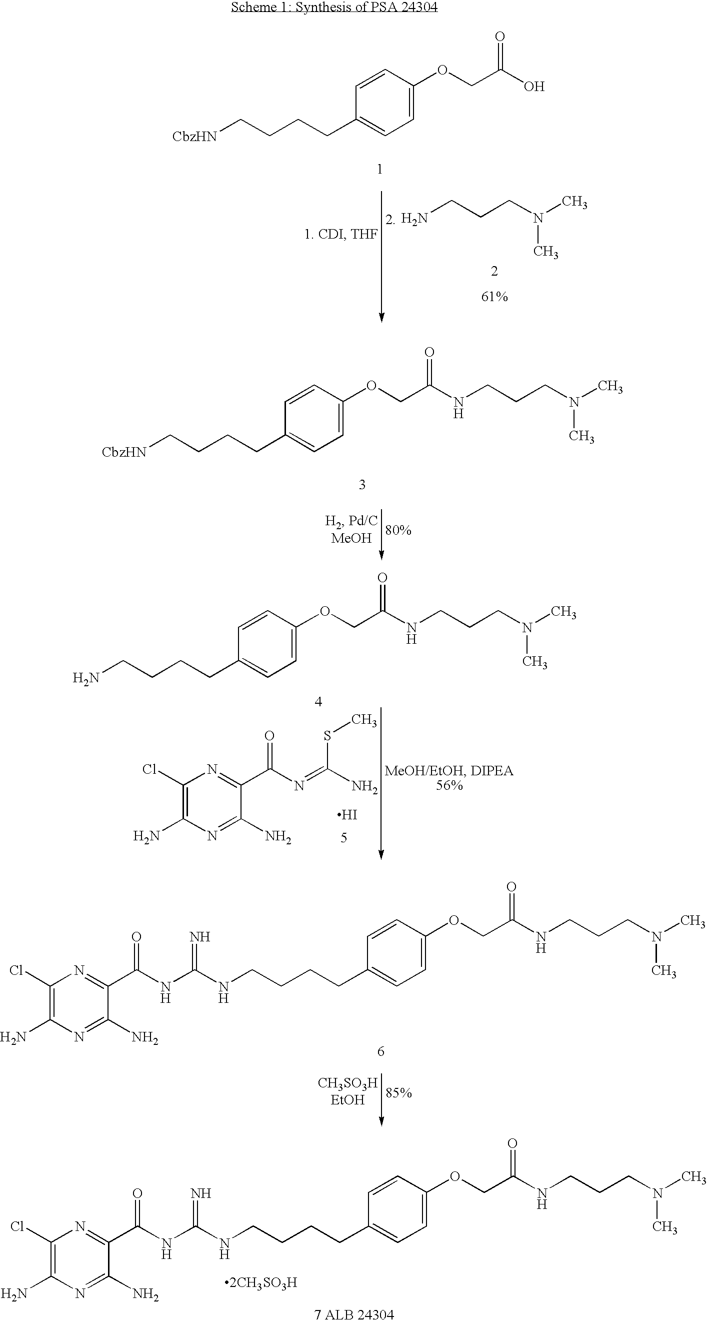

### Example 2

### Synthesis and Physical Properties of Selected Soluble Amides

Utilizing the procedures exemplified in Example 1 and Scheme 1, the compounds listed in Table 1 were prepared.

TABLE 1
Physical Properties of Selected Amides

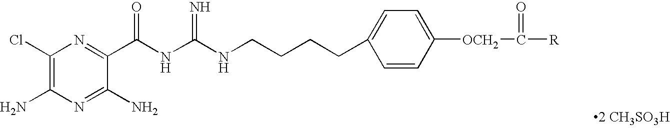

| | | Molecular | | Melting | | HPLC² | |
|---|---|---|---|---|---|---|---|
| | | Formula | Molecular | Point | | Analysis | |
| PSAI# | R = | 2CH₃SO₃H | Weight | ° C. | NMR¹ | (%) | M/Z³ |
| 23778 | —NH(CH₂)₂NH2 | C₂₀H₂₈ClN₉O₃ | 670.16 | 105-107° (d) | Consistent | 95.4 | 478 |
| 23185a | —NH(CH₂)₂N(CH₃)₂ | C₂₂H32ClN₉O₃ | 698.22 | 97-99° | Consistent | 97.4 | 506 |
| 24304 | NH(CH₂)₃N(CH₃)₂ | C₂₃H₃₄ClN₉O₃ | 712.25 | 187-190° | Consistent | 95.8 | 520 |
| 24305 | NH(CH₂)₄N(CH₃)₂ | C₂₄H₃₆ClN₉O₃ | 726.27 | 188-190° | Consistent | 97.0 | 534 |
| 23450 | NH(CH₂)₂N(CH₂CH₂OH)₂ | C₂₄H₃₆ClN₉O₅ | 758.27 | 87-89° | Consistent | 97.7 | 566 |
| 19913 | 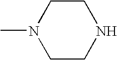 | C₂₂H₃₀ClN₉O₃ | 696.21 | 72-74° | Consistent | 97.9 | 504 |

Notes:
¹NMR = 500 MHz ¹H NMR Spectrum (CD₃OD).
²HPLC—Polarity dC18 Column, Detector @ 200 nM.
³M/Z = APCI Mass Spectrum [Free Base + H]⁺.

### Example 3

### Solubility of Selected Amides

Table 2 gives the solubility in saline of selected amide bis methane sulfonic acid salts and compares them to the mono addition methane sulfonic acid salt of PSA 9714.

TABLE 2
Solubility of Selected Amides

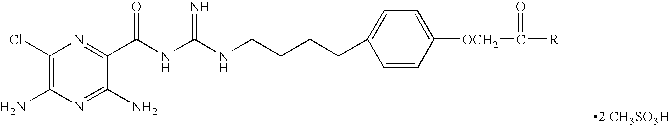

| PSA# | R = | S¹ | S² |
|---|---|---|---|
| 9714 | —NH₂ | <0.1 mg/ml | <0.1 mg/ml |
| 237778 | —NH(CH₂)₂NH₂ | >5 mg/ml | |
| 23185a | —NH(CH₂)₂N(CH₃)₂ | >5 mg/ml | |
| 24304 | —NH(CH₂)₃N(CH₃)₂ | >5 mg/ml | |
| 24305 | —NH(CH₂)₄N(CH₃)₂ | >5 mg/ml | >5 mg/ml |
| 23450 | NH(CH₂)₂N(CH₂CH₂OH)₂ | >5 mg/ml | >5 mg/ml |
| 19913 | 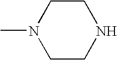 | >5 mg/ml | |

S¹ = Solubility in 0.12% NaCl Solution
S² = Solubility in 0.9% NaCl Solution (normal Saline)

### Example 4

### Sodium Channel Blocking Activity of Selected Soluble Amides

Utilizing the tests set forth above, Table 3 summaries the ENaC blocking ability of some of the amides of this invention when assayed in canine bronchial epithelium.

TABLE 3
Epithelial Sodium Channel Blocking Activity of Selected Amides

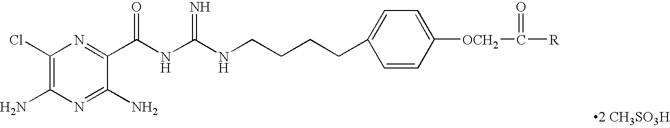

| PSA# | R = | IC₅₀ (nM) | Fold Amiloride** (PSA4022 = 100) |
|---|---|---|---|
| 9714 | —NH₂ | 15.2 + 6.3 (n = 36) | 62 |
| 237778 | —NH(CH₂)₂NH₂ | 5 ± 2 (n = 4) | 146 |
| 23185a | —NH(CH₂)₂N(CH₃)₂ | 14 ± 9 (n = 3) | 59 |
| 24304 | —NH(CH₂)₃N(CH₃)₂ | 4 ± 2 (n = 7) | 173 |
| 24305 | —NH(CH₂)₄N(CH₃)₂ | 6 ± 5 (n = 6) | 152 |
| 23450 | NH(CH₂)₂N(CH₂CH₂OH)₂ | 18 | 38 |
| 19913 | 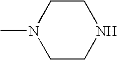 | 9 ± 1 (n = 2) | 98 |

**Relative potency for PSA4022 = 100 using IC₅₀ from PSA4O22 in same run.

### Example 5

### N-(3,5-Diamino-6-chloropyrazine-2-carbonyl)-N′-{4-[4-(piperazine-1-carbonyl)phenyl]butyl}guanidine bis-methanesulfonate (PSA23607)

### 4-(4-Aminobutyl)benzoic acid (8)

A solution of sodium hydroxide (0.69 g, 17.37 mmol) in water (30 mL) was added to a solution of 24 (1.2 g, 5.79 mmol) in methanol (30 mL) and stirred at room temperature for 48 h. Then the solvent was removed under reduced pressure. Water (20 mL) was added and pH was adjusted to 7 with HCl. The white solid precipitate was filtered off, washed with water and dried in vacuum. The crude product 8 (1.39 g) was obtained as a white solid and used for the next step without further purification.

### 4-(4-Benzyloxycarbonylaminobutyl)benzoic acid (9)

Sodium hydrogencarbonate (0.95 g, 11.32 mmol) was added into a suspension of 30 in THF (120 mL), followed by water (10 mL), affording a clear solution. Benzyl chloroformate (1.21 mL, 8.49 mmol) was then added into the reaction mixture at 0° C. The reaction mixture was then stirred at room temperature overnight. After that, the solvent was removed under reduced pressure. Ethyl acetate (70 mL) was added to the residue and the solution was washed with 2N HCl (2×30 mL) and water (2×50 mL), then dried in vacuum. 1.82 g (98%) of 9 was obtained as a white solid. ¹H NMR (300 MHz, DMSO-d₆) δ 1.11 (m, 2H), 1.28 (m, 2H), 2.33 (m, 2H), 3.02 (m, 2H), 5.01 (m, 2H), 7.15 (m, 7H), 7.93 (d 2H).

### N-(3,5-Diamino-6-chloropyrazine-2-carbonyl)-N′-{4-[4-(piperazine-1-carbonyl)phenyl]butyl}guanidine bis-methanesulfonate (PSA23607)

Compound 9 was converted to PSA236507 utilizing the general methods described in Example 1 yielding the desired product, melting point 122-124° C., 500 MHz ¹H NMR Spectrum (CD₃OD) was consistent with assigned structure, HPLC Analysis 98.5% (area percent), Polarity dC18 Column, Detector @ 220 nm, ESI mass Spectrum m/z 474 [C₂₁H₂₈ClN₉O₂+H]⁺. PSA23607 had an IC₅₀ of 12.5±0.5 nM on canine bronchial epithelial and had a solubility of greater than 5 mg/ml in 0.12% saline solution.

### Example 6

### 3-[4-(4-Aminobutyl)phenoxy]-2-tert-butoxycarbonylaminopropionic acid methyl ester (13)

### 3-[4-(4-Benzyloxycarbonylaminobutyl)phenoxy]-2-(tritylamino)propionic acid methyl ester (10)

Commercially available N-trityl-L-serine methyl ester (1.60 g, 5.34 mmol) was combined with triphenylphosphine (1.28 g, 4.88 mmol) and [4-(4-hydroxyphenyl)butyl]carbamic acid benzyl ester (2.0 g, 6.68 mmol) in benzene (40 mL) at room temperature. Diisopropyl azodicarboxylate (0.958 mL, 4.86 mmol) was added dropwise and the reaction was stirred for 14 days. The solvent was removed under reduced pressure and the residue was purified by column chromatography (silica gel, eluent: 6:1, v/v dichloromethane/ethyl acetate) to provide compound 10 (2.22 g, 51%). ¹H NMR (300 MHz, CDCl₃) δ 7.52 (m, 6H), 7.39-7.14 (m, 14H), 7.06 (d, 2H), 6.79 (d, 2H), 5.09(s, 2H), 4.72 (m, 1H), 4.24 (m, 1H), 4.01 (m, 1H), 3.72 (m, 1H), 3.22 (s, 3H), 3.18 (m, 2H), 2.88 (d, 1H), 2.57 (m, 2H), 1.66-1.48 (m, 4H). R_{f}=0.91 (5:1 v/v dichloromethane/ethyl acetate).

### 3-[4-(4-Benzyloxycarbonylaminobutyl)phenoxy]-2-tert-butoxycarbonylamino-propionic acid methyl ester (12)

Compound 10 (2.22 g, 3.45 mmol) was dissolved in a mixture of dichloromethane/water (25 mL/0.5 mL) then trifluoroacetic acid (0.75 mL, 10.0 mmol) was added and the reaction was stirred for 2 h. The solvent was removed under reduced pressure and the residue was dissolved in dichloromethane (25 mL) and treated with triethylamine (0.72 mL, 5.12 mmol) and di-tert-butyl dicarbonate (0.829 g, 3.79 mmol) for 72 h. Removal of the solvents under reduced pressure followed by column chromatography (silica gel, eluent: 9:1, v/v dichloromethane/ethyl acetate) provided compound 12 (0.90 g, 52%). ¹H NMR (300 MHz, CDCl₃) δ 7.34 (m, 5H), 7.07 (d, 2H), 6.79 (d, 2H), 5.50 (d, 1H), 5.10 (s, 2H), 4.68 (m, 2H); 4.38 (m, 1H), 4.17 (m, 1H), 3.77 (s, 3H), 3.20 (m, 2H), 2.57 (m, 2H), 1.67-1.48 (m, 4H), 1.45 (s, 9H). m/z (APCI) 401 [C₂₇H₃₆N₂O₇—Boc+H]⁺.

### 3-[4-(4-Aminobutyl)phenoxy]-2-tert-butoxycarbonylaminopropionic acid methyl ester (13)

Compound 12 (505 mg, 1.00 mmol) was dissolved in methanol (20 mL) and 10% palladium on carbon (100 mg) was added. The flask was evacuated, filled with hydrogen gas under balloon pressure and stirred overnight. Filtration through celite to remove the catalyst followed by removal of the solvent under reduced pressure provided compound 13 (366 mg, 98%). ¹H NMR (300 MHz, CDCl₃) δ 7.08 (d, 2H), 6.80 (d, 2H), 5.51 (d, 1H), 4.66 (m, 1H), 4.38 (m, 1H), 4.17 (m, 1H), 3.78 (s, 3H), 2.73 (m, 2H), 2.58 (m, 2H), 1.90 (br s, 2H), 1.62 (m, 2H), 1.50 (m, 2H), 1.48 (s, 9H).

### Example 7

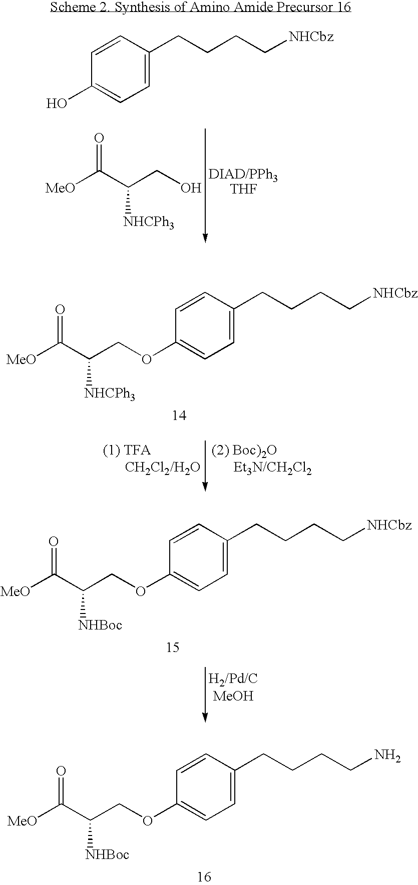

### 3-[4-(4-Aminobutyl)phenoxy]-2-tert-butoxycarbonylaminopropionic acid methyl ester (16)

### 3-[4-(4-Benzyloxycarbonylaminobutyl)phenoxy]-2-(tritylamino)propionic acid methyl ester (14)

Commercially available N-trityl-L-serine methyl ester (1.60 g, 5.34 mmol) was combined with triphenylphosphine (1.28 g, 4.88 mmol) and [4-(4-hydroxyphenyl)butyl]carbamic acid benzyl ester (2.0 g, 6.68 mmol) in benzene (40 mL) at room temperature. Diisopropyl azodicarboxylate (0.958 mL, 4.86 mmol) was added dropwise and the reaction was stirred for 14 days. The solvent was removed under reduced pressure and the residue was purified by column chromatography (silica gel, eluent: 6:1, v/v dichloromethane/ethyl acetate) to provide compound 141 (2.22 g, 51%). ¹H NMR (300 MHz, CDCl₃) δ 7.52 (m, 6H), 7.39-7.14 (m, 14H), 7.06 (d, 2H), 6.79 (d, 2H), 5.09(s, 2H), 4.72 (m, 1H), 4.24 (m, 1H), 4.01 (m, 1H), 3.72 (m, 1H), 3.22 (s, 3H), 3.18 (m, 2H), 2.88 (d, 1H), 2.57 (m, 2H), 1.66-1.48 (m, 4H). R_{f}=0.91 (5:1 v/v dichloromethane/ethyl acetate).

### 3-[4-(4-Benzyloxycarbonylaminobutyl)phenoxy]-2-tert-butoxycarbonylamino-propionic acid methyl ester (15)

Compound 14 (2.22 g, 3.45 mmol) was dissolved in a mixture of dichloromethane/water (25 mL/0.5 mL) then trifluoroacetic acid (0.75 mL, 10.0 mmol) was added and the reaction was stirred for 2 h. The solvent was removed under reduced pressure and the residue was dissolved in dichloromethane (25 mL) and treated with triethylamine (0.72 mL, 5.12 mmol) and di-tert-butyl dicarbonate (0.829 g, 3.79 mmol) for 72 h. Removal of the solvents under reduced pressure followed by column chromatography (silica gel, eluent: 9:1, v/v dichloromethane/ethyl acetate) provided compound 15 (0.90 g, 52%). ¹H NMR (300 MHz, CDCl₃) δ 7.34 (m, 5H), 7.07 (d, 2H), 6.79 (d, 2H), 5.50 (d, 1H), 5.10 (s, 2H), 4.68 (m, 2H), 4.38 (m, 1H), 4.17 (m, 1H), 3.77 (s, 3H), 3.20 (m, 2H), 2.57 (m, 2H), 1.67-1.48 (m, 4H), 1.45 (s, 9H). m/z (APCI) 401 [C₂₇H₃₆N₂O₇—Boc+H]⁺.

### 3-[4-(4-Aminobutyl)phenoxy]-2-tert-butoxycarbonylaminopropionic acid methyl ester (16)

Compound 15 (505 mg, 1.00 mmol) was dissolved in methanol (20 mL) and 10% palladium on carbon (100 mg) was added. The flask was evacuated, filled with hydrogen gas under balloon pressure and stirred overnight. Filtration through celite to remove the catalyst followed by removal of the solvent under reduced pressure provided compound 16 (366 mg, 98%). ¹H NMR (300 MHz, CDCl₃) δ 7.08 (d, 2H), 6.80 (d, 2H), 5.51 (d, 1H), 4.66 (m, 1H), 4.38 (m, 1H), 4.17 (m, 1H), 3.78 (s, 3H), 2.73 (m, 2H), 2.58 (m, 2H), 1.90 (br s, 2H), 1.62 (m, 2H), 1.50 (m, 2H), 1.48 (s, 9H).

### Example 8

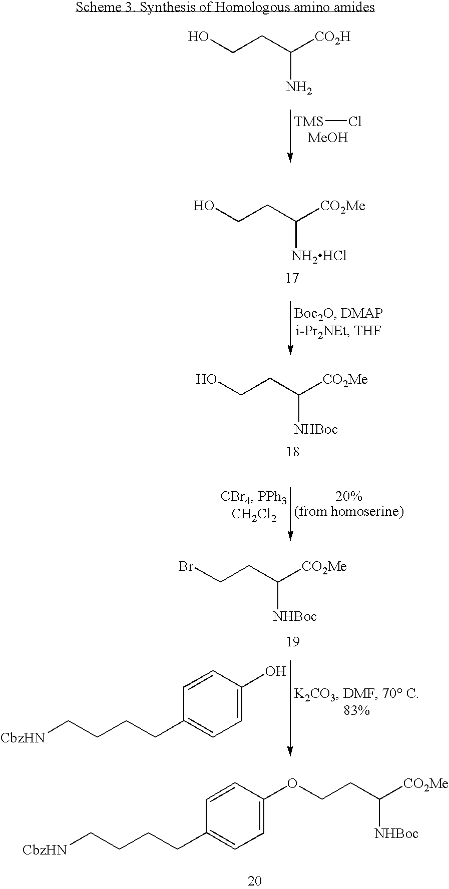

### {4-[4-(3-tert-Butoxycarbonylamino-3-carbamoylpropoxy)phenyl]butyl}carbamic acid benzyl ester (20)

### 2-Amino-4-hydroxybutyric acid methyl ester hydrochloride (17)

A suspension of DL-homoserine (1.00 g, 8.39 mmol) in methanol (40 mL) was placed in an ice bath. Trimethylsilyl chloride (2.34 mL, 18.5 mmol) was added dropwise via syringe. The reaction mixture gradually became homogenous and was further stirred at rt for 14 h, concentrated by rotary evaporation, and further dried under high vacuum. The crude oil thus obtained was used for the next step without further purification. ¹H NMR (300 MHz, CD₃OD) δ 2.00-2.24 (m, 2H), 3.70-3.80 (m, 2H), 3.85 (s, 3H), 4.12-4.22 (m, 1H). m/z (ESI) 134 [C₅H₁₁NO₃+H]⁺.

### 2-tert-Butoxycarbonylamino-4-hydroxybutyric acid methyl ester (18)

2-Amino-4-hydroxybutyric acid methyl ester hydrochloride (17) was suspended in anhydrous THF (15 mL) and placed in an ice bath. Diisopropylethylamine (2.92 mL, 16.8 mmol) was added via syringe, followed by the addition of DMAP (205 mg, 1.68 mmol) and Boc₂O (3.85 g, 17.6 mmol). The mixture was stirred at 0° C. for 10 min and at room temperature for 14 h. Solvent was removed under reduced pressure and residue was taken up by ethyl acetate (100 mL), washed with water (30 mL×2) and brine (40 mL), dried over sodium sulfate, and concentrated. The colorless oil (1.96 g) was used for the next step without further purification. ¹H NMR (300 MHz, CDCl₃) δ 1.45 (s, 9H), 2.05-2.28 (m, 2H), 3.68-3.75 (m, 2H), 3.80 (s, 3H), 4.08-4.15 (m, 1H), 5.30-5.41 (m, 1H). m/z (ESI) 234 [C₁₀H₁₉NO₅+H]⁺.

### 4-Bromo-2-tert-butoxycarbonylaminobutyric acid methyl ester (19)

A solution of triphenylphosphine (2.20 g, 8.39 mmol) in dry CH₂Cl₂ (20 mL) was added dropwise via syringe to a solution of N-Boc homoserine methyl ester 18 (8.39 mmol) and carbon tetrabromide (4.18 g, 12.60 mmol) in dry CH₂Cl₂ (20 mL). The resulting dark solution was stirred at room temperature for 16 h. Hexanes was added and precipitates were removed by suction filtration. The filtrate was concentrated under reduced pressure and subject to flash silica gel column chromatography using ethyl acetate/hexanes (1:10, v/v then 1:6, v/v) to give the desired product 19 as a yellow oil (501 mg, 20% overall yield from homoserine). ¹H NMR (300 MHz, CDCl₃) δ 1.45 (s, 9H), 2.12-2.48 (m, 2H), 3.39-3.47 (m, 2H), 3.78 (s, 3H), 4.33-4.48 (m, 1H), 5.10-5.22 (m, 1H). m/z (ESI) 296 [C₁₀H₁₈BrNO₄+H]⁺.

### 4-[4-(4-Benzyloxycarbonylaminobutyl)phenoxy]-2-tert-butoxycarbonylamino-butyric acid methyl ester (20)

Potassium carbonate (935 mg, 6.77 mmol) was added in one portion to a solution of 4-[4-(benzyloxycarbonylamino)butyl]phenol (506 mg, 1.69 mmol) and N-Boc bromide 19 (501 mg, 1.69 mmol) in anhydrous DMF (10 mL). The reaction mixture was stirred at 70° C. (oil bath) for 14 h, cooled to room temperature, and diluted with ethyl acetate (100 mL) and hexanes (20 mL). The mixture was washed with water (4×20 mL) and brine (40 mL) and concentrated under reduced pressure. Flash silica gel column chromatography using ethyl acetate/CH₂Cl₂ (1:25, 1:20, v/v) gave the desired product 20 as a thick yellow oil (718 mg, 83% yield). ¹H NMR (300 MHz, CDCl₃) δ 1.44 (s, 9H), 1.48-1.68 (m, 4H), 2.12-2.38 (m, 2H), 2.48-2.60 (m, 2H), 3.10-3.24 (m, 2H), 3.75 (s, 3H), 3.97-4.06 (m, 2H), 4.41-4.52 (m, 1H); 4.70 (br s, 1H), 5.09 (s, 2H), 5.25-5.37 (m, 1H), 6.70-6.80 (m, 2H), 6.95-7.09 (m, 2H), 7.30-7.38 (m, 5H). m/z (ESI) 515 [C₂₈H₃₈N₂O₇+H]⁺.

### Example 9

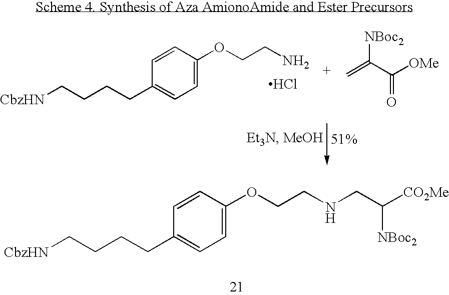

### 3-{2-[4-(4-Benzyloxycarbonylaminobutyl)phenoxy]ethylamino}-2-N,N′-di-(tert-butoxycarbonyl)aminopropionic acid methyl ester (21)

A mixture of {4-[4-(2-aminoethoxy)phenyl]butyl}carbamic acid benzyl ester hydrochloride (141 mg, 0.372 mmol), 2-[N,N′-di(tert-butoxycarbonyl)]aminoacrylic acid methyl ester (102 mg, 0.338 mmol), and triethylamine (0.16 mL, 1.11 mmol) in methanol (3 mL) was stirred at 55° C. (oil bath) for 16 h. It was then cooled to room temperature. The solvent was removed by rotary evaporation. The residue was taken up in ethyl acetate and washed with saturated sodium bicarbonate solution and brine. The organic layer was concentrated in vacuo and purified by flash silica gel column chromatography using ethyl acetate/hexanes (gradient 1:10, 1:6, 1:4, and 1:2, v/v) to give the desired Michael adduct 21 (110 mg, 51% yield). ¹H NMR (300 MHz, CDCl₃) δ 1.42 (s, 9H), 1.43-1.50 (m, 9H), 1.50-1.65 (m, 4H), 2.50-2.60 (m, 2H), 3.12-3.25 (m, 2H), 3.49-3.72 (m, 4H), 3.75 (s, 3H), 3.98-4.10 (m, 2H), 4.45-4.65 (m, 1H), 4.79 (br s, 1H), 5.09 (s, 2H), 5.50 (m, 1H), 6.81 (d, J=8.5 Hz, 2H), 7.06 (d, J=8.5 Hz, 2H), 7.28-7.38 (m, 5H). m/z (ESI) 644 [C₃₄H₄₉N₃O₉+H]⁺.

### Example 10

PSA 1

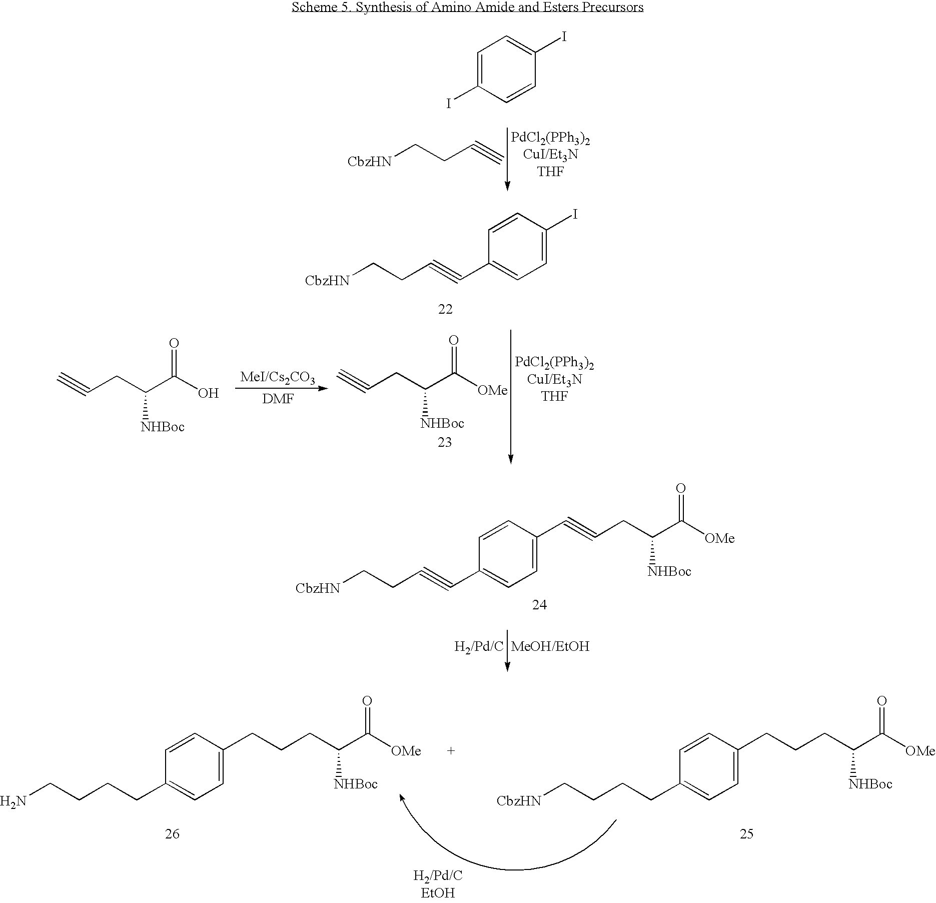

### [4-(4-Iodophenyl)but-3-ynyl]carbamic acid benzyl ester (22)

To a mixture of anhydrous THF and triethylamine (24 mL, 2/1, v/v) were sequentially added 1,4-diiodobenzene (2.03 g, 6.15 mmol) and copper (I) iodide (0.094 g, 0.246 mmol). The mixture was stirred at room temperature for 15 min. The flask was then evacuated and re-filled with Argon. The procedure was repeated three more times to ensure no oxygen remained. The catalyst, dichlorobis(triphenylphosphine)palladium(II) (0.173 g, 0.246 mmol) was added into the mixture under Argon protection. The other starting material, but-3-ynylcarbamic acid benzyl ester (0.50 g, 2.46 mmol), dissolved in THF (8 mL) was added dropwise over 6 hours. The newly formed reaction mixture was further stirred at room temperature overnight. The solid in the reaction mixture was vacuum filtered. The filtrate was concentrated. The residue was re-dissolved in dichloromethane and purified by column chromatography, eluting with a mixture of ethyl acetate (0-12%) and hexanes (100-88%) to afford the product 22 (0.852 g, 86%) as an off-white solid. ¹H NMR (300 MHz, CDCl₃): δ 2.61 (t, J=6.4 Hz, 2H), 3.44 (t, J=6.4 Hz, 2H), 5.07 (br s, 1H), 5.12 (s, 2H), 7.10 (d, J=8.3 Hz, 2H), 7.35 (m, 5H), 7.63 (d, J=8.3 Hz, 2H). m/z (APCI) 405 [C₁₈H₁₆NO₂+H]⁺.

### 2-(R)-tert-Butoxycarbonylaminopent-4-ynoic acid methyl ester (23)

The commercially available compound, 2-(R)-tert-butoxycarbonylaminopent-4-ynoic acid (0.321 g, 1.50 mmol) was dissolved in anhydrous DMF (5 mL). To the solution was added cesium carbonate (0.54 g, 1.65 mmol) in one portion. The mixture was stirred at room temperature for 45 min before methyl iodide (0.20 mL, 3.00 mmol) was added, and further stirred for three hours. The reaction was quenched with water (10 mL). The organics was extracted with dichloromethane (2×30 mL), washed with water (3×50 mL), and dried over anhydrous sodium sulfate. The solvent was completely removed under vacuum. The residue was further dried under high vacuum over night and used in the next reaction without further purification. The product 23 was obtained as a colorless viscous oil (0.326 g, 95% yield). ¹H NMR (300 MHz, CDCl₃): δ 1.48 (s, 9H), 2.08 (t, J=2.6 Hz, 1H), 2.75 (t, J=6.3 Hz, 2H), 3.78 (s, 3H), 4.46 (m, 1H), 5.32 (br s, 5H).

### 5-[4-(4-Benzyloxycarbonylaminobut-1-ynyl)phenyl]-2-(R)-tert-butoxycarbonylaminopent-4-ynoic acid methyl ester (24)

Compound 22, [4-(4-iodophenyl)-but-3-ynyl]carbamic acid benzyl ester (0.52 g, 1.283 mmol) was dissolved in a mixture of anhydrous THF and triethylamine (8 mL, 1/1, v/v). To the solution was added copper (I) iodide (0.025 g, 0.128 mmol). The mixture was stirred at room temperature for 15 min. The flask was then evacuated and re-filled with Argon. The procedure was repeated three more times to ensure no oxygen remained. The catalyst, dichlorobis(triphenylphosphine)palladium(II) (0.09 g, 0.128 mmol) was added into the mixture under Argon protection. The mixture was further stirred at room temperature for 30 min. The other starting material 23, 2-tert-butoxycarbonylamino-pent-4-ynoic acid methyl ester (0.32 g, 1.412 mmol), dissolved in THF (4 mL) was added dropwise over 15 min. The newly formed reaction mixture was further stirred overnight at room temperature. The solid in the reaction mixture was vacuum filtered. The filtrate was concentrated. The residue was re-dissolved in dichloromethane and purified by column chromatography, eluting with a mixture of ethyl acetate (0-25%) and hexanes (100-75%) to afford the product 24 (0.64 g, 99%) as a gummy, yellowish solid. ¹H NMR (300 MHz, CDCl₃): δ 1.46 (s, 9H), 2.64 (t, J=6.3 Hz, 2H), 2.95 (t, J=4.1 Hz, 2H), 3.42 (m, 2H), 3.79 (s, 3H), 4.56 (m, 1H), 5.12 (s, 2H), 5.38 (br s, 1H), 7.29 (s, 4H), 7.35 (m, 5H). m/z (APCI) 502 [C₂₉H₃₂N₂O₆—H]⁺.

### 5-[4-(4-Aminobutyl)phenyl]-2-(R)-tert-butoxycarbonylaminopentynoic acid methyl ester (26)

Compound 24, 5-[4-(4-benzyloxycarbonylaminobut-1-ynyl)phenyl]-2-(R)-tert-butoxycarbonylaminopent-4-ynoic acid methyl ester (0.36 g, 0.713 mmol) was dissolved in a mixture of ethanol and methanol (50 mL, 1/1, v/v) and placed in a Parr shaker bottle. To the solution was added 10% Palladium on carbon (0.20 g, wet) in one portion under Argon protection. The flask was evacuated and re-filled with Argon. The procedure was repeated three more times. The mixture was then stirred at room temperature over night under 35 psi hydrogen atmosphere. The flask was then evacuated and re-filled with nitrogen. The procedure was repeated three times. The catalyst was then filtered under vacuum and washed with ethanol (2×5 mL). The filtrate and washings were combined and concentrated under reduced pressure. The residue was chromatographed over silica gel, eluting with a mixture of methanol (0-12%), ammonium hydroxide (0-1.2%) and di-chloromethane (100-86.8%), to afford two products, the desired product 226 (0.045 g, 17%, a colorless, glass-like solid) and its protected form 25 (0.218 m, 60%, a yellowish solid). ¹H NMR (300 MHz, CD₃OD) for compound 26: δ 1.43 (s, 9H), 1.63-1.76 (m, 8H), 2.58-2.64 (m, 4H), 2.79 (t, J=7.0 Hz, 2H), 3.69 (s, 3H), 4.10 (m, 1H), 7.09 (s, 4H). m/z (APCI) for compound 7: 379 [C₁₂H₃₄N₂O₄+H]⁺.

¹H N (300 MHz, CD₃OD) for compound 25: δ 1.42 (s, 9H), 1.61-1.78 (m, 8H), 2.54-2.68 (m, 4H), 2.92 (t, J=7.0 Hz, 2H), 3.14 (t, J=6.9 Hz, 2H), 3.70 (s, 3H), 4.13 (m, 1H), 5.18 (s, 2H), 7.08 (s, 4H), 7.35 (m, 5H).

The compound 25 was resubmitted to hydrogenation to remove the benzyloxycarbonyl protecting group. The procedure was performed as follows: the compound 25 (0.218 g, 0.425 mmol) was dissolved in ethanol (10 mL). The solution was purged with nitrogen both before and after the palladium catalyst (0.10 g, 10% on charcoal, 50% wet) was added, and subjected to hydrogenation for two hours under atmospheric hydrogen. The catalyst was vacuum filtered and washed with ethanol (2×5 mL). The filtrate and washings were combined and concentrated under vacuum. The residue was chromatographed over silica gel, eluting with a mixture of methanol (0-14%), ammonium hydroxide (0-1.4%) and dichloromethane (100-84.6%), to afford the compound 26 (0.131 g, 81%).

### Example 11

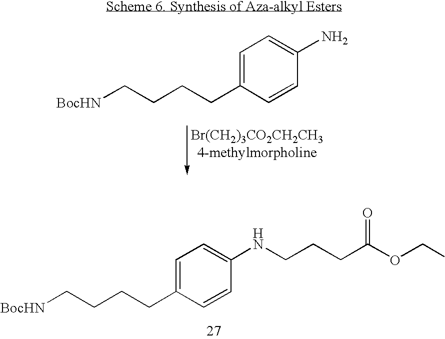

### 4-[4-(4-tert-Butoxycarbonylaminobutyl)phenylamino]butyric acid ethyl ester (27)

A solution of [4-(4-aminophenyl)butyl]carbamic acid tert-butyl ester (1.7 g, 6.43 mmol), 4-bromo-n-butyric acid ethyl ester (1.88 g, 9.64 mmol), 4-methylmorpholine (1.0 mL, 9.64 mmol), and DMF (10 mL) was stirred at 85° C. for 3 hours under a nitrogen atmosphere. The solvent was evaporated in vacuo. The residue was purified by flash chromatography (silica gel, 4:1, hexanes/ethyl acetate, v/v) to provide 27 (0.44 g, 18%) as a colorless oil. ¹H NMR (300 MHz, CDCl₃) δ 1.23 (t, 3H), 1.47 (s, 9H), 1.55 (m, 4H), 1.96 (m, 2H), 2.43 (t, 2H), 2.51 (t, 2H), 3.19 (m, 4H), 4.14 (q, 2H), 6.54 (d, 2H), 7.00 (d, 2H). m/z (ESI) 379.

### Example 12

### Synthesis of Amino Amides

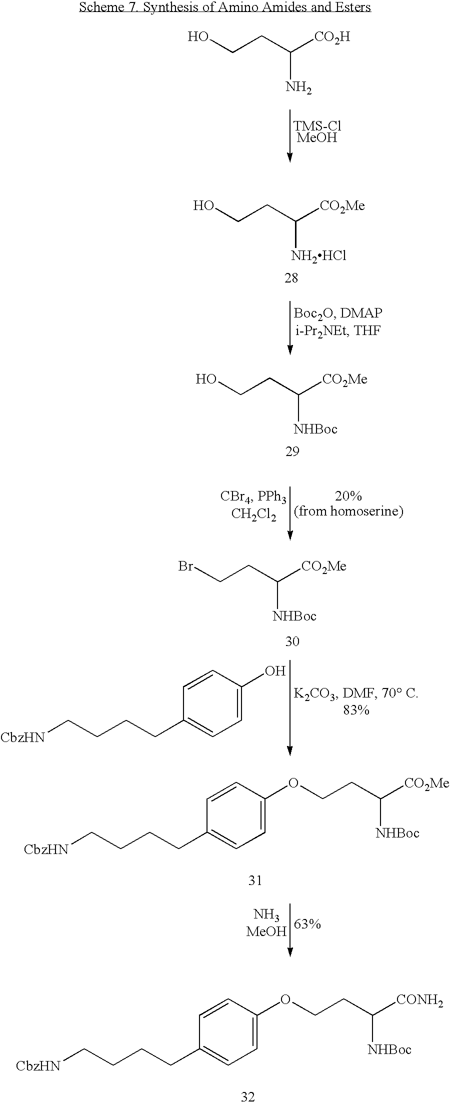

### Synthesis of {3-[4-(4-Aminobutyl)phenoxy]-1-carbamoylpropyl}carbamic acid tert-butyl ester (32)

### 2-Amino-4-hydroxybutyric acid methyl ester hydrochloride (28)

A suspension of DL-homoserine (1.00 g, 8.39 mmol) in methanol (40 mL) was placed in an ice bath. Trimethylsilyl chloride (2.34 mL, 18.5 mmol) was added dropwise via syringe. The reaction mixture gradually became homogenous and was further stirred at rt for 14 h, concentrated by rotary evaporation, and further dried under high vacuum. The crude oil thus obtained was used for the next step without further purification. ¹H NMR (300 MHz, CD₃OD) δ 2.00-2.24 (m, 2H), 3.70-3.80 (m, 2H), 3.85 (s, 3H), 4.12-4.22 (m, 1H). m/z (ESI) 134 [C₅H, NO₃+H]⁺.

### 2-tert-Butoxycarbonylamino-4-hydroxybutyric acid methyl ester (29)

2-Amino-4-hydroxybutyric acid methyl ester hydrochloride (28) was suspended in anhydrous THF (15 mL) and placed in an ice bath. Diisopropylethylamine (2.92 mL, 16.8 mmol) was added via syringe, followed by the addition of DMAP (205 mg, 1.68 mmol) and Boc₂O (3.85 g, 17.6 mmol). The mixture was stirred at 0° C. for 10 min and at room temperature for 14 h. Solvent was removed under reduced pressure and residue was taken up by ethyl acetate (100 mL), washed with water (30 mL×2) and brine (40 mL), dried over sodium sulfate, and concentrated. The colorless oil (1.96 g) was used for the next step without further purification. ¹H NMR (300 MHz, CDCl₃) δ 1.45 (s, 9H), 2.05-2.28 (m, 2H), 3.68-3.75 (m, 2H), 3.80 (s, 3H), 4.08-4.15 (m, 1H), 5.30-5.41 (m, 1H). m/z (ESI) 234 [C₁₀H₁₉NO₅+H]⁺.

### 4-Bromo-2-tert-butoxycarbonylaminobutyric acid methyl ester (30)

A solution of triphenylphosphine (2.20 g, 8.39 mmol) in dry CH₂Cl₂ (20 mL) was added dropwise via syringe to a solution of N-Boc homoserine methyl ester 29 (8.39 mmol) and carbon tetrabromide (4.18 g, 12.60 mmol) in dry CH₂Cl₂ (20 mL). The resulting dark solution was stirred at room temperature for 16 h. Hexanes was added and precipitates were removed by suction filtration. The filtrate was concentrated under reduced pressure and subject to flash silica gel column chromatography using ethyl acetate/hexanes (1:10, v/v then 1:6, v/v) to give the desired product 30 as a yellow oil (501 mg, 20% overall yield from homoserine). ¹H NMR (300 MHz, CDCl₃) δ 1.45 (s, 9H), 2.12-2.48 (m, 2H), 3.39-3.47 (m, 2H), 3.78 (s, 3H), 4.33-4.48 (m, 1H), 5.10-5.22 (m, 1H). m/z (ESI) 296 [C₁₀H₁₈BrNO₄+H]⁺.

### 4-[4-(4-Benzyloxycarbonylaminobutyl)phenoxy]-2-tert-butoxycarbonylamino-butyric acid methyl ester (31)

Potassium carbonate (935 mg, 6.77 mmol) was added in one portion to a solution of 4-[4-(benzyloxycarbonylamino)butyl]phenol (506 mg, 1.69 mmol) and N-Boc bromide 30 (501 mg, 1.69 mmol) in anhydrous DMF (10 mL). The reaction mixture was stirred at 70° C. (oil bath) for 14 h, cooled to room temperature, and diluted with ethyl acetate (100 mL) and hexanes (20 mL). The mixture was washed with water (4×20 mL) and brine (40 mL) and concentrated under reduced pressure. Flash silica gel column chromatography-using ethyl acetate/CH₂Cl₂ (1:25, 1:20, v/v) gave the desired product 31 as a thick yellow oil (718 mg, 83% yield). ¹H NMR (300 MHz, CDCl₃) δ 1.44 (s, 9H), 1.48-1.68 (m, 4H), 2.12-2.38 (m, 2H), 2.48-2.60 (m, 2H), 3.10-3.24 (m, 2H), 3.75 (s, 3H), 3.97-4.06 (m, 2H), 4.41-4.52 (m, 1H), 4.70 (br s, 1H), 5.09 (s, 2H), 5.25-5.37 (m, 1H), 6.70-6.80 (m, 2H), 6.95-7.09 (m, 2H), 7.30-7.38 (m, 5H). m/z (ESI) 515 [C₂₈H₃₈N₂O₇+H]⁺.

### {4-[4-(3-tert-Butoxycarbonylamino-3-carbamoylpropoxy)phenyl]butyl}carbamic acid benzyl ester (32)

Ammonia (7 M in methanol, 20 mL) was added to a solution of N-Boc methyl ester 31 (718 mg, 1.40 mmol) in methanol (5 mL) and the mixture was stirred at room temperature in a sealed tube for 40 h. The mixture was concentrated by rotary evaporation and purified by flash silica gel column chromatography using methanol/dichloromethane (1:30, 1:20, v/v) to give the desired amide 32 as a white solid (436 mg, 63% yield). ¹H NMR (300 MHz, CDCl₃) δ 1.45 (s, 9H), 1.48-1.68 (m, 4H), 2.09-2.32 (m, 2H), 2.48-2.61 (m, 2H), 3.08-3.25 (m, 2H), 3.97-4.20 (m, 2H), 4.30-4.45 (m, 1H), 4.75 (br s, 1H), 5.09 (s, 2H), 5.48-5.58 (m, 1H), 5.62 (br s, 1H), 6.38 (br s, 1H), 6.75-6.85 (m, 2H), 6.99-7.10 (m, 2H), 7.28-7.40 (m, 5H). m/z (ESI) 500 [C₂₇H₃₇N₃O₆+H]⁺.

### Example 13

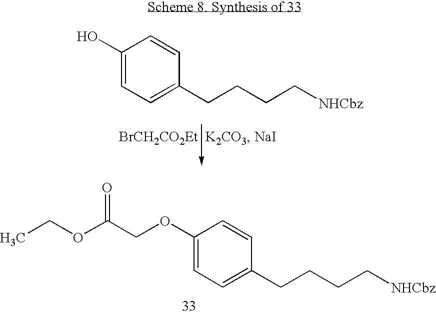

### Synthesis of [4-(4-Benzyloxycarbonylaminobutyl)phenoxy]acetic acid ethyl ester (33)

A solution of [4-(4-hydroxyphenyl)butyl]carbamic acid benzyl ester (2.0 g, 6.7 mmol), potassium carbonate (1.0 g, 7.3 mmol), sodium iodide (0.4 g, 2.7 mmol), and DMF (10 mL) was stirred for 30 minutes. A solution of ethyl bromoacetate (0.8 mL, 7.4 mmol) in DMF (10 mL) was added dropwise to the reaction. The reaction was further stirred at room temperature for 3 days, and then poured into water (200 mL). The product was extracted with ethyl acetate. The organic layer was dried over sodium sulfate and concentrated in vacuo. The residue was purified by flash chromatography (silica gel, 1:5 ethyl acetate/hexanes, v/v) to provide the desired product 33 (2.3 g, 89%) as a white solid. ¹H NMR (300 MHz, CDCl₃) δ 1.34 (t, 3H), 1.57 (m, 4H), 2.56 (t, 2H), 3.20 (q, 2H), 4.25 (q, 2H), 4.59 (s, 2H), 5.10 (s, 2H), 6.85 (d, 2H), 7.06 (d, 2H), 7.38 (m, 5H).

### Example 14

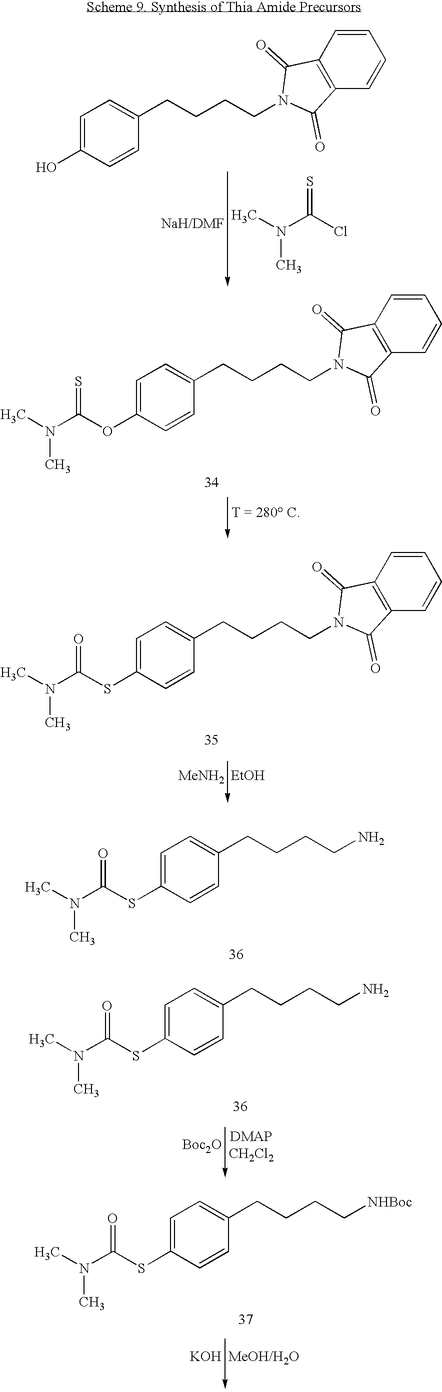

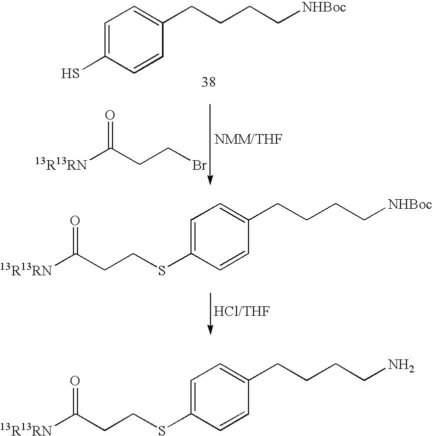

### Example 14

### N-{4-[4-(Dimethylthiocarbamoyloxy)phenyl]butyl}phthalimide (34)

A suspension of sodium hydride in mineral oil (0.44 g of 60%) in anhydrous DMF (10 mL) was cooled to 0° C. N-[4-(4-hydroxyphenyl)butyl]phthalimide (2.95 g, 10 mmol) dissolved in dry DMF (15 ml) was added into the mixture which was then stirred for 30 min at 0° C. and allowed to warm to room temperature over 1 h. To the mixture was added portionwise N,N-dimethylthiocarbamoyl chloride (1.35 g, 11 mmol) dissolved in DMF (10 ml). The newly formed mixture was stirred at room temperature overnight, then at 50° C. for 1 h, and was cooled back to room temperature, at which point methanol (10 mL) was added into the mixture. The solvent was removed under reduced pressure. The residue was purified by flash chromatography over silica gel (dichloromethane/hexane/ethyl acetate, 10:1:0.2, v/v) to give N-{4-[4-(dimethythiocarbamoyloxy)phenyl]butyl}phthalimide 34 (2.27 g, 59%) as a slightly yellow solid. ¹H NMR (300 MHz, CDCl₃) δ 1.72 (m, 4H), 2.66 (m, 2H), 3.33 (s, 3H), 3.45 (s, 3H), 3.70 (m, 2H), 6.96 (d, 2H), 7.18 (d, 2H), 7.71 (m, 2H), 7.84 (m, 2H). m/z (ESI) 383 [C₂₁H₂₂N₂O₃S+H]⁺.

### N-{4-[4-(Dimethylcarbamoylthio)phenyl]butyl}phthalimide (35)

### N-{4-[4-(Dimethythiocarbamoyloxy)phenyl]butyl}phthalimide 34

(2.1 g, 5.4 mmol) was placed in preheated sand bath at 230° C. The temperature was raised to 280° C. and the melted compound was kept at this temperature for 2 h in argon atmosphere. The mixture was cooled and the residue was purified by flash chromatography over silica gel (dichloromethane/hexane/ethyl acetate, 10:1:0.2, v/v) to give 35 (1.2 g, 57%) as a white powder. ¹H NMR (300 MHz, CDCl₃) δ 1.70 (m, 4H), 2.66 (t, 2H), 3.05 (br s, 3H), 3.71 (t, 2H), 6.96 (d, 2H), 7.19 (d, 2H), 7.38 (d, 2H), 7.70 (m, 2H), 7.83 (m, 2H). m/z (ESI) 383 [C₂₁H₂₂N₂O₃S+H]⁺.

### {4-[4-(Dimethylcarbamoylthio)phenyl]butylamine (36)

Phthalimide 35 (1.1 g, 2.8 mmol) was dissolved in a solution containing 6.6% methylamine in ethanol (100 mL) and allowed to stir at room temperature overnight. The solvent was removed under reduced pressure and the residue was purified by flash chromatography (silica gel, chloroform/methanol/ammonium hydroxide, 10:1:0.1, v/v) to afford the free amine 36 (0.31 g, 42%) as a white powder. ¹H NMR (300 MHz, CD₃OD) δ 1.51 (m, 2H), 1.65 (m, 2H), 2.66 (m, 4H), 3.02 (m, 6H), 7.23 (d, 2H), 7.35 (d, 2H).

### N-tert-Butoxycarbonyl-{4-[4-(dimethylcarbamoylthio)phenyl]butylamine (37)

Di-tert-butyldicarbonate (0.35 g, 1.6 mmol) was added to a solution of 36 (0.31 g, 1.22 mmol), 4-Dimethylaminopyridine (DMAP), and dichloromethane (50 mL). The mixture was stirred at room temperature for 26 h. The solvent was removed under reduced pressure and the residue was purified by flash chromatography over silica gel (hexane/ethyl acetate, 3:1, v/v) to give 36 (0.36 g, 83%) as a white powder. ¹H NMR (300 MHz, CDCl₃) δ 1.44 (s, 9H), 1.51 (m, 2H) 1.64 (m, 2H), 3.08 (m, 8H), 4.48 (br s, 1H), 7.18 (d, 2H), 7.39 (d, 2H).

### N-[4-(4-Mercaptophenyl)butyl]carbamic acid tert-butyl ester (38)

N-tert-Butoxycarbonyl-{4-[4-(dimethylcarbamoylthio)phenyl]butylamine 37 (0.35 g, 1.02 mmol) was dissolved in MeOH (8 mL). KOH (0.19 g, 3.4 mmol) dissolved in water (2 ml) was added. The mixture was stirred under reflux for 6 h and cooled to room temperature. The solvent was removed under reduced pressure. The residue was dissolved in water and acidified with 5% aqueous HCl to pH 5. The solvent was removed again under reduced pressure and the residue was purified by flash chromatography over silica gel (hexane/ethyl acetate, 3:1, v/v) to give the desired thiophenol 38 (0.13 g, 45%) as a clear oil. ¹H NMR (300 MHz, CDCl₃) δ 1.43 (s, 9H), 1.49 (m, 2H) 1.59 (m, 2H), 2.57 (t, 2H), 3.12 (m, 2H), 3.38 (s, 1H), 4.47 (br s, 1H), 7.04 (d, 2H), 7.19 (d, 2H).

### Example 14

### 4-(4-Carboxymethylphenyl)butylamine (41). Methanesulfonic acid 4-(4-carboxymethylphenyl)butyl ester (39)

Compound 39 was prepared according to the published procedure¹. ¹H NMR (300 MHz, CDCl₃) δ 1.75 (m, 4H), 2.78 (m, 2H), 3.12 (s, 3H), 3.88 (s, 3H), 4.22 (m, 2H), 7.28 (d, 2H), 7.98 (d, 2H).

### 4-(4-Carboxymethylphenyl)butylazide (7). Typical procedure C

Compound 39 (6 g, 0.02 mol) was dissolved in 80 ml of dry DMF then sodium azide (1.8 g, 0.027 mol) was added. The suspension was stirred at 80° C. (oil bath) for 3 h. The solvent was then removed at reduced pressure and the residual oil was treated with CH₂Cl₂ (100 mL). The resulting solution was washed with water (2×100 mL), brine and dried over magnesium sulfate. The solvent was removed under reduced pressure then the residue was redissolved in a 1:1 mixture of ethyl acetate/hexanes (200 mL) and passed through a pad of silica gel. The solvent was removed under reduced pressure to give 4.1 g (85%) of 7 as clear oil. ¹H NMR (300 MHz, CDCl₃) δ 1.68 (m, 4H), 2.22 (t, 2H), 3.29 (t, 3H), 3.92 (s, 3H), 7.28 (d, 2H), 7.98 (d, 2H).

### 4-(4-Carboxymethylphenyl)butylamine (41)

Azide 40 (1.7 g, 7.2 mmol) and triphenylphosphine (1.9 g, 7.2 mmol) were dissolved in a 10% solution of water in THF (66 mL) and stirred overnight at 25° C. Then more triphenylphosphine (0.8 g, 3 mmol) was added and the heating was continued at 60° C. (oil bath) for 6 h. The solvent was removed under reduced pressure and the residue was treated with 2M HCl (100 mL) and extracted with ethyl acetate (2×50 mL). The water fraction was collected and ammonium hydroxide was added until the pH reached approximately 13. The mixture was extracted with ethyl acetate (2×100 mL) then the organic fraction was washed with brine, water and dried with sodium sulfate. Ethyl acetate was removed under reduced pressure to give 0.8 g (53%) of amine 41.

### Example 15

### Synthesis of [4-(4-Aminobutyl)phenoxy]acetic acid ethyl ester (43)

### [4-(4-Benzyloxycarbonylaminobutyl)phenoxy]acetic acid ethyl ester (42)

Sodium hydride (60% dispersion in mineral oil) (0.24 g, 10.05 mmol) was added to a cold (0° C.) solution of 4-(4-hydroxyphenyl)butylamine (2 g, 6.68 mmol) in THF (150 mL) under nitrogen atmosphere. The reaction mixture was allowed to warm up to room temperature over 0.5 h with stirring, then ethyl bromoacetate (0.96 mL, 8.02 mmol) and tetrabutylammonium iodide (0.25 g, 0.67 mmol) was sequentially added. The reaction was further stirred at room temperature overnight. Silica gel (25 mL) was added into the mixture and the solvent was evaporated. The impregnated silica gel was subjected to column chromatography purification (silica gel, 5:1 hexanes/ethyl acetate). 2.42 g (94%) of 42 was obtained as a white solid. ¹H NMR (300 MHz, CDCl₃) δ 1.30 (t, 3H), 1.57 (m, 4H), 2.58 (m, 2H), 3.20 (m, 2H), 4.28 (m, 2H), 4.58 (s, 2H), 4.74 (br s, 1H), 5.10 (br s, 2H), 6.82 (m, 2H), 7.08 (m, 2H), 7.38 (br s, 5H).

### [4-(4-Aminobutyl)phenoxy]acetic acid ethyl ester (43)

A suspension of 42 (1.11 g, 2.88 mmol) and 10% palladium on carbon (0.40 g, wet) in methanol (50 mL) was stirred at room temperature for 2 h under atmospheric pressure of hydrogen. The mixture was then filtered through a silica gel pad. The solvent was evaporated to provide 43 (0.64 g, 88%) as a white solid. ¹H NMR (300 MHz, DMSO-d₆) δ 1.21 (m, 3H), 1.30-1.63 (m, 6H), 3.13 (m, 2H), 4.17 (m, 2H), 4.72 (br s, 2H), 6.84 (m, 2H), 7.10 (m, 2H).

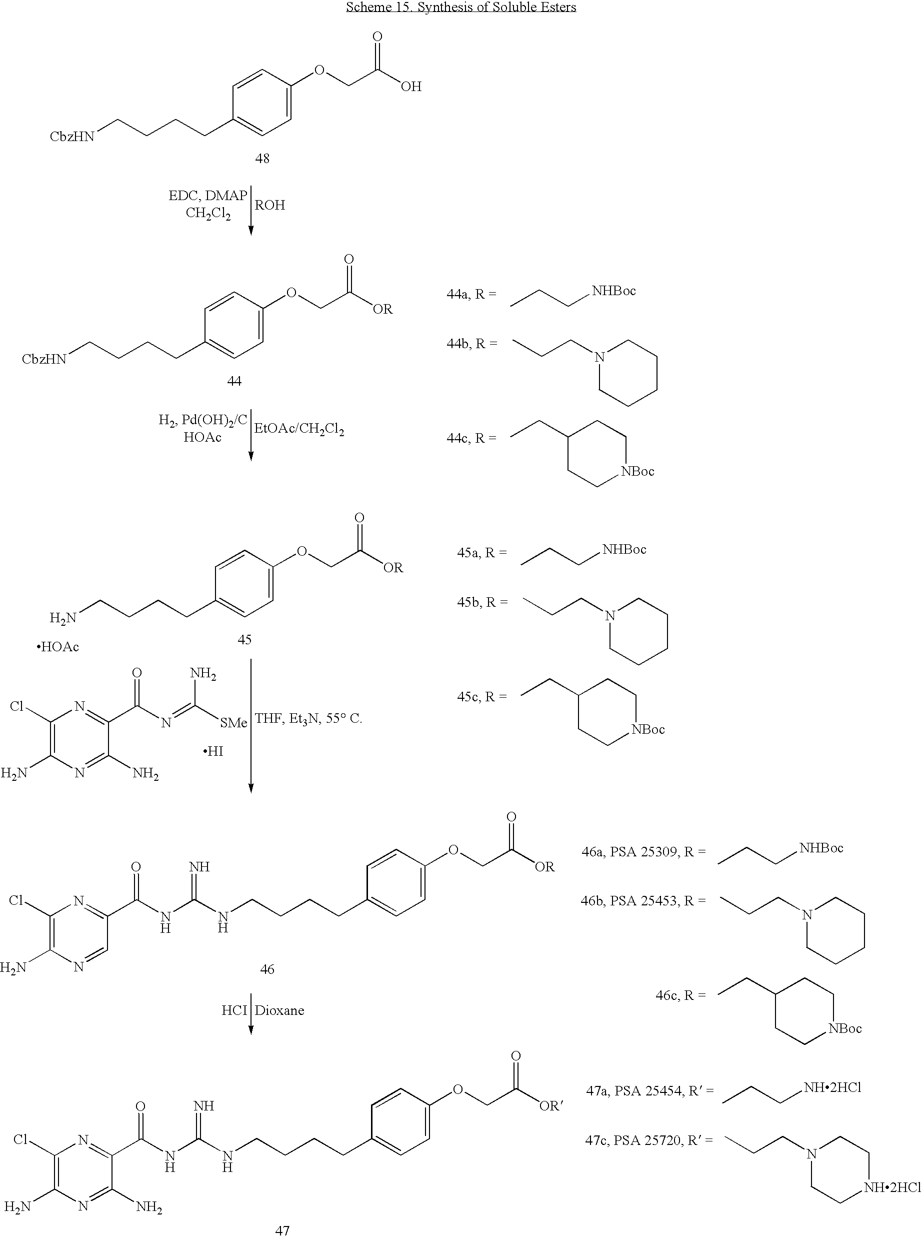

### Example 16

### Synthesis of (4-{4-[N′-(3,5-diamino-6-chloropyrazine-2-carbonyl)guanidino]butyl}-phenoxy)acetic acid 2-aminoethyl ester dihydrochloride (PSA 25454)

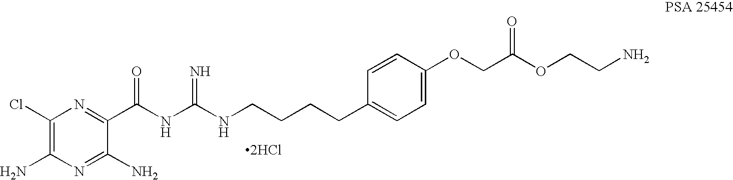

### [4-(4-Benzyloxycarbonylaminobutyl)phenoxy]acetic acid 2-tert-butoxycarbonylamino-ethyl ester (44a)

[4-(4-Benzyloxycarbonylaminobutyl)phenoxy]acetic acid 48 (240 mg, 0.67 mmol) was dissolved in anhydrous CH₂Cl₂ (6 mL). To the solution was added DMAP (98 mg, 0.80 mmol), followed by EDC-HCl (154 mg, 0.81 mmol). The reaction mixture was stirred at room temperature for 15 min. (2-Hydroxyethyl)carbamic acid tert-butyl ester (0.21 mL, 1.34 mmol) was then added and the stirring was continued for 16 h. Solvent was removed by rotary evaporation. The residue was subjected to flash silica gel column chromatography eluting with ethyl acetate/dichloromethane (1:20, 1:15, and 1:10, v/v) to give the desired ester 44a as a colorless oil (162 mg, 48% yield). ¹H NMR (300 MHz, CDCl₃) δ 1.35-1.65 (m, 4H), 1.44 (s, 9H), 2.45-2.58 (m, 2H), 3.10-3.22 (m, 2H), 3.25-3.42 (m, 2H), 4.18-4.28 (m, 2H), 4.62 (s, 2H), 4.70 (br s, 1H), 4.84 (br s, 1H), 5.08 (s, 2H), 6.81 (d, 2H), 7.07 (d, 2H), 7.25-7.38 (m, 5H). m/z (ESI) 501 [C₂₇H₃₆N₂O₇+H]⁺.

### [4-(4-Aminobutyl)phenoxy]acetic acid 2-tert-butoxycarbonylaminoethyl ester ethyl acetate (45a)

A solution of [4-(4-benzyloxycarbonylaminobutyl)phenoxy]acetic acid 2-tert-butoxycarbonylaminoethyl ester 44a (162 mg, 0.32 mmol) and acetic acid (3 drops) in ethyl acetate (3 mL) and dichloromethane (6 mL) was stirred at room temperature for 16 h under hydrogen atmosphere in the presence of palladium hydroxide on activated charcoal (80 mg, 60% wet). The catalyst was removed by suction filtration and the liquid filtrate was concentrated in vacuo to give [4-(4-aminobutyl)phenoxy]acetic acid 2-tert-butoxycarbonylaminoethyl ester ethyl acetate 45a as an off-white solid (100 mg, 84% yield) which was used directly without purification. m/z (ESI) 367 [C₁₉H₃₀N₂O₅+H]⁺.

### (4-{4-[N′-(3,5-Diamino-6-chloropyrazine-2-carbonyl)guanidino]butyl}phenoxy)acetic acid 2-tert-butoxycarbonylaminoethyl ester (46a, PSA 25309)

A solution of [4-(4-aminobutyl)phenoxy]acetic acid 2-tert-butoxycarbonylaminoethyl ester ethyl acetate 45a (50 mg, 0.27 mmol) and triethylamine (0.27 mL, 1.53 mmol) in anhydrous THF (4 mL) was stirred at 55° C. (oil bath) for 15 min. To the solution was added 1-(3,5-diamino-6-chloropyrazine-2-carbonyl)-2-methylisothiourea hydriodide (130 mg, 0.34 mmol) in two portions during a period of 15 min. The reaction mixture was stirred at 55° C. for 1 h and cooled to room temperature. The mixture was concentrated by rotary evaporation. The crude residue was purified by flash silica gel column chromatography eluting with dichloromethane/methanol/concentrated ammonium hydroxide (200:10:0, 200:10:1, and 150:10:1, v/v) to give (4-{4-[N-(3,5-diamino-6-chloropyrazine-2-carbonyl)guanidino]butyl}phenoxy)acetic acid 2-tert-butoxycarbonylaminoethyl ester 46a (PSA 25309) as a yellow solid (60 mg, 76% yield). An analytical pure sample was obtained by semi-preparative HPLC purification (acetonitrile/water). mp 80-82° C. ¹H NMR (300 MHz, CD₃OD) δ 1.45 (s, 9H), 1.60-1.78 (m, 4H), 2.56-2.66 (m, 2H), 3.30-3.38 (m, 4H), 4.15-4.21 (m, 2H), 4.64 (s, 2H), 6.81 (d, 2H), 7.10 (d, 2H). m/z (ESI) 579 [C₂₅H₃₅ClN₈O₆+H]⁺.

### (4-{4-[N′-(3,5-Diamino-6-chloropyrazine-2-carbonyl)guanidino]butyl}phenoxy)acetic acid 2-aminoethyl ester dihydrochloride (47a, PSA 25454)

(4-{4-[N′-(3,5-Diamino-6-chloropyrazine-2-carbonyl)guanidino]butyl}phenoxy)acetic acid 2-tert-butoxycarbonylaminoethyl ester 46a (24 mg, 0.041 mmol) was treated with HCl (4 N in dioxane, 1 mL, 4 mmol) at room temperature for 14 h. The reaction mixture was concentrated in vacuo and the residue was purified by semi-preparative HPLC method (acetonitrile/water) to afford the desired product 47a (PSA 25454, 7 mg, 31%) as a yellow solid. mp 66-68° C. ¹H NMR (500 MHz, CD₃OD) δ 1.65-1.76 (m, 4H), 2.60-2.68 (m, 2H), 3.25-3.31 (m, 2H), 3.33-3.39 (m, 2H), 4.40-4.45 (m, 2H), 4.75 (s, 2H), 6.86 (d, 2H), 7.11 (d, 2H). m/z (APCI) 479 [C₂₀H₂₇ClN₈O₄+H]⁺.

### Example 17

### Synthesis of (4-{4-[N′-(3,5-diamino-6-chloropyrazine-2-carbonyl)guanidino]butyl}-phenoxy)acetic acid 2-piperidin-1-yl-ethyl ester (PSA 25453)

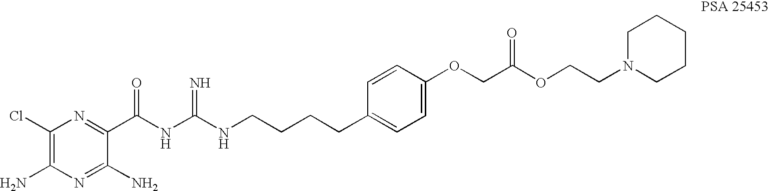

### [4-(4-Benzyloxycarbonylaminobutyl)phenoxy]acetic acid 2-piperidin-1-yl-ethyl ester (44b)

Following the same procedure for the synthesis of compound 44a, compound 49b was synthesized from compound 48 in 60% yield as a colorless oil. ¹H NMR (300 MHz, CDCl₃) δ 1.33-1.68 (m, 1H), 2.30-2.45 (m, 4H), 2.50-2.65 (m, 4H), 3.10-3.25 (m, 2H), 4.28-4.35 (m, 2H), 4.60 (s, 2H), 4.75 (br s, 1H), 5.08 (s, 2H), 6.80 (d, 2H), 7.05 (d, 2H), 7.28-7.38 (m, 5H). m/z (ESI) 469 [C₂₇H₃₆N₂O₅+H]⁺.

### [4-(4-Aminobutyl)phenoxy]acetic acid 2-piperidin-1-yl-ethyl ester ethyl acetate (45b)

Following the same procedure for the synthesis of compound 45a, compound 45b was synthesized from compound 44b in 80% yield as an off-white solid, and used directly without purification. m/z (ESI) 335 [C₁₉H₃₀N₂O₃+H]⁺.

### (4-{4-[N′-(3,5-Diamino-6-chloropyrazine-2-carbonyl)guanidino]butyl}phenoxy)acetic acid 2-piperidin-1-yl-ethyl ester (46b, PSA 25453)

Following the same procedure for the synthesis of compound 46a, compound 46b was synthesized from compound 45b. One fifth of the crude reaction mixture, after concentration under vacuum, was purified by semi-preparative HPLC method (acetonitrile/water) to afford pure 46b (PSA 25453) as a yellow solid. mp 60-62° C.

¹H NMR (500 MHz, DMSO-d₆) δ 1.30-1.80 (m, 10H), 2.50-2.60 (m, 2H), 2.88-2.98 (m, 2H), 3.25-3.35 (m, 2H), 3.35-3.48 (m, 4H), 4.42-4.48 (m, 2H), 4.78 (s, 2H), 6.88 (d, 2H), 7.12 (d, 2H), 7.42 (s, 2H), 8.75 (s, 1H), 8.90 (s, 1H), 9.15 (s, 1H), 9.65 (s, 1H), 10.45 (s, 1H). m/z (ESI) 547 [C₂₅H₃₅ClN₈O₄+H]⁺.

### Example 18

### Synthesis of (4-{4-[N′-(3,5-diamino-6-chloropyrazine-2-carbonyl)guanidino]butyl}-phenoxy)acetic acid piperidin-4-yl-methyl ester dihydrochloride (PSA 25720)

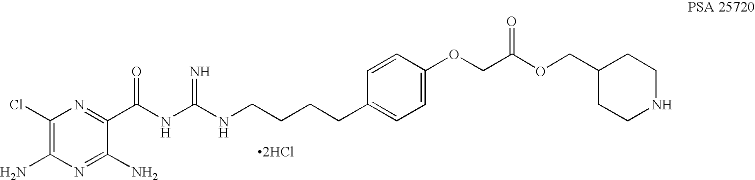

### 4-{2-[4-(4-Benzyloxycarbonylaminobutyl)phenoxy]acetoxymethyl}piperidine-1-carboxylic acid tert-butyl ester (44c)

Following the same procedure for the synthesis of compound 44a, compound 44c (a colorless oil) was synthesized in 71% yield by the reaction of compound 48 with 4-hydroxymethylpiperidine-1-carboxylic acid tert-butyl ester, synthesis of which is described below. ¹H NMR (300 MHz, CDCl₃) δ 1.05-1.20 (m, 2H), 1.45 (s, 9H), 1.50-1.65 (m, 6H), 1.70-1.88 (m, 1H), 2.50-2.75 (m, 4H), 3.12-3.26 (m, 2H), 4.00-4.15 (m, 4H), 4.60 (s, 2H), 4.88 (br s, 1H), 5.08 (s, 2H), 6.80 (d, 2H), 7.08 (d, 2H), 7.30-7.48 (m, 5H). m/z (ESI) 555 [C₃₁H₄₂N₂O₇+H]⁺.

Synthesis of 4-hydroxymethylpiperidine-1-carboxylic acid tert-butyl ester:

To a solution of piperidin-4-yl-methanol (2.59 g, 22.5 mmol) in 1,4-dioxane (60 mL) was added aqueous sodium hydroxide (1 N, 35 mL). The mixture was chilled in an ice bath. To the chilled mixture was added Boc₂O (7.36 g, 33.7 mmol) in one portion. The reaction mixture was stirred at 0° C. for 30 min and at room temperature for 16 h, and concentrated under vacuum to about one-third of its original volume. The remaining mixture was diluted with water and dichloromethane. Layers were separated and the aqueous layer was further extracted with dichloromethane. The combined organics were concentrated under vacuum and purified by flash silica gel column chromatography eluting with ethyl acetate/hexanes (1:10, 1:4, and 1:1, v/v) to give 4-hydroxymethylpiperidine-1-carboxylic acid tert-butyl ester (4.71 g, 97% yield) as a white solid. ¹H NMR (300 MHz, CDCl₃) δ 1.10-1.20 (m, 2H), 1.30 (t, 1H), 1.47 (s, 9H), 1.60-1.69 (m, 1H), 1.69-1.75 (m, 2H), 2.65-2.75 (m, 2H), 3.48-3.52 (m, 2H), 4.05-4.18 (m, 2H). m/z (ESI) 216 [C₁₁H₂₁NO₃+H]⁺.

### 4-{2-[4-(4-Aminobutyl)phenoxy]acetoxymethyl}piperidine-1-carboxylic acid tert-butyl ester ethyl acetate (45c)

Following the same procedure for the synthesis of compound 45a, compound 45c was synthesized from compound 44c in 99% yield as an off-white solid, and used directly without purification. m/z (ESI) 421 [C₂₃H₃₆N₂O₅+H]⁺.

### 4-[2-(4-{4-[N′-(3,5-Diamino-6-chloropyrazine-2-carbonyl)guanidino]butyl}phenoxy)-acetoxymethyl]piperidine-1-carboxylic acid tert-butyl ester (46c)

Following the same procedure for the synthesis of compound 46a, compound 46c was synthesized from compound 45c. One-third of the crude reaction mixture, after concentration under vacuum, was purified by semi-preparative HPLC method (acetonitrile/water) to give 46c as a yellow solid. m/z (ESI) 633 [C₂₉H₄₁ClN₈O₆+H]⁺.

### (4-{4-[N′-(3,5-Diamino-6-chloropyrazine-2-carbonyl)guanidino]butyl}phenoxy)acetic acid piperidin-4-yl-methyl ester dihydrochloride (47c, PSA 25720)

Following the same procedure for the synthesis of compound 47a, compound 47c (PSA 25720) was synthesized from compound 46c in 39% yield as a yellow solid. mp 100-102° C. ¹H NMR (500 MHz, DMSO-d₆) δ 1.30-1.45 (m, 2H), 1.50-1.65 (m, 4H), 1.72-1.80 (m, 2H), 1.88-1.98 (m, 1H), 2.52-2.60 (m, 2H), 2.78-2.88 (m, 2H), 3.20-3.29 (m, 2H), 4.00 (d, 2H), 4.75 (s, 2H), 6.85 (d, 2H), 7.12 (d, 2H), 7.40 (br s, 2H), 8.80 (br s, 3H), 9.20 (br s, 2H). m/z (APCI) 533 [C₂₄H₃₃ClN₈O₄+H]⁺.

While the invention has been described with reference to preferred aspects, it is to be understood that variations and modifications may be resorted to as will be apparent to those skilled in the art. Such variations and modifications are to be considered within the purview and the scope of the claims appended hereto.

What is claimed is:

1. A prophylactic treatment method comprising:
administering a prophylactically effective amount of a sodium channel blocker according to Formula (I) or a pharmaceutically acceptable salt thereof to an individual in need of prophylactic treatment against infection or disease from one or more airborne pathogens, wherein Formula (I) is 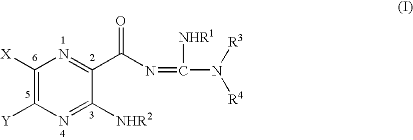
where X is hydrogen, halogen, trifluoromethyl, lower alkyl, unsubstituted or substituted phenyl, lower alkyl-thio, phenyl-lower alkyl-thio, lower alkyl-sulfonyl, or phenyl-lower alkyl-sulfonyl; Y is hydrogen, hydroxyl, mercapto, lower alkoxy, lower alkyl-thio, halogen, lower alkyl, unsubstituted or substituted mononuclear aryl, or —N(R²)₂; R¹ is hydrogen or lower alkyl; each R² is, independently, —R⁷, —(CH₂)ₘ—OR⁸, —(CH₂)ₘ—NR⁷R¹⁰, —(CH₂)ₙ(CHOR⁸)(CHOR⁸)ₙ—CH₂OR⁸, —(CH₂CH₂O)ₘ—R⁸, —(CH₂CH₂O)ₘ—CH₂CH₂NR⁷R¹⁰, —(CH₂)ₙ—C(═O)NR⁷R¹⁰, —(CH₂)ₙ-Z_{g}-R⁷, —(CH₂)ₘ—NR¹⁰—CH₂(CHOR⁸)(CHOR⁸)ₙ—CH₂OR⁸, —(CH₂)ₙ—CO₂R⁷, or 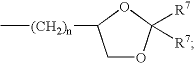
wherein when two —CH₂OR⁸ groups are located 1,2- or 1,3- with respect to each other the R⁸ groups may be joined to form a cyclic mono- or di-substituted 1,3-dioxane or 1,3-dioxolane;
R³ and R⁴ are each, independently, hydrogen, a group represented by formula (A), lower alkyl, hydroxy lower alkyl, phenyl, phenyl-lower alkyl, (halophenyl)-lower alkyl, lower-(alkylphenylalkyl), lower (alkoxyphenyl)-lower alkyl, naphthyl-lower alkyl, or pyridyl-lower alkyl, with the proviso that at least one of R³ and R⁴ is a group represented by formula (A): 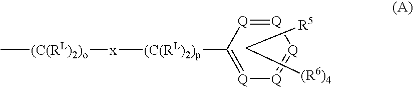
wherein
each R^{L} is, independently, —R⁷, —(CH₂)ₙ—OR⁸, —O—(CH₂)ₘ—OR⁸, —(CH₂)—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose, 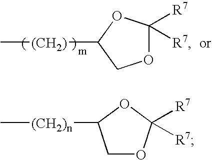
wherein when two —CH₂OR⁸ groups are located 1,2- or 1,3- with respect to each other the R⁸ groups may be joined to form a cyclic mono- or di-substituted 1,3-dioxane or 1,3-dioxolane;
each o is, independently, an integer from 0 to 10;
each p is an integer from 0 to 10;
with the proviso that the sum of o and p in each contiguous chain is from 1 to 10;
each x is, independently, O, NR¹⁰, C(═O), CHOH, C(═N—R¹⁰), CHNR⁷R¹⁰, or represents a single bond;
each R⁵ is independently, —(CH₂)ₙ—CO₂R¹³, Het-(CH₂)ₘ—CO₂R¹³, —(CH₂)ₙ-Z_{g}-CO₂R¹³, Het-(CH₂)ₘ-Z_{g}-CO₂R¹³, —(CH₂)ₙ—NR¹⁰—(CH₂)ₘ(CHOR⁸)ₙ—CO₂R¹³, Het-(CH₂)ₘ—NR¹⁰—(CH₂)ₘ(CHOR⁸)ₙ—CO₂R¹³, —(CH₂)ₙ—(CHOR⁸)ₘ—CO₂R¹³, Het-(CH₂)ₘ—(CHOR⁸)ₘ—CO₂R¹³, —(CH₂)ₙ—(CHOR⁸)ₘ-Z_{g}-CO₂R¹³, Het-(CH₂)ₙ—(CHOR⁸)ₘ-Z_{g}-CO₂R¹³, —(CH₂)ₙ-Z_{g}-(CH₂)ₘ—CO₂R¹³, —(CH₂)ₙ-Z_{g}-(CH₂)ₘ—CO₂R¹³, —(CH₂)ₙ-Z_{g}(CHOR⁸)ₘ-Z_{g}-CO₂R¹³, Het-(CH₂)ₙ-Z_{g}-(CHOR⁸)ₘ-Z_{g}-CO₂R¹³, —(CH₂)ₙ—CONH—C(═NR¹³)—NR¹³R¹³, Het-(CH₂)ₙ—CO—NH—C(═NR¹³)—NR¹³R¹³, —(CH₂)ₙ-Z_{g}-CONH—C(═NR¹³)—NR¹³R¹³, Het-(CH₂)ₙ-Z_{g}-CONH—C(═NR¹³)—NR¹³R¹³, —(CH₂)ₙ—NR¹⁰—(CH₂)ₘ(CHOR⁸)ₙ—CONH—C(═NR¹³)—NR¹³R¹³, Het-(CH₂)ₙ—NR¹⁰—(CH₂)ₘ(CHOR⁸)ₙ—CONH—C(═NR¹³)—NR¹³R¹³, —(CH₂)ₙ—(CHOR⁸)ₘ—CONH—C(═NR¹³)—NR¹³R¹³, Het-(CH₂)ₙ—(CHOR⁸)ₘ—CONH—C(═NR¹³)—NR¹³R¹³, —(CH₂)ₙ—(CHOR⁸)ₘ-Z_{g}-CONH—C(═NR¹³)—NR¹³R¹³, Het-(CH₂)ₙ—(CHOR⁸)ₘ-Z_{g}-CONH—C(═NR¹³)—NR¹³R¹³, —(CH₂)ₙ-Z_{g}-(CH₂)ₘCONH—C(═NR¹³)—NR¹³R¹³, Het-(CH₂)ₙ-Z_{g}-(CH₂)ₘCONH—C(═NR¹³)—NR¹³R¹³, —(CH₂)ₙ-Z_{g}-(CHOR⁸)ₘ-Z_{g}-CONH—C(═NR¹³)—NR¹³R¹³, Het-(CH₂)ₙ-Z_{g}-(CHOR⁸)ₘ-Z_{g}-CONH—C(═NR¹³)—NR¹³R¹³, —(CH₂)ₙ—CONR⁷—CONR¹³R¹³, Het-(CH₂)ₙ—CONR⁷—CONR¹³R¹³, —(CH₂)ₙ-Z_{g}-CONR⁷—CONR¹³R¹³, —(CH₂)ₙ-Z_{g}-CONR⁷—CONR¹³R¹³, —(CH₂)ₙ—NR¹⁰—(CH₂)ₘ(CHOR⁸)ₙ—CONR⁷—CONR¹³R¹³, Het-(CH₂)ₙ—NR¹⁰—(CH₂)ₘ(CHOR⁸)ₙ—CONR⁷—CONR¹³R¹³, —(CH₂)ₙ—(CHOR⁸)ₘ—CONR⁷—CONR¹³R¹³, Het-(CH₂)ₙ—(CHOR⁸)ₘ—CONR⁷—CONR¹³R¹³, —(CH₂)ₙ(CHOR⁸)ₘ-Z_{g} CONR⁷—CONR¹³R¹³, Het-(CH₂)ₙ—(CHOR⁸)ₘ-Z_{g}-CNR⁷—CONR¹³R¹³, —(CH₂)ₙ-Z_{g}-(CH₂)ₘCONR⁷—CONR¹³R¹³, Het-(CH₂)ₙ-Z_{g}-(CH₂)ₘCONR⁷—CONR¹³R¹³, —(CH₂)ₙ-Z_{g}(CHOR⁸)ₘ-Z_{g}-CONR⁷—CONR¹³R¹³, Het-(CH₂)ₙ-Z_{g}(CHOR⁸)ₘ-Z_{g}-CONR⁷—CONR¹³R¹³, —(CH₂)ₙ—CONR⁷SO₂NR¹³R¹³, Het-(CH₂)ₘ—CONR⁷SO₂NR¹³R¹³, —(CH₂)ₙ-Z_{g}-CONR⁷SO₂NR¹³R¹³, Het-(CH₂)ₘ-Z_{g}-CONR⁷SO₂NR¹³R¹³, —(CH₂)ₙ—NR¹⁰—(CH₂)ₘ(CHOR⁸)ₙ—CONR⁷SO₂NR¹³R¹³, Het-(CH₂)ₘ—NR¹⁰—(CH₂)ₘ—(CHOR⁸)ₙ—CONR⁷SO₂NR¹³R¹³, —(CH₂)ₙ—(CHOR⁸)ₘ—CONR⁷SO₂NR¹³R¹³, Het-(CH₂)ₘ—(CHOR⁸)ₘ—CONR⁷SO₂NR¹³R¹³, —(CH₂)ₙ—(CHOR⁸)ₘ-Z_{g}-CONR⁷SO₂NR¹³R¹³, Het-(CH₂)ₙ—(CHOR⁸)ₘ-Z_{g}-CONR⁷SO₂NR¹³R¹³, —(CH₂)ₙ-Z_{g}-(CH₂)ₘCONR⁷ SO₂NR¹³R¹³, Het-(CH₂)ₙ-Z_{g}-(CH₂)ₘCONR⁷SO₂NR¹³R¹³, —(CH₂)ₙ-Z_{g}-(CHOR⁸)ₘ-Z_{g}-CONR⁷SO₂NR¹³R¹³, Het-(CH₂)ₙ-Z_{g}-(CHOR⁸)ₘ-Z_{g}-CONR¹⁷SO₂NR¹³R¹³, —(CH₂)ₙ—SO₂NR¹³R¹³, Het-(CH₂)ₘ—SO₂NR¹³R¹³, —(CH₂)ₙ-Z_{g}-SO₂NR¹³R¹³, Het-(CH₂)ₘ-Z_{g}-SO₂NR¹³R¹³, —(CH₂)ₙ—NR¹⁰—(CH₂)ₘ(CHOR⁸)ₙ—SO₂NR¹³R¹³, Het-(CH₂)ₘ—NR¹⁰—(CH₂)ₘ(CHOR⁸)ₙ—SO₂NR¹³R¹³, —(CH₂)ₙ—(CHOR⁸)ₘ—SO₂NR¹³R¹³, Het-(CH₂)ₘ—(CHOR⁸)ₘ—SO₂NR¹³R¹³, —(CH₂)ₙ—(CHOR⁸)ₘ-Z_{g}-SO₂NR¹³R¹³, Het-(CH₂)ₙ—(CHOR⁸)ₘ-Z_{g}-SO₂NR¹³R¹³, —(CH₂)ₙ-Z_{g}-(CH₂)ₘSO₂NR¹³R¹³, Het-(CH₂)ₙ-Z_{g}-(CH₂)ₘSO₂NR¹³R¹³, —(CH₂)ₙ-Z_{g}-(CHOR⁸)ₘ-Z_{g}-SO₂NR¹³R¹³, Het-(CH₂)ₙ-Z_{g}-(CHOR⁸)ₘ-Z_{g}-SO₂NR¹³R¹³, —(CH₂)R¹³, —CONR¹³R¹³, Het-(CH₂)ₘ—CONR¹³R¹³, —(CH₂)ₙ-Z_{g}-CONR¹³R¹³, Het-(CH₂)ₘ-Z_{g}-CONR¹³R¹³, —(CH₂)ₙ—NR¹⁰—(CH₂)ₘ(CHOR⁸)ₙ—CONR¹³R¹³, Het-(CH₂)ₘ—NR¹⁰—(CH₂)ₘ(CHOR⁸)ₙ—CONR¹³R¹³, —(CH₂)ₙ—(CHOR⁸)ₘ—CONR¹³R¹³, Het-(CH₂)ₘ—(CHOR⁸)ₘ—CONR¹³R¹³, —(CH₂)ₙ—(CHOR⁸)ₘ-Z_{g}-CONR¹³R¹³, Het-(CH₂)ₙ—(CHOR⁸)ₘ-Z_{g}-CONR¹³R¹³, —(CH₂)ₙ-Z_{g}-(CH₂)ₘCONR¹³R¹³, Het-(CH₂)ₙ-Z_{g}-(CH₂)ₙ-Z_{g}-(CHOR⁸)ₘ-Z_{g}-CONR¹³R¹³, —(CH₂)ₙ—CONR⁷COR¹³, Het-(CH₂)ₘ—CONR⁷COR¹³, —(CH₂)ₙ-Z_{g}-CONR⁷COR¹³, Het-(CH₂)ₘ-Z_{g}-CONR⁷COR¹³, —(CH₂)ₙ—NR¹⁰—(CH₂)ₘ(CHOR⁸)ₙ—CONR⁷COR¹³, Het-(CH₂)ₘ—NR¹⁰—(CH₂)ₘ(CHOR⁸)ₙ—CONR⁷COR¹³, —(CH₂)ₙ—(CHOR⁸)ₘ—CONR⁷COR¹³, Het-(CH₂)ₘ—(CHOR⁸)ₘ—CONR⁷COR¹³, —(CH₂)ₙ—(CHOR⁸)ₘ-Z_{g}-CONR⁷COR¹³, Het-(CH₂)ₙ—(CHOR⁸)ₘ-Z_{g}-CONR⁷COR¹³, —(CH₂)ₙ-Z_{g}-(CH₂)ₘCONR⁷COR¹³, —(CH₂)ₙ-Z_{g}-(CH₂)ₘCONR⁷COR¹³, Het-(CH₂)ₙ-Z_{g}-(CHOR⁸)ₘ-Z_{g}-CONR⁷COR¹³, —(CH₂)ₙ—CONR⁷CO₂R¹³, —(CH₂)ₙ-Z_{g}-CONR⁷CO₂R¹³, Het-(CH₂)ₘ-Z_{g}-CONR⁷CO₂R¹³, —(CH₂)ₙ—NR¹⁰—(CH₂)ₘ(CHOR⁸)ₙ—CONR⁷CO₂R¹³, Het-(CH₂)ₘ—NR¹⁰—(CH₂)ₘ(CHOR⁸)ₙ—CONR⁷CO₂R¹³, —(CH₂)ₙ—(CHOR⁸)ₘ—CONR⁷CO₂R¹³, Het-(CH₂)ₘ—(CHOR⁸)ₘ—CONR⁷CO₂R¹³, —(CH₂)ₙ—(CHOR⁸)ₘ-Z_{g}-CONR⁷CO₂R¹³, Het-(CH₂)ₙ—(CHOR⁸)ₘ-Z_{g}-CONR⁷CO₂R¹³, —(CH₂)ₙ-Z_{g}-(CH₂)ₘCONR⁷CO₂R¹³, Het-(CH₂)ₙ-Z_{g}-(CH₂)ₘCONR⁷CO₂R¹³, —(CH₂)ₙ-Z_{g}-(CHOR⁸)ₘ-Z_{g}-CONR⁷CO₂R¹³, Het-(CH₂)ₙ-Z_{g}-(CHOR⁸)ₘ-Z_{g}-CONR⁷CO₂R¹³, —(CH₂)ₙ—NH—C(═NR¹³)—NR¹³R¹³, Het-(CH₂)ₘ—NH—C(═NR¹³)—NR¹³R¹³, —(CH₂)ₙ-Z_{g}-NH—C(═NR¹³)—NR¹³R¹³, Het-(CH₂)ₘ-Z_{g}-NH—C(═NR¹³)—NR¹³R¹³, —(CH₂)ₙ—NR¹⁰—(CH₂)ₘ(CHOR⁸)ₙ—NH—C(═NR¹³)—NR¹³R¹³, Het-(CH₂)ₘ—NR¹⁰—(CH₂)ₘ(CHOR⁸)ₙ—NH—C(═NR¹³)—NR¹³R¹³, —(CH₂)ₙ—(CHOR⁸)ₘ—NH—C(═NR¹³)—NR¹³R¹³, Het-(CH₂)ₘ—(CHOR⁸)ₘ—NH—C(═NR¹³)—NR¹³R¹³, —(CH₂)ₙ—(CHOR⁸)ₘ-Z_{g}-NH—C(═NR¹³)—NR¹³R¹³, Het-(CH₂)ₙ—(CHOR⁸)ₘ-Z_{g}-NH—C(═NR¹³)—NR¹³R¹³, —(CH₂)ₙ-Z_{g}-(CH₂)ₘ—NH—C(═NR¹³)—NR¹³R¹³, Het-(CH₂)ₙ-Z_{g}-(CH₂)ₘNH—C(═NR¹³)—NR¹³R¹³, —(CH₂)ₙ-Z_{g}-(CHOR⁸)ₘ-Z_{g}-NH—C(═NR¹³)—NR¹³R¹³, Het-(CH₂)ₙ-Z_{g}-(CHOR⁸)ₘ-Z_{g}-NH—C(═NR¹³)—NR¹³R¹³, —(CH₂)ₙ—C(═NR¹³)—NR¹³R¹³, Het-(CH₂)ₘ—C(═NH)—NR¹³R¹³, —(CH₂)ₙ-Z_{g}-C(═NH)—NR¹³R¹³, Het-(CH₂)ₘ-Z_{g}-C(═NH)—NR¹³R¹³, —(CH₂)ₙ—NR¹⁰—(CH₂)ₘ(CHOR⁸)ₙ—C(═NR¹³)—NR¹³R¹³, Het-(CH₂)ₘ—NR¹⁰—(CH₂)ₘ(CHOR⁸)ₙ—C(═NR¹³)—NR¹³R¹³, —(CH₂)ₙ—(CHOR⁸)ₘ—C(═NR¹³)—NR¹³R¹³, Het-(CH₂)ₘ—(CHOR⁸)ₘ—C(═NR¹³)—NR¹³R¹³, —(CH₂)ₙ—(CHOR⁸)ₘ-Z_{g}-C(═NR¹³)—NR¹³R¹³, Het-(CH₂)ₙ—(CHOR⁸)ₘ-Z_{g}-C(═NR¹³)—NR¹³R¹³, —(CH₂)ₙ-Z_{g}-(CH₂)ₘ—C(═NHC(═NR¹³)—NR¹³R¹³, Het-(CH₂)ₙ-Z_{g}-(CH₂)ₘ—C(═NR¹³)—NR¹³R¹³, —(CH₂)ₙ-Z_{g}-(CHOR⁸)ₘ-Z_{g}-C(═NR¹³)—NR¹³R¹³, Het-(CH₂)ₙ-Z_{g}-(CHOR⁸)ₘ-Z_{g}-C(═NR¹³)—NR¹³R¹³;
wherein when two —CH₂OR⁸ groups are located 1,2- or 1,3- with respect to each other the R⁸ groups may be joined to form a cyclic mono- or di-substituted 1,3-dioxane or 1,3-dioxolane;
each R⁶ is, independently, —R⁵, —R⁷, —OR⁸, —N(R⁷)₂, —(CH₂)ₘ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷—O—(CH₂)ₘ—CO₂R⁷, —OSO₃H, —O-glucuronide, —O-glucose, 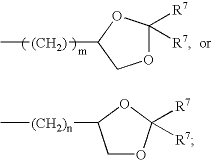
wherein when two R⁶ are —OR¹¹ and are located adjacent to each other on a phenyl ring, the alkyl moieties of the two R⁶ may be bonded together to form a methylenedioxy group, and
wherein when two —CH₂OR⁸ groups are located 1,2- or 1,3- with respect to each other the R⁸ groups may be joined to form a cyclic mono- or di-substituted 1,3-dioxane or 1,3-dioxolane;
each R⁷ is, independently, hydrogen lower alkyl, phenyl, substituted phenyl or —CH₂(CHOR)⁸ₘ—R¹⁰;
each R⁸ is, independently, hydrogen, lower alkyl, —C(═O)—R¹¹, glucuronide, 2-tetrahydropyranyl, or 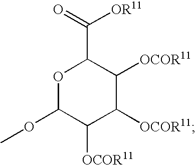
each R⁹ is, independently, —CO₂R⁷, —CON(R⁷)₂, —SO₂CH₃, or —C(═O)R⁷;
each R¹⁰ is, independently, —H, —SO₂CH₃, —CO₂R⁷, —C(═O)NR⁷R⁹, —C(═O)R⁷, or —(CH₂) m—(CHOH)ₙ—CH₂OH;
each Z is, independently, CHOH, C(═O), —(CH₂)ₙ—CHNR⁷R¹⁰, C═NR¹⁰, or NR¹⁰;
each R¹¹ is, independently, lower alkyl;
each R¹² is independently, —SO₂CH₃, —CO₂R⁷, —C(═O)NR⁷R⁹, —C(═O)R⁷, or —CH₂—(CHOH)ₙ—CH₂OH;
each R¹³ is, independently, hydrogen, R⁷, R¹⁰, (CH₂)ₘ—NR⁷R¹⁰, 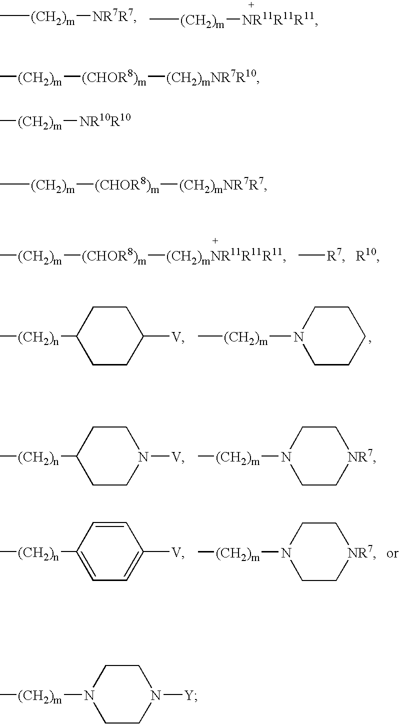
with the proviso that at least one R¹³ must be a group other than hydrogen, R⁷, or R¹⁰;
with the further proviso that NR¹³R¹³ can be joined on itself to form a ring comprising one of the following: 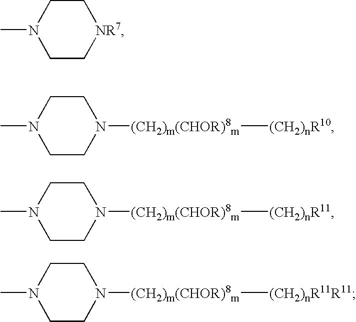
each Het is independently, —NR⁷, —NR¹⁰, —S—, —SO—, or —SO₂—; —O—, —SO₂NH—, —NHSO₂—, —NR⁷CO—, —CONR⁷—;
each g is, independently, an integer from 1 to 6;
each m is, independently, an integer from 1 to 7;
each n is, independently, an integer from 0 to 7;
each Q is, independently, C—R⁵, C—R⁶, or a nitrogen atom, wherein at most three Q in a ring are nitrogen atoms;
each V is, independently, —(CH₂)ₘ—NR⁷R¹⁰, —(CH₂)ₘ—NR⁷R⁷, 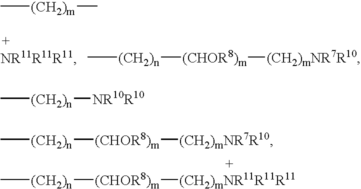
with the proviso that when V is attached directly to a nitrogen atom, then V can also be, independently, R⁷, R¹⁰, or (R¹¹)₂;
wherein for any of the above compounds when two —CH₂OR⁸ groups are located 1,2- or 1,3- with respect to each other the R⁸ groups may be joined to form a cyclic mono- or di-substituted 1,3-dioxane or 1,3-dioxolane.

2. The method of claim 1, wherein each —(CH₂)ₙ-Z_{g}-C(═NH)—NR¹³R¹³ is, independently,
—(CH₂)ₙ—CHNH₂(C═N)—NR¹³R¹³.

3. The method of claim 1, wherein each Het-(CH₂)ₘ—NH—C(═NH)—NR¹³R¹³ is, independently,
—(CH₂)ₙ—NH—C(═NH)NHR¹³.

4. The method of claim 1, wherein each —(CH₂)ₙ-Z_{g}-(CHOR⁸)ₘ-Z_{g}-CONR¹³R¹³ is, independently,
—(CH₂)ₙ—CONHCH₂(CHOH)ₘ—CONHR¹³.

5. The method of claim 1, wherein each Het-(CH₂)ₙ-Z_{g}-(CHOR⁸)ₘ-Z_{g}-CONR¹³R¹³ is, independently,
—NH—C(═O)—CH₂—(CHOH)ₙCH₂CONR¹³R¹³.

6. The method of claim 1, wherein each Het-(CH₂)ₘ-Z_{g}-C(═NH)—NR¹³R¹³ is, independently,
—O—(CH₂)ₘ—NE-C(═NH)—N(R¹³)₂.

7. The method of claim 1, wherein each Het-(CH₂)ₘ-Z_{g}-CONR¹³R¹³ is, independently,
—O—(CH₂)ₘ—CHNH₂—CO₂NR¹³R¹³.

8. The method of claim 1, wherein each R⁵ is, independently,
—O—CH₂CHOHCH₂CONR¹³R¹³
—OCH₂CHOHCH₂CO₂R¹³ OCH₂CH₂CONR¹³R¹³
—OCH₂CH₂NHCOR¹³
—CH₂CH₂CONR¹³R¹³
—OCH₂CH₂CONR¹³R¹³O—(CH₂)ₘ—CO₂R¹³
—(CH₂)ₘ—CO₂R¹³
—OCH₂CH₂CO₂R¹³
—OCH₂CO₂R¹³
—O—(CH₂)ₘ—NH—C(═NH)—NR¹³)₂,
—(CH₂)ₙ—NH—C(═NH)—N(R¹³)₂,
—NHCH₂(CHOH)₂—CCONR¹³R¹³
—OCH₂CO₂R¹³
—NHSO₂(CH₂)₂CONR¹³R¹³
—(CH₂)ₘ—NH—C(═O)—OR¹³
—O—(CH₂)ₘ—NH—C(═O)—OR¹³,
—(CH₂)ₙ—NH—C(═O)—R¹³,
—O—(CH₂)ₘ—NH—C(═O)—R¹³,
—O—CH₂C(═O)NR¹³R¹³
—CH₂NCO₂R¹³
—NHCO₂R¹³
—OCH₂CH₂CH₂CH₂CONR¹³R¹³
—SO₂CH₂CH₂CONR¹³R¹³
—OCH₂CH₂CHOHCH₂CONR¹³R¹³
—OCH₂CH₂NHCO₂R¹³
—NH—C(═NH₂)—NR¹³R¹³
—OCH₂-(α-CHOH)₂—CONR¹³R¹³
—OCH₂CHOHCH₂CONHR¹³
—(CH₂)ₘ—CHOH—CH₂—NHCO₂R¹³
—O—(CH₂)ₘ—CHOH—CH₂—CO₂R¹³
—(CH₂)ₘ—NHC(O)OR¹³
—O—(CH₂)ₘ—NHC(O)OR¹³
—OCH₂CH₂CH₂CONHR¹³
—OCH₂CH₂NHCH₂(CHOH)₂CH₂CONHR¹³
—OCH₂CH₂CONH(CH₂[(CHOH)₂CH₂NH₂)]₂,
—(CH₂)₄—NHCO₂R¹³,
—(CH₂)₄—CONR¹³R¹³,
—(CH₂)₄—CO₂R¹³
—OCH₂CH₂CONHSOCH₂CH₂N(CH₃)₂
—O—(CH₂)ₘ—C(═NH)—N(R¹³)₂,
—(CH₂)ₙ—C(═NH)—N(R¹³)₂,
—(CH₂)₃—NHCO₂R¹³, —(CH₂)₃CONHCO₂R¹³
—O—(CH₂)ₘ—NH—NH—C(═NH)—N(R¹³)₂,
—(CH₂)ₙ—NH—NH—C(═NH)—N(R¹³)₂, or
—O—CH₂—CHOH—CH₂—NH—C(═NH)—N(R¹³)₂.

9. The prophylactic treatment method of claim 1, wherein the pathogen is Bacillus anthracis.

10. The prophylactic treatment method of claim 1, wherein the pathogen is Variola major.

11. The prophylactic treatment method of claim 1, wherein the pathogen is Yersinia pestis.

12. The prophylactic treatment method of claim 1, wherein the pathogen is Francisella tularensis.

13. The prophylactic treatment method of claim 1, wherein the pathogen is a gram negative bacteria.

14. The prophylactic treatment method of claim 13, wherein the gram negative bacteria is selected from the group consisting of Brucella species, Burkholderia pseudomallei, Burkholderia mallei, Coxiella burnetii and Rickettsia.

15. The prophylactic treatment method of claim 1 wherein the pathogen is an alphavirus, a flavivirus or a bunyavirus.

16. The prophylactic treatment method of claim 1, wherein the pathogen is ricin toxin from Ricinus communis, epsilon toxin of Clostridium perfringens or Staphylococcal enterotoxin B.

17. The prophylactic treatment method of claim 1, wherein the pathogen is Mycobacterium tuberculosis bacteria.

18. The prophylactic treatment method of claim 1, wherein the pathogen is an influenza virus, rhinovirus, adenovirus or respiratory syncytial virus.

19. The prophylactic treatment method of claim 1, wherein the pathogen is coronavirus.

20. The prophylactic treatment method of claim 1, wherein the sodium channel blocker or pharmaceutically acceptable salt thereof is administered in an aerosol suspension of respirable particles which the individual inhales.

21. The prophylactic treatment method of claim 1, wherein the sodium channel blocker or a pharmaceutically acceptable salt is administered post-exposure to the one or more airborne pathogens.

22. A prophylactic treatment method for reducing the risk of infection from an airborne pathogen which can cause a disease in a human, said method comprising administering an effective amount of a sodium channel blocker according to Formula (I) or a pharmaceutically acceptable salt thereof to the lungs of the human who may be at risk of infection from the airborne pathogen but is asymptomatic for the disease, wherein the effective amount of sodium channel blocker or a pharmaceutically acceptable salt is sufficient to reduce the risk of infection in the human, wherein Formula (I) is 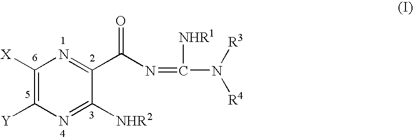
where X is hydrogen, halogen, trifluoromethyl, lower alkyl, unsubstituted or substituted phenyl, lower alkyl-thio, phenyl-lower alkyl-thio, lower alkyl-sulfonyl, or phenyl-lower alkyl-sulfonyl; Y is hydrogen, hydroxyl, mercapto, lower alkoxy, lower alkyl-thio, halogen, lower alkyl, unsubstituted or substituted mononuclear aryl, or —N(R²)₂; R¹ is hydrogen or lower alkyl; each R² is, independently, —R⁷, —(CH₂)ₘ—OR⁸, —(CH₂)ₘ—NR⁷R¹⁰, —(CH₂)ₙ(CHOR⁸)(CHOR⁸)ₙ—CH₂OR⁸, —(CH₂CH₂O)ₘ—R⁸, —(CH₂CH₂O)ₘ—CH₂CH₂NR⁷R¹⁰, —(CH₂)ₙ—C(═O)NR⁷R¹⁰, —(CH₂)ₙ-Z_{g}-R⁷, —(CH₂)ₘ—NR¹⁰—CH₂(CHOR⁸)(CHOR⁸), —CH₂OR⁸, —(CH₂)ₙ—CO₂R⁷, or 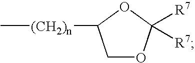
wherein when two —CH₂OR⁸ groups are located 1,2- or 1,3- with respect to each other the R⁸ groups may be joined to form a cyclic mono- or di-substituted 1,3-dioxane or 1,3-dioxolane;
R³ and R⁴ are each, independently, hydrogen, a group represented by formula (A), lower alkyl, hydroxy lower alkyl, phenyl, phenyl-lower alkyl, (halophenyl)-lower alkyl, lower-(alkylphenylalkyl), lower (alkoxyphenyl)-lower alkyl, naphthyl-lower alkyl, or pyridyl-lower alkyl, with the proviso that at least one of R³ and R⁴ is a group represented by formula (A): 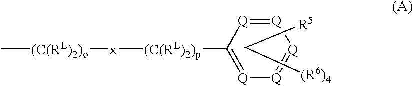
wherein
each R^{L} is, independently, —R⁷, —(CH₂)ₙ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose, 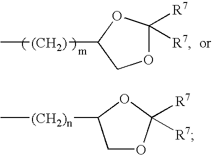
wherein when two —CH₂OR⁸ groups are located 1,2- or 1,3- with respect to each other the R⁸ groups may be joined to form a cyclic mono- or di-substituted 1,3-dioxane or 1,3-dioxolane;
each o is, independently, an integer from 0 to 10;
each p is an integer from 0 to 10;
with the proviso that the sum of o and p in each contiguous chain is from 1 to 10;
each x is, independently, O, NR¹⁰, C(═O), CHOH, C(═N—R¹⁰), CHNR⁷R¹⁰, or represents a single bond;
each R⁵ is independently, —(CH₂)ₙ—CO₂R¹³, Het-(CH₂)ₘ—CO₂R¹³, —(CH₂)ₙ-Z_{g}-CO₂R¹³, Het-(CH₂)ₘ-Z_{g}-CO₂R¹³, —(CH₂)ₙ—NR¹⁰—(CH₂)ₘ(CHOR⁸)ₙ—CO₂R¹³, Het-(CH₂)ₘ—NR¹⁰—(CH₂)ₘ(CHOR⁸)ₙ—CO₂R¹³, —(CH₂)ₙ—(CHOR⁸)ₘ—CO₂R¹³, Het-(CH₂)ₘ—(CHOR⁸)ₘ—CO₂R¹³, —(CH₂)ₙ—(CHOR⁸)ₘ-Z_{g}-CO₂R¹³, Het-(CH₂)ₙ—(CHOR⁸)ₘ-Z_{g}-CO₂R¹³, —(CH₂)ₙ-Z_{g}-(CH₂)ₘ—CO₂R¹³, —(CH₂)ₙ-Z_{g}-(CH₂)ₘ—CO₂R¹³, —(CH₂)ₙ-Z_{g}(CHOR⁸)ₘ-Z_{g}-CO₂R¹³, Het-(CH₂)ₙ-Z_{g}-(CHOR⁸)ₘ-Z_{g}-CO₂R¹³, —(CH₂)ₙ—CONH—C(═NR¹³)—NR¹³R¹³, Het-(CH₂)ₙ—CO—NH—C(═NR¹³)—NR¹³R¹³, —(CH₂)ₙ-Z_{g}-CONH—C(═NR¹³)—NR¹³R¹³, Het-(CH₂)ₙ-Z_{g}-CONH—C(═NR¹³)—NR¹³R¹³, —(CH₂)ₙ—NR¹⁰—(CH₂)ₘ(CHOR⁸)ₙ—CONH—C(═NR¹³)—NR¹³R¹³, Het-(CH₂)ₙ—NR¹⁰—(CH₂)ₘ(CHOR⁸)ₙ—CONH—C(═NR¹³)—NR¹³R¹³, —(CH₂)ₙ—(CHOR⁸)ₘ—CONH—C(═NR¹³)—NR¹³R¹³, Het-(CH₂)ₙ—(CHOR⁸)ₘ—CONH—C(═NR¹³)—NR¹³R¹³, —(CH₂)ₙ—(CHOR⁸)ₘ-Z_{g}-CONH—C(═NR¹³)—NR¹³R¹³, Het-(CH₂)ₙ—(CHOR⁸)ₘ-Z_{g}-CONH—C(═NR¹³)—NR¹³R¹³, —(CH₂)ₙ-Z_{g}-(CH₂)ₘCONH—C(═NR¹³)—NR¹³R¹³, Het-(CH₂)ₙ-Z_{g}-(CH₂)ₘCONH—C(═NR¹³)—NR¹³R¹³, —(CH₂)ₙ-Z_{g}-(CHOR⁸)ₘ-Z_{g}-CONH—C(═NR¹³)—NR¹³R¹³, Het-(CH₂)ₙ-Z_{g}-(CHOR⁸)ₘ-Z_{g}-CONH—C(═NR¹³)—NR¹³R¹³, —(CH₂)ₙ—CONR⁷—CONR¹³R¹³, Het-(CH₂)ₙ—CONR⁷—CONR¹³R¹³, —(CH₂)ₙ-Z_{g}-CONR⁷—CONR¹³R¹³, —(CH₂)ₙ-Z_{g}-CONR⁷—CONR¹³R¹³, —(CH₂)ₙ—NR¹⁰—(CH₂)ₘ(CHOR⁸)ₙ—CONR⁷—CONR¹³R¹³, Het-(CH₂)ₙ—NR¹⁰—(CH₂)ₘ(CHOR⁸)ₙ—CONR⁷—CONR¹³R¹³, —(CH₂)ₙ—(CHOR⁸)ₘ, —CONR⁷—CONR¹³R¹³, Het-(CH₂)ₙ—(CHOR⁸)ₘ—CONR⁷—CONR¹³R¹³, —(CH₂)ₙ—(CHOR⁸)ₘ-Z_{g}-CONR⁷—CONR¹³R¹³, Het-(CH₂)ₙ—(CHOR⁸)ₘ-Z_{g}-CNR⁷—CONR¹³R¹³, —(CH₂)ₙ-Z_{g}-(CH₂)ₘCONR⁷—CONR¹³R¹³, Het-(CH₂)ₙ-Z_{g}-(CH₂)ₘCONR⁷—CONR¹³R¹³, —(CH₂)ₙ-Z_{g}(CHOR⁸)ₘ-Z_{g}-CONR⁷—CONR¹³R¹³, Het-(CH₂)ₙ-Z_{g}(CHOR⁸)ₘ-Z_{g}-CONR⁷—CONR¹³R¹³, —(CH₂)ₙ—CONR⁷ SO₂NR¹³R¹³, Het-(CH₂)ₘ—CONR⁷SO₂NR¹³R¹³, —(CH₂)ₙ-Z_{g}-CONR⁷SO₂NR¹³R¹³, Het-(CH₂)ₘ-Z_{g}-CONR⁷SO₂NR¹³R¹³, —(CH₂)ₙ—NR¹⁰—(CH₂)ₘ(CHOR⁸)ₙ—CONR⁷SO₂NR¹³R¹³, Het-(CH₂)ₘ—NR¹⁰—(CH₂)ₘ(CHOR⁸)ₙ—CONR⁷SO₂NR¹³R¹³, —(CH₂)ₙ—(CHOR⁸)ₘ—CONR⁷SO₂NR¹³R¹³, Het-(CH₂)ₘ—(CHOR⁸)ₘ—CONR⁷SO₂NR¹³R¹³, —(CH₂)ₙ—(CHOR⁸)ₘ-Z_{g}-CONR⁷SO₂NR¹³R¹³, Het-(CH₂)ₙ—(CHOR⁸)ₘ-Z_{g}-CONR⁷SO₂NR¹³R¹³, —(CH₂)ₙ-Z_{g}-(CH₂)ₘCONR⁷SO₂NR¹³R¹³, Het-(CH₂)ₙ-Z_{g}-(CH₂)ₘCONR⁷SO₂NR¹³R¹³, —(CH₂)ₙ-Z_{g}-(CHOR⁸)ₘ-Z_{g}-CONR⁷SO₂NR¹³R¹³, Het-(CH₂)ₙ-Z_{g}-(CHOR⁸)ₘ-Z_{g}-CONR⁷SO₂NR¹³R¹³, —(CH₂)ₙ—SO₂NR¹³R¹³, Het-(CH₂)ₘ—SO₂NR¹³R¹³, —(CH₂)ₙ-Z_{g}-SO₂NR¹³R¹³, Het-(CH₂)ₘ-Z_{g}-SO₂NR¹³R¹³, —(CH₂)ₙ—NR¹⁰—(CH₂)ₘ(CHOR⁸)ₙ—SO₂NR¹³R¹³, Het-(CH₂)ₘ—NR¹⁰—(CH₂)ₘ(CHOR⁸)ₙ—SO₂NR¹³R¹³, —(CH₂)ₙ—(CHOR⁸)ₘ—SO₂NR¹³R¹³, Het-(CH₂)ₘ—(CHOR⁸)ₘ—SO₂NR¹³R¹³, —(CH₂)ₙ—(CHOR⁸)ₘ-Z_{g}-SO₂NR¹³R¹³, Het-(CH₂)ₙ—(CHOR⁸)ₘ-Z_{g}-SO₂NR¹³R¹³, —(CH₂)ₙ-Z_{g}-(CH₂)ₘ—SO₂NR¹³R¹³, Het-(CH₂)ₙ-Z_{g}-(CH₂)ₘ—SO₂NR¹³R¹³, —(CH₂)ₙ-Z_{g}-(CHOR⁸)ₘ-Z_{g}-SO₂NR¹³R¹³, Het-(CH₂)ₙ-Z_{g}-(CHOR⁸)ₘ-Z_{g}-SO₂NR¹³R¹³, —(CH₂)ₙ—CONR¹³R¹³, Het-(CH₂)ₘ—CONR¹³R¹³, —(CH₂)ₙ-Z_{g}-CONR¹³R¹³, Het-(CH₂)ₘ-Z_{g}-CONR¹³R¹³, —(CH₂)ₙ—NR¹⁰—(CH₂)ₘ(CHOR⁸)ₙ—CONR¹³R¹³m Het-(CH₂)ₘ—NR¹⁰—(CH₂)ₘ(CHOR⁸)ₙ—CONR¹³R¹³, (CHOR⁸)ₘ—CONR¹³R¹³, Het-(CH₂)ₘ—(CHOR⁸)ₘ—CONR¹³R¹³, —(CH₂)ₙ—(CHOR⁸)ₘ-Z_{g}-CONR¹³R¹³, Het-(CH₂)ₙ—(CHOR⁸)ₘ-Z_{g}-CONR¹³R¹³, —(CH₂)ₙ-Z_{g}-(CH₂)ₘCONR¹³R¹³, Het-(CH₂)ₘ-Z_{g}-(CH₂)ₘCONR¹³R¹³, —(CH₂)ₙ-Z_{g}-(CHOR⁸)ₘ-Z_{g}-CONR¹³R¹³, Het-(CH₂)ₙ-Z_{g}-(CHOR⁸)ₘ-Z_{g}-CONR¹³R¹³, —(CH₂)ₙ—CONR⁷COR¹³, Het-(CH₂)ₘ—CONR⁷COR¹³, —(CH₂)ₙ-Z_{g}-CONR⁷COR¹³, Het-(CH₂)ₘ-Z_{g}-CONR⁷COR¹³, —(CH₂)ₙ—NR¹⁰—(CH₂)ₘ(CHOR⁸)ₙ—CONR⁷COR¹³, Het-(CH₂)ₘ—NR¹⁰—(CH₂)ₘ(CHOR⁸)ₙ—CONR⁷COR¹³, —(CH₂)ₙ—(CHOR⁸)ₘ—CONR⁷COR¹³, Het-(CH₂)ₘ—(CHOR⁸)ₘ—CONR⁷COR¹³, —(CH₂)ₙ—(CHOR⁸)ₘ-Z_{g}-CONR⁷COR¹³, Het-(CH₂)ₙ—(CHOR⁸)ₘ-Z_{g}-CONR⁷COR¹³, —(CH₂)ₙ-Z_{g}-(CH₂)ₘCONR⁷COR¹³, —(CH₂)ₙ-Z_{g}-(CH₂)ₘCONR⁷COR¹³, Het-(CH₂)ₙ-Z_{g}-(CHOR⁸)ₘ-Z_{g}-CONR⁷COR¹³, —(CH₂), —CONR⁷CO₂R¹³, —(CH₂)ₙ-Z_{g}-CONR⁷CO₂R¹³, Het-(CH₂)ₘ-Z_{g}-CONR⁷CO₂R¹³, —(CH₂)ₙ—NR¹⁰—(CH₂)ₘ(CHOR⁸)ₙ—CONR⁷CO₂R¹³, Het-(CH₂)ₘ—NR¹⁰—(CH₂)ₘ(CHOR⁸)ₙ—CONR⁷CO₂R¹³, —(CH₂)ₙ—(CHOR⁸)ₘ—CONR⁷CO₂R¹³, Het-(CH₂)ₘ—(CHOR⁸)ₘ—CONR⁷CO₂R¹³, —(CH₂)ₙ—(CHOR⁸)ₘ-Z_{g}-CONR⁷CO₂R¹³, Het-(CH₂)ₙ—(CHOR⁸)ₘ-Z_{g}-CONR⁷CO₂R¹³, —(CH₂)ₙ-Z_{g}-(CH₂)ₘCONR⁷CO₂R¹³, Het-(CH₂)ₙ-Z_{g}-(CH₂)ₘCONR⁷CO₂R¹³, —(CH₂)ₙ-Z_{g}-(CHOR⁸)ₘ-Z_{g}-CONR⁷CO₂R¹³, Het-(CH₂)ₙ-Z_{g}-(CHOR⁸)ₘ-Z_{g}-CONR⁷CO₂R¹³, —(CH₂)ₙ—NH—C(═NR¹³)—NR¹³R¹³, Het-(CH₂)ₘ—NH—C(═NR¹³)—NR¹³R¹³, —(CH₂)ₙ-Z_{g}-NH—C(═NR¹³)—NR¹³R¹³, Het-(CH₂)ₘ-Z_{g}-NH—C(═NR¹³)—NR¹³R¹³, —(CH₂)ₙ—NR¹⁰—(CH₂)ₘ(CHOR⁸)ₙ—NH—C(═NR¹³)—NR¹³R¹³, Het-(CH₂)ₘ—NR¹⁰—(CH₂)ₘ(CHOR⁸)ₙ—NH—C(═NR¹³)—NR¹³R¹³, —(CH₂)ₙ—(CHOR⁸)ₘ—NH—C(═NR¹³)—NR¹³R¹³, Het-(CH₂)ₘ—(CHOR⁸)ₘ—NH—C(═NR¹³)—NR¹³R¹³, —(CH₂)ₙ—(CHOR⁸)ₘ-Z_{g}-NH—C(═NR¹³)—NR¹³R¹³, Het-(CH₂)ₙ—(CHOR⁸)ₘ-Z_{g}-NH—C(═NR¹³)—NR¹³R¹³, —(CH₂)ₙ-Z_{g}-(CH₂)ₘ—NH—C(═NR¹³)—NR¹³R¹³, Het-(CH₂)ₙ-Z_{g}-(CH₂)ₘ—NH—C(═NR¹³)—NR¹³R¹³, —(CH₂)ₙ-Z_{g}-(CHOR⁸)ₘ-Z_{g}-NH—C(═NR¹³)—NR¹³R¹³, Het-(CH₂)ₙ-Z_{g}-(CHOR⁸)ₘ-Z_{g}-NH—C(═NR¹³)—NR¹³R¹³, —(CH₂)ₙ—C(—NR¹³)—NR¹³R¹³, Het-(CH₂)ₘ—C(═NH)—NR¹³R¹³, —(CH₂)ₙ-Z_{g}-C(═NH)—NR¹³R¹³, Het-(CH₂)ₘ-Z_{g}-C(═NH)—NR¹³R¹³, —(CH₂)ₙ—NR¹⁰—(CH₂)ᵣ(CHOR⁸)ₙ—C(═NR¹³)—NR¹³R¹³, Het-(CH₂)ₘ—NR¹⁰—(CH₂)ₘ(CHOR⁸)ₙ—C(═NR¹³)—NR¹³R¹³, —(CH₂)ₙ—(CHOR⁸)ₘ—C(═NR¹³)—NR¹³R¹³, Het-(CH₂)ₘ—(CHOR⁸)ₘ—C(═NR¹³)—NR¹³R¹³, —(CH₂)ₙ—(CHOR⁸)ₘ-Z_{g}-C(═NR¹³)—NR¹³R¹³, Het-(CH₂)ₙ—(CHOR⁸)ₘ-Z_{g} C(═NR¹³)—NR¹³R¹³, —(CH₂)ₙ-Z_{g}-(CH₂)ₘ—C(═NHC(═NR¹³)—NR¹³R¹³, Het-(CH₂)ₙ-Z_{g}-(CH₂)ₘ—C(═NR¹³)—NR¹³R¹³, —(CH₂)ₙ-Z_{g}-(CHOR⁸)ₘ-Z_{g}-C(═NR¹³)—NR¹³R¹³, Het-(CH₂)ₙ-Z_{g}-(CHOR⁸)ₘ-Z_{g}-C(═NR¹³)—NR¹³R¹³;
wherein when two —(CH₂OR⁸ groups are located 1,2- or 1,3- with respect to each other the R⁸ groups may be joined to form a cyclic mono- or di-substituted 1,3-dioxane or 1,3-dioxolane;
each R⁶ is, independently, —R⁵, —R⁷, —OR⁸, —N(R⁷)₂, —(CH₂)ₘ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸), —CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose, 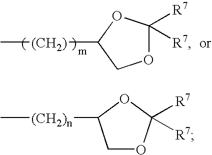
wherein when two, R⁶ are —OR¹¹ and are located adjacent to each other on a phenyl ring, the alkyl moieties of the two R⁶ may be bonded together to form a methylenedioxy group, and
wherein when two —CH₂OR⁸ groups are located 1,2- or 1,3- with respect to each other the R⁸ groups may be joined to form a cyclic mono- or di-substituted 1,3-dioxane or 1,3-dioxolane;
each R⁷ is, independently, hydrogen lower alkyl, phenyl, substituted phenyl or —CH₂(CHOR)⁸ₘ—R¹⁰;
each R⁸ is, independently, hydrogen, lower alkyl, —C(═O)—R¹¹, glucuronide, 2-tetrahydropyranyl, or 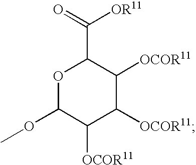
each R⁹ is, independently, —CO₂R⁷, —CON(R⁷)₂, —SO₂CH₃, or —C(═O)R⁷;
each R¹⁰ is, independently, —H, —SO₂CH₃, —CO₂R⁷, —C(═O)NR⁷R⁹, —C(═O)R⁷, or —(CH₂) m—(CHOH)ₙ—CH₂OH;
each Z is, independently, CHOH, C(═O), —(CH₂)ₙ—, CHNR⁷R¹⁰, C═NR¹⁰, or NR¹⁰;
each R¹¹ is, independently, lower alkyl;
each R¹² is independently, —SO₂CH₃, —CO₂R⁷, —C(═O)NR⁷R⁹, —C(═O)R⁷, or —CH₂—(CHOH), —CH₂OH;
each R¹³ is, independently, hydrogen, R⁷, R¹⁰, —(CH₂)ₘ—NR⁷R¹⁰, —(CH₂)ₘ—NR⁷R¹⁰, 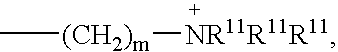
—(CH₂)ₘ—(CHOR⁸)ₘ—(CH₂)ₘNR⁷R¹⁰, —(CH₂)ₘ—NR¹⁰R¹⁰
—(CH₂)ₘ—(CHOR⁸)ₘ—(CH₂)ₘNR⁷R⁷, 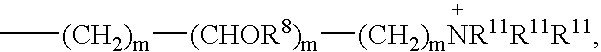
—R⁷, R¹⁰, 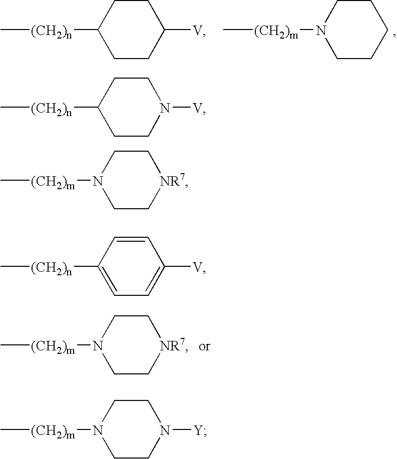
with the proviso that at least one R¹³ must be a group other than hydrogen, R⁷, or
R¹⁰;
with the further proviso that NR¹³R¹³ can be joined on itself to form a ring comprising one of the following: 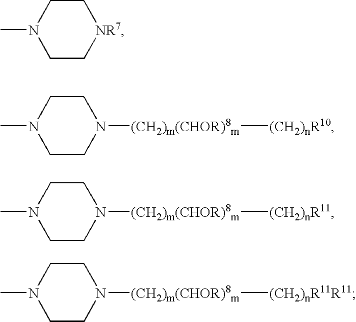
each Het is independently, —NR⁷, —NR¹⁰, —S—, —SO—, or —SO₂—; —O—, —SO₂NH—, —NHSO₂—, —NR⁷CO—, —CONR⁷—;
each g is, independently, an integer from 1 to 6;
each m is, independently, an integer from 1 to 7;
each n is, independently, an integer from 0 to 7;
each Q is, independently, C—R⁵, C—R⁶, or a nitrogen atom, wherein at most three Q in a ring are nitrogen atoms;
each V is, independently, —(CH₂)ₘ—NR⁷R¹⁰, —(CH₂)ₘ—NR⁷R⁷, —(CH₂)ₘ— 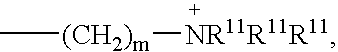
+NR¹¹R¹¹R¹¹, —(CH₂)ₙ—(CHOR⁸)ₘ—(CH₂)ₘNR⁷R¹⁰, —(CH₂)ₙ—NR¹⁰R¹⁰ 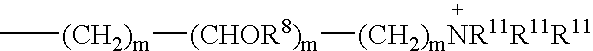
—(CH₂)ₙ—(CHOR⁸)ₘ—(CH₂)ₘNR⁷R⁷, —(CH₂)ₙ—(CHOR⁸)ₘ—(CH₂)ₘNR¹¹R¹¹R¹¹
with the proviso that when V is attached directly to a nitrogen atom, then V can also be, independently, R⁷, R¹⁰, or (R¹¹)₂;
wherein for any of the above compounds when two —CH₂OR⁸ groups are located 1,2- or 1,3- with respect to each other the R⁸ groups may be joined to form a cyclic mono- or di-substituted 1,3-dioxane or 1,3-dioxolane.

23. The method of claim 22, wherein each —(CH₂)ₙ-Z_{g}-C(═NH)—NR¹³R¹³ is, independently,
—(CH₂)ₙ—CHNH₂(C═N)—NR¹³R¹³.

24. The method of claim 22, wherein each Het-(CH₂)ₘ—NH—C(═NH)—NR¹³R¹³ is, independently,
—(CH₂)ₙ—NH—C(═NH)NHR¹³.

25. The method of claim 22, wherein each —(CH₂)ₙ-Z_{g}-(CHOR⁸)ₘ-Z_{g}-CONR¹³R¹³ is, independently,
—(CH₂)ₙ—CONHCH₂(CHOH)ₘ—CONHR¹³.

26. The method of claim 22, wherein each Het-(CH₂)ₙ-Z_{g}-(CHOR⁸)ₘ-Z_{g}-CONR¹³R¹³ is, independently,
—NH—C(═O)—CH₂—(CHOH)ₙCH₂CONR¹³R¹³.

27. The method of claim 22, wherein each Het-(CH₂)ₘ-Z_{g}-C(═NH)—NR¹³R¹³ is, independently,
—O—(CH₂)ₘ—NH—C(═NH)—N(R¹³)₂.

28. The method of claim 22, wherein each Het-(CH₂)ₘ-Z_{g}-CONR¹³R¹³ is, independently,
—O—(CH₂)ₘ—CHNH₂—CO₂NR¹³R¹³.

29. The method of claim 22, wherein each R⁵ is, independently,
—O—CH₂CHOHCH₂CONR¹³R¹³
—OCH₂CHOHCH₂CO₂R¹³ OCH₂CH₂CONR¹³R¹³
—OCH₂CH₂NHCOR¹³
—CH₂CH₂CONR¹³R¹³
—OCH₂CH₂CONR¹³R¹³O—(CH₂)ₘ—CO₂R¹³
—(CH₂)ₘ—CO₂R¹³
—OCH₂CH₂CO₂R¹³
—OCH₂CO₂R¹³
—O—(CH₂)ₘ—NH—C(═NH)—NR¹³)₂,
—(CH₂)ₙ—NH—C(═NH)—N(R¹)₂,
—NHCH₂(CHOH)₂—CCONR¹³R¹³
—OCH₂CO₂R¹³
—NHSO₂(CH₂)₂CONR¹³R¹³
—(CH₂)ₘ—NH—C(═O)—OR¹³
—O—(CH₂)ₘ—NH—C(═O)—OR¹³,
—(CH₂)ₙ—NH—C(═O)—R¹³;
—O—(CH₂)ₘ—NH—C(═O)—R¹³,
—O—CH₂C(═O)NR¹³R¹³,
—CH₂NCO₂R¹³,
—NHCO₂R¹³,
—OCH₂CH₂CH₂CH₂CONR¹³R¹³
—SO₂CH₂CH₂CONR¹³R¹³
—OCH₂CH₂CHOHCH₂CONR¹³R¹³
—OCH₂CH₂NHCO₂R¹³
—NH—C(═NH₂)—NR¹³R¹³
—OCH₂-(α-CHOH)₂—CONR¹³R¹³
—OCH₂CHOHCH₂CONHR¹³
—(CH₂)ₘ—CHOH—CH₂—NHCO₂R¹³
—O—(CH₂)ₘ—CHOH—CH₂—CO₂R¹³
—(CH₂)ₘ—NHC(O)OR¹³
—O—(CH₂)ₘ—NHC(O)OR¹³
—OCH₂CH₂CH₂CONHR¹³
—OCH₂CH₂NHCH₂(CHOH)₂CH₂CONHR¹³
—OCH₂CH₂CONH(CH₂[(CHOH)₂CH₂NH₂)]₂,
—(CH₂)₄—NHCO₂R¹³,
—(CH₂)₄—CONR¹³R¹³,
—(CH₂)₄—CO₂R¹³
—OCH₂CH₂CONHSOCH₂CH₂N(CH₃)₂
—O—(CH₂)ₘ—C(═NH)—N(R¹³)₂,
—(CH₂)ₙ—C(═NH)—N(R¹³)₂,
—(CH₂)₃—NHCO₂R¹³—(CH₂)₃CONHCO₂R¹³
—O—(CH₂)ₘ—NH—NH—C(═NH)—N(R¹³)₂,
—(CH₂)ᵣ—NH—NH—C(═NH)—N(R¹³)₂, or
—O—CH₂—CHOH—CH₂—NH—C(═NH)—N(R¹³)₂.

30. The prophylactic treatment method of claim 22, wherein the airborne pathogen is Bacillus anthracis and the disease is anthrax.

31. The prophylactic treatment method of claim 22, wherein the airborne pathogen is Variola major and the disease is small pox.

32. The prophylactic treatment method of claim 22, wherein the airborne pathogen is Yersinia pestis and the disease is plague.

33. The prophylactic treatment method of claim 22, wherein the airborne pathogen is a gram negative bacteria.

34. The prophylactic treatment method of claim 33, wherein the gram negative bacteria is selected from the group consisting of Brucella species, Burkholderia pseudomallei, Burkholderia mallei, and Coxiella burnetii.

35. The prophylactic treatment method of claim 22, wherein the airborne pathogen is an alphavirus, a flavivirus or a bunyavirus.

36. The prophylactic treatment method of claim 22, wherein the airborne pathogen is ricin toxin from Ricinus communis, epsilon toxin of Clostridium perfringens or Staphylococcal enterotoxin B.

37. The prophylactic treatment method of claim 22, wherein the airborne pathogen is Mycobacterium tuberculosis bacteria.

38. The prophylactic treatment method of claim 22, wherein the airborne pathogen is an influenza virus, rhinovirus, adenovirus or respiratory syncytial virus.

39. The prophylactic treatment of claim 22, wherein the airborne pathogen is coronavirus and the disease is severe acute respiratory syndrome.

40. The prophylactic treatment method of claim 22, wherein the sodium channel blocker or pharmaceutically acceptable salt thereof is administered in an aerosol suspension of respirable particles which the human inhales.

41. The prophylactic treatment method of claim 22, wherein the sodium channel blocker or a pharmaceutically acceptable salt is administered post-exposure to the airborne pathogen.
